SEQUENCE LISTING (54 sequences)

SEQ ID NO: 1 - protein, 140 aa, synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 140 aa, synthetic construct
MDVFMKGLSKAKEGVVAAAEKTKQGVAEAAGKTKEGVLYVGSKTKEGVVHGVTTVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 3 - protein, 140 aa, synthetic construct
MDVFMKGLSKAKEGVVAAAEKTKQGVAEAAGKTKEGVLYVGSKTKEGVVHGVTTVAEKTKEQVTNVGGAVVTGVTAVAQKTVEGAGNIAAATGFVKKDHLGKSEEGAPQEGILEDMPVDPDNEAYEMPSEEGYQDYEPEA

SEQ ID NO: 4 - protein, 140 aa, synthetic construct [sequence omitted]

SEQ ID NO: 5 - protein, 441 aa, synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 412 aa, synthetic construct
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 410 aa, synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 8 - protein, 381 aa, synthetic construct
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 9 - protein, 383 aa, synthetic construct
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 10 - protein, 352 aa, synthetic construct
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 11 - protein, 430 aa, synthetic construct
MADPRQEFDTMEDHAGDYTLLQDQEGDMDHGLKESPPQPPADDGAEEPGSETSDAKSTPTAEDVTAPLVDERAPDKQAAAQPHTEIPEGITAEEAGIGDTPNQEDQAAGHVTQARVASKDRTGNDEKKAKGADGKTGAKIATPRGAASPAQKGTSNATRIPAKTTPSPKTPPGSGEPPKSGERSGYSSPGSPGTPGSRSRTPSLPTPPTREPKKVAVVRTPPKSPSASKSRLQTAPVPMPDLKNVRSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 12 - protein, 414 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 13 - protein, 414 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSPDEPLRSRKVFVGRCTEDMTAEELQQFFCQYGEVVDVFIPKPFRAFAFVTFADDKVAQSLCGEDLIIKGISVHISNAEPKHNSNRQLERSGRFGGNPGGFGNQGGFGNSRGGGAGLGNNQGGNMGGGMNFGAFSINPAMMAAAQAALQSSWGMMGMLASQQNQSGPSGNNQSQGSMQREPNQAFGSGNNSYSGSNSGAPLGWGSASNAGSGSGFNGGFGSSMDSKSSGWGM

SEQ ID NO: 14 - protein, 798 aa, synthetic construct
MIDYTAAGFTLLQGAHLYAPEDRGICDVLVANGKIIAVASNIPSDIVPNCTVVDLSGQILCPGFIDQHVHLIGGGGEAGPTTRTPEVALSRLTEAGVTSVVGLLGTDSISRHPESLLAKTRALNEEGISAWMLTGAYHVPSRTITGSVEKDVAIIDRVIGVKCAISDHRSAAPDVYHLANMAAESRVGGLLGGKPGVTVFHMGDSKKALQPIYDLLENCDVPISKLLPTHVNRNVPLFYQALEFARKGGTIDITSSIDEPVAPAEGIARAVQAGIPLARVTLSSDGNGSQPFFDDEGNLTHIGVAGFETLLETVQVLVKDYDFSISDALRPLTSSVAGFLNLTGKGEILPGNDADLLVMTPELRIEQVYARGKLMVKDGKACVKGTFETAGGSGGTGGSGGTGGSGGTGGSGGTGGKPIPNPLLGLDSTGSGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 15 - protein, 201 aa, synthetic construct
MSSEKEELRERLVKICVELAKLKGDDTLKAAEAAEEAFRLVVLAAMLAGIDSSEVLELAIRLIKTCVVLAAMEGYDISEACRAAAEAFTRVAMAALRAGITSSLVLKAAIELIKECVLNAAVEGYDISEACRAAAEAFKRVAEAAKRAGITSLETLLRAIEEIRKRVEEAQREGNDISEACRQAAEEFRKKAEELKRRGDV

SEQ ID NO: 16 - protein, 170 aa, synthetic construct
TKEERVLLMKVAILAIVAAKKGNTDEVRKALELALLIAKVSGTTEAVKLALEVVARVAIEAARRGNTDAVREALEVALEIARESGTTEAVKLALEVVARVAIEAARRGNTEAVVEALLVALEIAKESGTEEAVRLALEVVKRVSNEALKQGNVDAVKVALEVRKMIEELS

SEQ ID NO: 17 - protein, 760 aa, synthetic construct
MKVLIVESEFLHQDTWVGNAVERLADALSQQNVTVIKSTSFDDGFAILSSNEAIDCLMFSYQMEHPDEHQNVRQLIGKLHERQQNVPVFLLGDREKALAAMDRDLLELVDEFAWILEDTADFIAGRAVAAMTRYRQQLLPPLFSALMKYSDIHEYSWAAPGHQGGVGFTKTPAGRFYHDYYGENLFRTDMGIERTSLGSLLDHTGAFGESEKYAARVFGADRSWSVVVGTSGSNRTIMQACMTDNDVVVVDRNCHKSIEQGLMLTGAKPVYMVPSRNRYGIIGPIYPQEMQPETLQKKISESPLTKDKAGQKPSYCVVTNCTYDGVCYNAKEAQDLLEKTSDRLHFDEAWYGYARFNPIYADHYAMRGEPGDHNGPTVFATHSTHKLLNALSQASYIHVREGRGAINFSRFNQAYMMHATTSPLYAICASNDVAVSMMDGNSGLSLTQEVIDEAVDFRQAMARLYKEFTADGSWFFKPWNKEVVTDPQTGLTYLFALAPKLLTTVQDCWVMHPGESWHGFKDIPDNWSMLDPIKVSILAPGMGEDGELEETGVPAALVTAWLGRHGIVPTRTTDFQIMFLFSMGVTRGKWGTLVNTLCSFKRHYDANTPLAQVMPELVEQYPDTYANMGIHDLGDTMFAWLKENNPGARLNEAYSGLPVAEVTPREAYNAIVDNNVELVSIENLPGRIAANSVIPYPPGIPMLLSGENFGDKNSPQVSYLRSLQSWDHHFPGFEHETEGTEIIDGIYHVMCVKAGGSGGT

SEQ ID NO: 18 - protein, 147 aa, synthetic construct
MVNEVIDINEAVRAYIAQIEGLRAEIGRLDATIATLRQSLATLKSLKTLGEGKTVLVPVGSIAQVEMKVEKMDKVVVSVGQNISAELEYEEALKYIEDEIKKLLTFRLVLEQAIAELYAKIEDLIAEAQQTSEEEKAEEEENEEKAE

SEQ ID NO: 19 - protein, 464 aa, synthetic construct [sequence omitted]

SEQ ID NO: 20 - protein, 375 aa, synthetic construct
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 21 - protein, 51 aa, synthetic construct
MFRIEGLAPKLDPEEMKRKMREDVISSIRNFLIYVALLRVTPFILKKLDSI

SEQ ID NO: 22 - protein, 24 aa, synthetic construct
MVGRNSAIAAGVCGALFIGYCIYF

SEQ ID NO: 23 - protein, 163 aa, synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAGGGSGGTGGGKPIPNPLLGLDST

SEQ ID NO: 24 - protein, 946 aa, synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAGGSGGTGGMIDYTAAGFTLLQGAHLYAPEDRGICDVLVANGKIIAVASNIPSDIVPNCTVVDLSGQILCPGFIDQHVHLIGGGGEAGPTTRTPEVALSRLTEAGVTSVVGLLGTDSISRHPESLLAKTRALNEEGISAWMLTGAYHVPSRTITGSVEKDVAIIDRVIGVKCAISDHRSAAPDVYHLANMAAESRVGGLLGGKPGVTVFHMGDSKKALQPIYDLLENCDVPISKLLPTHVNRNVPLFYQALEFARKGGTIDITSSIDEPVAPAEGIARAVQAGIPLARVTLSSDGNGSQPFFDDEGNLTHIGVAGFETLLETVQVLVKDYDFSISDALRPLTSSVAGFLNLTGKGEILPGNDADLLVMTPELRIEQVYARGKLMVKDGKACVKGTFETAGGSGGTGGSGGTGGSGGTGGSGGTGGKPIPNPLLGLDSTGSGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 25 - protein, 372 aa, synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAGGSGGTGGMSSEKEELRERLVKICVELAKLKGDDTLKAAEAAEEAFRLVVLAAMLAGIDSSEVLELAIRLIKTCVVLAAMEGYDISEACRAAAEAFTRVAMAALRAGITSSLVLKAAIELIKECVLNAAVEGYDISEACRAAAEAFKRVAEAAKRAGITSLETLLRAIEEIRKRVEEAQREGNDISEACRQAAEEFRKKAEELKRRGDVGGGSGGTGGGKPIPNPLLGLDST

SEQ ID NO: 26 - protein, 341 aa, synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAGGSGGTGGTKEERVLLMKVAILAIVAAKKGNTDEVRKALELALLIAKVSGTTEAVKLALEVVARVAIEAARRGNTDAVREALEVALEIARESGTTEAVKLALEVVARVAIEAARRGNTEAVVEALLVALEIAKESGTEEAVRLALEVVKRVSNEALKQGNVDAVKVALEVRKMIEELSGGGSGGTGGGKPIPNPLLGLDST

SEQ ID NO: 27 - protein, 930 aa, synthetic construct
MKVLIVESEFLHQDTWVGNAVERLADALSQQNVTVIKSTSFDDGFAILSSNEAIDCLMFSYQMEHPDEHQNVRQLIGKLHERQQNVPVFLLGDREKALAAMDRDLLELVDEFAWILEDTADFIAGRAVAAMTRYRQQLLPPLFSALMKYSDIHEYSWAAPGHQGGVGFTKTPAGRFYHDYYGENLFRTDMGIERTSLGSLLDHTGAFGESEKYAARVFGADRSWSVVVGTSGSNRTIMQACMTDNDVVVVDRNCHKSIEQGLMLTGAKPVYMVPSRNRYGIIGPIYPQEMQPETLQKKISESPLTKDKAGQKPSYCVVTNCTYDGVCYNAKEAQDLLEKTSDRLHFDEAWYGYARFNPIYADHYAMRGEPGDHNGPTVFATHSTHKLLNALSQASYIHVREGRGAINFSRFNQAYMMHATTSPLYAICASNDVAVSMMDGNSGLSLTQEVIDEAVDFRQAMARLYKEFTADGSWFFKPWNKEVVTDPQTGLTYLFALAPKLLTTVQDCWVMHPGESWHGFKDIPDNWSMLDPIKVSILAPGMGEDGELEETGVPAALVTAWLGRHGIVPTRTTDFQIMFLFSMGVTRGKWGTLVNTLCSFKRHYDANTPLAQVMPELVEQYPDTYANMGIHDLGDTMFAWLKENNPGARLNEAYSGLPVAEVTPREAYNAIVDNNVELVSIENLPGRIAANSVIPYPPGIPMLLSGENFGDKNSPQVSYLRSLQSWDHHFPGFEHETEGTEIIDGIYHVMCVKAGGSGGTGGSGGTGGGKPIPNPLLGLDSTGGSGGTGGMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 28 - protein, 317 aa, synthetic construct
MVNEVIDINEAVRAYIAQIEGLRAEIGRLDATIATLRQSLATLKSLKTLGEGKTVLVPVGSIAQVEMKVEKMDKVVVSVGQNISAELEYEEALKYIEDEIKKLLTFRLVLEQAIAELYAKIEDLIAEAQQTSEEEKAEEEENEEKAEGGSGGTGGGKPIPNPLLGLDSTGGSGGTGGMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 29 - protein, 634 aa, synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAGGSGGTGGMGDVGPRWANWDPSRSGVRDHPGQQSETLPHATFLQKIKFWEVISDEHGIDPTGTYHGDSDLQLDRISVYYNEATGGKYVPRAILVDLEPGTMDSVRSGPFGQIFRPDNFVFGQSGAGNNWAKGHYTEGAELVDSVLDVVWKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQVFDAKNMMAACDPRHGRYLTVAAVFRGRMSMKEVDEQMLNVQNKNSSYFVEWIPNNVKTAVCDIPPRGLKMAVTFIGNSTAIQELFKRISEQFTAMFRRKAFLHWYTGEGMDEMEFTEAESNMNDLVSEYQQYQDATAEEEEDFGEEAEEEAGGSGGTGGGKPIPNPLLGLDST

SEQ ID NO: 30 - protein, 545 aa, synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAGGSGGTGGMDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCFGGSGGTGGGKPIPNPLLGLDST

SEQ ID NO: 31 - protein, 221 aa, synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAGGSGGTGGMFRIEGLAPKLDPEEMKRKMREDVISSIRNFLIYVALLRVTPFILKKLDSIGGSGGTGGGKPIPNPLLGLDST

SEQ ID NO: 32 - protein, 194 aa, synthetic construct
MVGRNSAIAAGVCGALFIGYCIYFGGSGGTGGMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAGGSGGTGGGKPIPNPLLGLDST

SEQ ID NO: 33 - protein, 443 aa, synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSATAULAKQGL

SEQ ID NO: 34 - protein, 1061 aa, synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNGGSGGTGGMIDYTAAGFTLLQGAHLYAPEDRGICDVLVANGKIIAVASNIPSDIVPNCTVVDLSGQILCPGFIDQHVHLIGGGGEAGPTTRTPEVALSRLTEAGVTSVVGLLGTDSISRHPESLLAKTRALNEEGISAWMLTGAYHVPSRTITGSVEKDVAIIDRVIGVKCAISDHRSAAPDVYHLANMAAESRVGGLLGGKPGVTVFHMGDSKKALQPIYDLLENCDVPISKLLPTHVNRNVPLFYQALEFARKGGTIDITSSIDEPVAPAEGIARAVQAGIPLARVTLSSDGNGSQPFFDDEGNLTHIGVAGFETLLETVQVLVKDYDFSISDALRPLTSSVAGFLNLTGKGEILPGNDADLLVMTPELRIEQVYARGKLMVKDGKACVKGTFETAGGSGGTGGSGGTGGSGGTGGSGGTGGKPIPNPLLGLDSTGSGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 35 - protein, 487 aa, synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNGGSGGTGGMSSEKEELRERLVKICVELAKLKGDDTLKAAEAAEEAFRLVVLAAMLAGIDSSEVLELAIRLIKTCVVLAAMEGYDISEACRAAAEAFTRVAMAALRAGITSSLVLKAAIELIKECVLNAAVEGYDISEACRAAAEAFKRVAEAAKRAGITSLETLLRAIEEIRKRVEEAQREGNDISEACRQAAEEFRKKAEELKRRGDVGGGSGGTGGGKPIPNPLLGLDST

SEQ ID NO: 36 - protein, 456 aa, synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNGGSGGTGGTKEERVLLMKVAILAIVAAKKGNTDEVRKALELALLIAKVSGTTEAVKLALEVVARVAIEAARRGNTDAVREALEVALEIARESGTTEAVKLALEVVARVAIEAARRGNTEAVVEALLVALEIAKESGTEEAVRLALEVVKRVSNEALKQGNVDAVKVALEVRKMIEELSGGGSGGTGGGKPIPNPLLGLDST

SEQ ID NO: 37 - protein, 1045 aa, synthetic construct
MKVLIVESEFLHQDTWVGNAVERLADALSQQNVTVIKSTSFDDGFAILSSNEAIDCLMFSYQMEHPDEHQNVRQLIGKLHERQQNVPVFLLGDREKALAAMDRDLLELVDEFAWILEDTADFIAGRAVAAMTRYRQQLLPPLFSALMKYSDIHEYSWAAPGHQGGVGFTKTPAGRFYHDYYGENLFRTDMGIERTSLGSLLDHTGAFGESEKYAARVFGADRSWSVVVGTSGSNRTIMQACMTDNDVVVVDRNCHKSIEQGLMLTGAKPVYMVPSRNRYGIIGPIYPQEMQPETLQKKISESPLTKDKAGQKPSYCVVTNCTYDGVCYNAKEAQDLLEKTSDRLHFDEAWYGYARFNPIYADHYAMRGEPGDHNGPTVFATHSTHKLLNALSQASYIHVREGRGAINFSRFNQAYMMHATTSPLYAICASNDVAVSMMDGNSGLSLTQEVIDEAVDFRQAMARLYKEFTADGSWFFKPWNKEVVTDPQTGLTYLFALAPKLLTTVQDCWVMHPGESWHGFKDIPDNWSMLDPIKVSILAPGMGEDGELEETGVPAALVTAWLGRHGIVPTRTTDFQIMFLFSMGVTRGKWGTLVNTLCSFKRHYDANTPLAQVMPELVEQYPDTYANMGIHDLGDTMFAWLKENNPGARLNEAYSGLPVAEVTPREAYNAIVDNNVELVSIENLPGRIAANSVIPYPPGIPMLLSGENFGDKNSPQVSYLRSLQSWDHHFPGFEHETEGTEIIDGIYHVMCVKAGGSGGTGGSGGTGGGKPIPNPLLGLDSTGGSGGTGGMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKN

SEQ ID NO: 38 - protein, 432 aa, synthetic construct
MVNEVIDINEAVRAYIAQIEGLRAEIGRLDATIATLRQSLATLKSLKTLGEGKTVLVPVGSIAQVEMKVEKMDKVVVSVGQNISAELEYEEALKYIEDEIKKLLTFRLVLEQAIAELYAKIEDLIAEAQQTSEEEKAEEEENEEKAEGGSGGTGGGKPIPNPLLGLDSTGGSGGTGGMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKN

SEQ ID NO: 39 - protein, 749 aa, synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNGGSGGTGGMGDVGPRWANWDPSRSGVRDHPGQQSETLPHATFLQKIKFWEVISDEHGIDPTGTYHGDSDLQLDRISVYYNEATGGKYVPRAILVDLEPGTMDSVRSGPFGQIFRPDNFVFGQSGAGNNWAKGHYTEGAELVDSVLDVVWKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQVFDAKNMMAACDPRHGRYLTVAAVFRGRMSMKEVDEQMLNVQNKNSSYFVEWIPNNVKTAVCDIPPRGLKMAVTFIGNSTAIQELFKRISEQFTAMFRRKAFLHWYTGEGMDEMEFTEAESNMNDLVSEYQQYQDATAEEEEDFGEEAEEEAGGSGGTGGGKPIPNPLLGLDST

SEQ ID NO: 40 - protein, 660 aa, synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNGGSGGTGGMDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCFGGSGGTGGGKPIPNPLLGLDST

SEQ ID NO: 41 - protein, 336 aa, synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNGGSGGTGGMFRIEGLAPKLDPEEMKRKMREDVISSIRNFLIYVALLRVTPFILKKLDSIGGSGGTGGGKPIPNPLLGLDST

SEQ ID NO: 42 - protein, 309 aa, synthetic construct
MVGRNSAIAAGVCGALFIGYCIYFGGSGGTGGMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNGGSGGTGGGKPIPNPLLGLDST

SEQ ID NO: 43 - protein, 279 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHGGGSGGTGGGKPIPNPLLGLDST

SEQ ID NO: 44 - protein, 1062 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHGGSGGTGGMIDYTAAGFTLLQGAHLYAPEDRGICDVLVANGKIIAVASNIPSDIVPNCTVVDLSGQILCPGFIDQHVHLIGGGGEAGPTTRTPEVALSRLTEAGVTSVVGLLGTDSISRHPESLLAKTRALNEEGISAWMLTGAYHVPSRTITGSVEKDVAIIDRVIGVKCAISDHRSAAPDVYHLANMAAESRVGGLLGGKPGVTVFHMGDSKKALQPIYDLLENCDVPISKLLPTHVNRNVPLFYQALEFARKGGTIDITSSIDEPVAPAEGIARAVQAGIPLARVTLSSDGNGSQPFFDDEGNLTHIGVAGFETLLETVQVLVKDYDFSISDALRPLTSSVAGFLNLTGKGEILPGNDADLLVMTPELRIEQVYARGKLMVKDGKACVKGTFETAGGSGGTGGSGGTGGSGGTGGSGGTGGKPIPNPLLGLDSTGSGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 45 - protein, 488 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHGGSGGTGGMSSEKEELRERLVKICVELAKLKGDDTLKAAEAAEEAFRLVVLAAMLAGIDSSEVLELAIRLIKTCVVLAAMEGYDISEACRAAAEAFTRVAMAALRAGITSSLVLKAAIELIKECVLNAAVEGYDISEACRAAAEAFKRVAEAAKRAGITSLETLLRAIEEIRKRVEEAQREGNDISEACRQAAEEFRKKAEELKRRGDVGGGSGGTGGGKPIPNPLLGLDST

SEQ ID NO: 46 - protein, 457 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHGGSGGTGGTKEERVLLMKVAILAIVAAKKGNTDEVRKALELALLIAKVSGTTEAVKLALEVVARVAIEAARRGNTDAVREALEVALEIARESGTTEAVKLALEVVARVAIEAARRGNTEAVVEALLVALEIAKESGTEEAVRLALEVVKRVSNEALKQGNVDAVKVALEVRKMIEELSGGGSGGTGGGKPIPNPLLGLDST

SEQ ID NO: 47 - protein, 1046 aa, synthetic construct
MKVLIVESEFLHQDTWVGNAVERLADALSQQNVTVIKSTSFDDGFAILSSNEAIDCLMFSYQMEHPDEHQNVRQLIGKLHERQQNVPVFLLGDREKALAAMDRDLLELVDEFAWILEDTADFIAGRAVAAMTRYRQQLLPPLFSALMKYSDIHEYSWAAPGHQGGVGFTKTPAGRFYHDYYGENLFRTDMGIERTSLGSLLDHTGAFGESEKYAARVFGADRSWSVVVGTSGSNRTIMQACMTDNDVVVVDRNCHKSIEQGLMLTGAKPVYMVPSRNRYGIIGPIYPQEMQPETLQKKISESPLTKDKAGQKPSYCVVTNCTYDGVCYNAKEAQDLLEKTSDRLHFDEAWYGYARFNPIYADHYAMRGEPGDHNGPTVFATHSTHKLLNALSQASYIHVREGRGAINFSRFNQAYMMHATTSPLYAICASNDVAVSMMDGNSGLSLTQEVIDEAVDFRQAMARLYKEFTADGSWFFKPWNKEVVTDPQTGLTYLFALAPKLLTTVQDCWVMHPGESWHGFKDIPDNWSMLDPIKVSILAPGMGEDGELEETGVPAALVTAWLGRHGIVPTRTTDFQIMFLFSMGVTRGKWGTLVNTLCSFKRHYDANTPLAQVMPELVEQYPDTYANMGIHDLGDTMFAWLKENNPGARLNEAYSGLPVAEVTPREAYNAIVDNNVELVSIENLPGRIAANSVIPYPPGIPMLLSGENFGDKNSPQVSYLRSLQSWDHHFPGFEHETEGTEIIDGIYHVMCVKAGGSGGTGGSGGTGGGKPIPNPLLGLDSTGGSGGTGGMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVH

SEQ ID NO: 48 - protein, 433 aa, synthetic construct
MVNEVIDINEAVRAYIAQIEGLRAEIGRLDATIATLRQSLATLKSLKTLGEGKTVLVPVGSIAQVEMKVEKMDKVVVSVGQNISAELEYEEALKYIEDEIKKLLTFRLVLEQAIAELYAKIEDLIAEAQQTSEEEKAEEEENEEKAEGGSGGTGGGKPIPNPLLGLDSTGGSGGTGGMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVH

SEQ ID NO: 49 - protein, 750 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHGGSGGTGGMGDVGPRWANWDPSRSGVRDHPGQQSETLPHATFLQKIKFWEVISDEHGIDPTGTYHGDSDLQLDRISVYYNEATGGKYVPRAILVDLEPGTMDSVRSGPFGQIFRPDNFVFGQSGAGNNWAKGHYTEGAELVDSVLDVVWKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQVFDAKNMMAACDPRHGRYLTVAAVFRGRMSMKEVDEQMLNVQNKNSSYFVEWIPNNVKTAVCDIPPRGLKMAVTFIGNSTAIQELFKRISEQFTAMFRRKAFLHWYTGEGMDEMEFTEAESNMNDLVSEYQQYQDATAEEEEDFGEEAEEEAGGSGGTGGGKPIPNPLLGLDST

SEQ ID NO: 50 - protein, 661 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHGGSGGTGGMDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCFGGSGGTGGGKPIPNPLLGLDST

SEQ ID NO: 51 - protein, 337 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHGGSGGTGGMFRIEGLAPKLDPEEMKRKMREDVISSIRNFLIYVALLRVTPFILKKLDSIGGSGGTGGGKPIPNPLLGLDST

SEQ ID NO: 52 - protein, 310 aa, synthetic construct
MVGRNSAIAAGVCGALFIGYCIYFGGSGGTGGMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHGGSGGTGGGKPIPNPLLGLDST

SEQ ID NO: 53 - protein, 8 aa, synthetic construct
GGSGGTGG

SEQ ID NO: 54 - protein, 14 aa, synthetic construct
GKPIPNPLLGLDST

(19) United States
(12) Patent Application Publication
(10) Pub. No.: US 2026/0265322 A1
(43) Pub. Date: Sep. 10, 2026

(54) SELF-ASSEMBLING SYSTEMS FOR MODELING INTRACELLULAR PROTEIN AGGREGATION

(71) Applicant: California Institute of Technology, Pasadena, CA (US)

(72) Inventors: Viviana Gradinaru, Pasadena, CA (US); Yujie Fan, Pasadena, CA (US)

(21) Appl. No.: 19/561,166

(22) Filed: Mar. 9, 2026

Related U.S. Application Data
(60) Provisional application No. 63/769,655, filed on Mar. 10, 2025.

(51) Int. Cl. C07K 14/47; C12N 15/62; C12N 15/86; G01N 33/50

(52) U.S. Cl. CPC C07K 14/4711; C12N 15/62; C12N 15/86; G01N 33/5032; G01N 33/5091; C07K 2319/715; C07K 2319/735; C12N 2750/14143; C12N 2750/14145; C12N 2800/40; G01N 2500/10

(57) ABSTRACT

Disclosed herein include polynucleotides, expression vectors and compositions enabling expression of a self-assembling proteopathic fusion protein comprising a self-assembling domain fused to a proteopathic polypeptide associated with a proteinopathy. The self-assembling proteopathic fusion protein is capable of forming intracellular protein aggregates that enable robust recapitulation of disease pathology. Provided herein also include methods of using the self-assembling systems disclosed herein to induce proteinopathies (e.g., neurodegenerative diseases) and to screen therapeutic compounds for the treatment of proteinopathies.

### RELATED APPLICATIONS

This application claims the benefit under 35 U.S.C. § 119(e) of U.S. Provisional Patent Application Ser. No. 63/769,655, filed on Mar. 10, 2025, the content of this related application is incorporated herein by reference in its entirety for all purposes.

### REFERENCE TO SEQUENCE LISTING

The present application is being filed along with a Sequence Listing in electronic format. The Sequence Listing is provided as a file entitled 30KJ-810023-US_SeqList, created Feb. 27, 2026, which is 71,097 bytes in size. The information in the electronic format of the Sequence Listing is incorporated herein by reference in its entirety.

### BACKGROUND

### Field

The present disclosure relates generally to the field of polynucleotide expression.

### Description of the Related Art

Protein aggregation is a pathological hallmark of numerous neurodegenerative disorders, including Parkinson's disease (PD), Alzheimer's disease (AD), and amyotrophic lateral sclerosis (ALS). In these conditions, misfolded proteins form intracellular or extracellular inclusions that disrupt cellular homeostasis, impair organelle function, propagate through neural circuits and eventually lead to neuronal loss and behavioral changes. Despite their shared etiology of protein aggregation, different neurodegenerative diseases involve distinct aggregating proteins, affect different brain regions, exhibit unique patterns of cell-type vulnerability, and lead to divergent clinical outcomes. It remains challenging to understand which forms of aggregates are toxic, how aggregates spread between cells and drive neurotoxicity, and why certain neurons are more vulnerable to specific protein aggregates than others.

### SUMMARY

Provided herein includes a polynucleotide encoding a self-assembling proteopathic fusion protein, comprising: a first nucleic acid unit encoding a proteopathic polypeptide and a second nucleic acid unit encoding a self-assembling domain.

Provided herein also includes an expression vector. The expression vector can comprise: one or more first promoters operably connected to a first polynucleotide comprising a first nucleic acid unit encoding a proteopathic polypeptide and a second nucleic acid unit encoding a self-assembling domain, wherein the first polynucleotide, upon expression in a cell, encodes a self-assembling proteopathic fusion protein comprising the proteopathic polypeptide connected to the self-assembling domain and capable of forming intracellular protein aggregates in the cell.

In some embodiments, the proteopathic polypeptide is associated with a neurodegenerative disease, optionally, the neurodegenerative disease is selected from the group consisting of: Alzheimer's disease, Parkinson's disease, amyotrophic lateral sclerosis (ALS), Huntington's disease, frontotemporal dementia (FTD), chronic traumatic encephalopathy (CTE), multiple system atrophy (MSA), and Lewy body dementia (LBD). The proteopathic polypeptide can be selected from the group consisting of: alpha synuclein polypeptide, Tau, TDP-43, an Aβ precursor protein, an Aβ peptide, a prion, serum amyloid A, amyloid β-protein, transthyretin, cystatin C, beta-2-microglobulin, apolipoprotein AI, apolipoprotein AII, gelsolin, amylin, calcitonin, atrial natriuretic factor, lysozyme, insulin, fibrinogen α-A chain, superoxide dismutase 1, huntingtin, an androgen receptor, atrophin, an ataxin, immunoglobulin light chain, neuroserpin, superoxide dismutase 1, FUS, EWS RNA binding protein 1, and a TATA box-binding protein. In some embodiments, the proteopathic polypeptide is Tau, TDP-43, alpha synuclein, huntingtin, amyloid β-protein, or a portion thereof. In some embodiments, the proteopathic polypeptide comprises an amino acid sequence set forth in SEQ ID Nos: 1-13 or an amino acid sequence having at least 80%, at least 85%, at least 90%, at least 95%, at least 96%, at least 97%, at least 98%, at least 99%, or 100% sequence identity to any one of SEQ ID Nos: 1-13.

In some embodiments, the self-assembling domain is selected from the group consisting of: an actin protein, a tubulin protein, a cytoskeletal filament, gamma-prefoldin, viral capsid protein, cage protein, proteins driving phase separation, engineered protein with assembly-gain-of-function mutant, or a variant or a fragment thereof. Optionally, the viral capsid protein is HIV-1 capsid protein or hepatitis B core antigen; the cage protein is ferritin; the protein driving phase separation is FUS or hnRNPA1; the engineered protein with assembly-gain-of-function mutation is isoaspartyl dipeptidase with E239Y mutation, ketopantoate hydroxymethyl transferase with E158L mutation, acid induced arginine decarboxylase with K491L, D494L and D497L mutation, E. coli AsnC with K126Y and D1313Y mutation. In some embodiments, the self-assembling domain has an amino acid sequence set forth in SEQ ID Nos: 14-22 or an amino acid sequence having at least 80%, at least 85%, at least 90%, at least 95%, at least 96%, at least 97%, at least 98%, at least 99%, or 100% sequence identity to any one of SEQ ID Nos: 14-22.

In some embodiments, the proteopathic polypeptide is connected to the self-assembling domain via a linker. The self-assembling proteopathic fusion protein can further comprise a molecular tag.

In some embodiments, the first promoter is an inducible promoter or a cell- or tissue-specific promoter, optionally, the first promoter is a neuron-specific promoter. In some embodiments, the first promoter is an inducible promoter, and the expression vector further comprises a second promoter operably linked to a second polynucleotide encoding a transactivator. In the presence of the transactivator and a transactivator-binding compound, the first promoter is capable of inducing transcription of the first polynucleotide to generate the self-assembling proteopathic fusion protein. The first promoter can comprise one or more copies of a transactivator recognition sequence the transactivator is capable of binding to induce transcription, wherein the transactivator is incapable of binding the transactivator recognition sequence in the absence of the transactivator-binding compound. The first promoter can comprise a tetracycline response element (TRE), the transactivator can comprise reverse tetracycline-controlled transactivator (rtTA), and the transactivator-binding compound can comprise tetracycline, doxycycline or a derivative thereof. In some embodiments, the second promoter is cell- or tissue-specific promoter, optionally, the second promoter is a neuron-specific promoter.

The expression vector can be a viral vector. The viral vector can be an adeno-associated virus (AAV), optionally the AAV vector comprises AAV1, AAV2, AAV3, AAV4, AAV5, AAV6, AAV7, AAV8, AAV9, derivatives thereof, or any combination thereof. The AAV vector can comprise an AAV9 variant engineered for systemic delivery, optionally AAV-PHP.B, AAV-PHP.eB, or AAV-PHP.S. In some embodiments, the AAV comprises a targeting peptide that targets the AAV to the nervous system of a subject. In some embodiments, the viral vector is encapsidated in a viral particle.

Provided herein also includes an expression system. The expression system can comprise: a first expression vector comprising a first promoter operably connected to a first polynucleotide comprising a first nucleic acid unit encoding a proteopathic polypeptide and a second nucleic acid unit encoding a self-assembling domain; and a second expression vector comprising a second promoter operably connected to a second polynucleotide encoding a transactivator, wherein the first promoter is an inducible promoter capable of inducing transcription of the first polynucleotide in the presence of the transactivator and a transactivator-binding compound, and wherein the first polynucleotide, upon expression in a cell, encodes a self-assembling proteopathic fusion protein comprising the proteopathic polypeptide connected to the self-assembling domain and capable of forming intracellular protein aggregates in the cell.

The first promoter can comprise one or more copies of a transactivator recognition sequence the transactivator is capable of binding to induce transcription, wherein the transactivator is incapable of binding the transactivator recognition sequence in the absence of the transactivator-binding compound. The first promoter can comprise a tetracycline response element (TRE), the transactivator comprises reverse tetracycline-controlled transactivator (rtTA), and the transactivator-binding compound comprises tetracycline, doxycycline or a derivative thereof. The second promoter can be a cell- or tissue-specific promoter, optionally, the second promoter can be a neuron-specific promoter.

Provided herein also includes a composition comprising the polynucleotide disclosed herein. Provided herein also includes a self-assembling proteopathic fusion protein generated by the polynucleotide or the expression vector or the expression system disclosed herein. Provided herein also includes a self-assembling proteopathic fusion protein. The self-assembling proteopathic fusion protein can comprise a first nucleic acid unit encoding a proteopathic polypeptide and a second nucleic acid unit encoding a self-assembling domain.

Provided herein also includes a method of inducing a proteinopathy in a cell. The method can comprise: introducing into a cell the polynucleotide or the expression vector or the expression system disclosed herein; and expressing the self-assembling proteopathic fusion protein capable of forming intracellular protein aggregates in the cell, thereby inducing a proteinopathy associated with the proteopathic polypeptide.

Provided herein also includes a method of identifying a candidate compound for the treatment of a proteinopathy. The method can comprise introducing into a cell the polynucleotide or the expression vector or the expression system disclosed herein; expressing the self-assembling proteopathic fusion protein capable of forming intracellular protein aggregates in the cell, wherein the proteopathic polypeptide is associated with a proteinopathy; administering to the cell with a compound of interest; and determining the effect of the compound of interest on the proteinopathy.

In some embodiments, the proteinopathy is a neurodegenerative disease. The neurodegenerative disease can be Alzheimer's disease, Parkinson's disease, amyotrophic lateral sclerosis, Huntington's disease, frontotemporal dementia, chronic traumatic encephalopathy, multiple system atrophy, and Lewy body dementia. In some embodiments, determining the effect of the compound of interest on the proteinopathy comprises detecting pathological features of the proteinopathy. The method can further comprise selecting the compound of interest as a candidate compound if a decrease in the number and/or size of intracellular protein aggregates or an improvement in the pathological features, the phenotype, symptoms, and/or severity of the proteinopathy is observed. The method can be an in vitro method, an ex vivo method, or an in vivo method. The cell can be in vitro, in vivo, or ex vivo.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1J depicts non-limiting exemplary data related to an exemplary self-assembling system that induces intracellular protein aggregates and enables tunable proteiopathy. FIG. 1A: Design of self-assembling synuclein (SAS) system. SAS consists of αSyn fused with a self-assembling protein. In HEK cells, rtTA expression is driven by the EF1α promoter. Upon doxycycline (dox) induction, SAS forms αSyn aggregates intracellularly. FIG. 1B: Screening of SAS constructs in HEK cells. Compared to ctrl (rtTA-transfected only), SNCA overexpression (SNCA OE) or self-assembling protein (SAP) alone, SAS3 induces αSyn aggregation and strong αSyn S129 phosphorylation after 3 days of dox treatment in transfected HEK cells. FIGS. 1C-1D: Western blot analysis of total expression (V5 tag, FIG. 1C) and pS129-αSyn (FIG. 1D), reveals SAS3-induced aggregates trigger markedly stronger αSyn phosphorylation than SNCA OE, suggesting enhanced pathogenic potential. n=3 biological replicates. FIG. 1E: Representative images of SAS-induced aggregates at varing dox concentration. FIGS. 1F-1G: Quantification of aggregate number per cell (FIG. 1F) and average aggregate size (FIG. 1G) for different dox concentrations, demonstrating tunability of the system. n=9 biological replicates. FIG. 1H: Schematic of modular design for self-assembling Tau (SA-Tau) and self-assembling TDP-43 (SA-TDP), in which human Tau (isoform 2N4R) or TDP-43 is fused with the same self-assembling protein used in SAS3. A GFP tag is used to detect aggregates. FIG. 1I: Immunostaining of HEK cells transduced with SA-Tau-GFP and stained for pTau (AT8) after 3 days of dox induction shows robust Tau aggregation and hyper phosphorylation. FIG. 1J: Immunostaining of SA-TDP-GFP-expressing HEK cells after 3 days of dox induction reveals cytoplasmic TDP-43 inclusions with strong pTDP-43 signal. Scale bars equal 20 μm. Data are presented as mean±SEM.

FIG. 2 depicts non-limiting exemplary data demonstrating that self-assembling αSyn, Tau, and TDP-43 aggregates recapitulate distinct, protein-specific histopathology in neurons. (Panel A) Experimental design for testing the self-assembling system in mouse primary neurons, which is delivered by AAV9-X1.1. In primary neurons, rtTA expression is driven by the hSyn promoter. (Panel B) Immunostaining of V5 tag, pS129-αSyn, and MAP2 in transduced primary neurons after 3 days of dox treatment, showing robust αSyn aggregation and pS129-αSyn in both the soma and neurites of neurons, as well as neurite retraction. (Panel C) Quantification of average aggregate size induced by SAS3 in mouse primary neurons. (Panel D) Average number of aggregates per cell. (Panel E) Quantification of total neurite length per cell. n=4 biological replicates. ****p<0.0001. (Panel F) Immunostaining of GFP, pTau (AT8 antibody), and MAP2 in SA-Tau-transduced neurons after 3 days of dox treatment, reveals robust Tau aggregation, hyper pTau, and pronounced neurite degeneration. Enlarged images demonstrate that SA-Tau and SASs form distinct aggregate structures, despite the use of the same self-assembling protein. (Panel G) Immunostaining for amyloid beta in primary neurons expressing SA-Tau indicates Tau tangles promote the production of extracellular amyloid beta (arrowhead). (Panel H) Representative images of neurons expressing SA-TDP after 3 days of dox treatment show cytoplasmic TDP-43 inclusions with strong pTDP-43. Compared with equivalent expression of SAS and SA-Tau, SA-TDP induces milder neurodegeneration. (Panel I) Immunostaining for total TDP-43 reveals translocation of endogenous TDP-43 from nucleus to cytoplasm and aggregation (arrowhead). Scale bar equals 50 μm in panels B, F, H, and 10 μm in panels G, I. Data are presented as mean±SEM; statistical analysis was performed using ANOVA with Dunnett's test.

FIGS. 3A-3K depict non-limiting exemplary data related to distinct organelle interactions and pathological signatures of αSyn, Tau, and TDP-43 aggregates in human brain organoids revealed by exemplary self-assembling systems. FIG. 3A: Schematic of generating human cortical organoids from hESCs. FIG. 3B: Immunostaining of fluorescently-labeled PFFs or SAS3 (V5 tag) and pS129-αSyn in hESC-derived cortical organoids. Unlike PFFs, SAS3 can penetrate and induce PD-like pathology through the entire organoid. Scale bar equals 200 μm and 50 μm in enlargement. FIG. 3C: Z-scanning confocal imaging of organoids stained for V5 tag and the mitochondrial marker TOMM20. SAS3-induced aggregates colocalize with mitochondria (arrowhead), shown by XY (main panel), XZ (bottom panel) and YZ (right panel) projections. Scale bar equals 10 μm. FIGS. 3D-F: Organoids stained for ubiquitin and p62/SQSTM1 or with Nile red reveal that SAS3-induced aggregates are complex inclusions containing αSyn and other proteins, similar to LBs found in human PD patients. FIG. 3D: Ubiquitin staining confirms that proteins in SAS3-induced aggregates are highly ubiquitinated. FIG. 3E: Accumulation of p62 in and around the aggregates indicates elevated autophagy. FIG. 3F: Nile red staining demonstrates enrichment of lipids in SAS-induced aggregates. FIG. 3G: Mitochondrial marker TOMM20 accumulates surrounding SA-Tau aggregates (arrowhead). FIGS. 3H-3I: SA-Tau-induced aggregates are highly ubiquitinated (FIG. 3H) and co-localize with neurofilament light chain (NfL) (FIG. 3I). FIG. 3J: In contrast, SA-TDP-induced aggregates show little to no co-localization with mitochondria (arrowhead), indicating a distinct aggregate composition compared to SAS and SA-Tau. FIG. 3K: Schematic comparison of SAS, SA-Tau and SA-TDP aggregates, highlighting differences in aggregate morphology, interaction patterns with organelles, and associated neurotoxicity. Scale bar equals 5 μm in FIGS. 3D-3J.

FIGS. 4A-4H depict non-limiting exemplary data showing that SAS forms LB-like inclusions and nucleate aggregation of endogenous αSyn, triggering neuronal inflammation and death. FIG. 4A: Experimental design for testing whether SAS3 can nucleate secondary aggregation of endogenous αSyn in primary neurons carrying the SNCA-GFP reporter. FIGS. 4B-4C: Immunostaining after 36 hours of dox treatment shows SAS3-induced aggregates contain endogenous αSyn, as indicated by GFP signal. Scale bar equals 50 μm in (FIG. 4B) and 2 μm in (FIG. 4C). FIG. 4D: Principal coordinate analysis (PCoA) shows a clear separation of transcriptomic profiles of SAS3 organoids transduced with hSyn-rtTA and TRE-SAS3 using AAV9-X1.1 and control organoids transduced with only hSyn-rtTA. n=3 biological replicates. FIG. 4E: Volcano plot of DEGs with FDR <0.05 and |log 2 fold change (FC)|>1. Genes belonging to the TNFα pathway are highlighted in magenta, p53 pathway in cyan, and both in black. FIG. 4F: Pathway analysis reveals that SAS3-treated organoids contain significantly upregulated Gene Ontology (GO) pathways associated with apoptosis and inflammation. FIG. 4G Immunostaining for the astrocyte marker GFAP reveals astrocyte activation surrounding SAS3-expressing neurons and active engagement with protein inclusions. Scale bar equals 50 μm in overview and 10 μm in enlargement. FIG. 4H: Top 25 up- and downregulated genes.

FIGS. 5A-5M depict non-limiting exemplary data showing that SAS induces PD-relevant histopathology and behavioral deficits in mice. FIG. 5A: Experimental design for brain-wide delivery of SAS3 in WT C57BL/6J mice through retro-orbital (RO) injection of AAV-PHP.eB. Behavioral assays were performed every other week and histology at week 8. FIG. 5B: After 4 weeks of dox treatment, SAS3-treated mice exhibited progressively decreased movement in the open field test compared with other groups. n=5 mice per group. FIG. 5C: Immunostaining of the V5 tag and TH in the substantia nigra, showing aggregates in both the substantia nigra pars compacta (SNpc) and substantia nigra reticulata (SNr), and a decreased number of TH+ neurons in the SNpc. Scale bar equals 200 μm and 50 μm in enlargements. FIG. 5D: Representative images of dopaminergic neuron projections into the striatum. Scale bar equals 500 μm. FIG. 5E: Quantification of striatal TH intensity. n=3 mice. FIG. 5F: Immunostaining of the V5 tag and pS129-αSyn in the olfactory bulb, showing aggregates and strong αSyn phosphorylation. Scale bar equals 50 μm and 20 μm in enlargement. FIG. 5G: Immunostaining for the V5 tag and pS129-αSyn in the olfactory bulb of SAS3-treated mice after proteinase K treatment, showing resistance of aggregates to degradation. Scale bar equals 20 μm. FIG. 5H: Experimental design for selectively delivering SAS3 into dopaminergic neurons. Cre-dependent SAS3 (DIO-SAS3) was RO injected into TH-Cre mice using AAV-PHP.eB. Behavioral assays and histology were performed at 4, 8, and 12 weeks. FIG. 5I: After 4 weeks of dox treatment, SAS3-treated mice exhibited significant motor impairments in the beam crossing assay, displaying more slips than controls. n=5 mice per group. FIG. 5J: Immunostaining of the V5 tag and TH in the substantia nigra (SN) region, with enlarged images of the ventral tegmental area (VTA) and SNpc. FIG. 5K: Immunostaining of the V5 tag and TH in the striatum, suggesting progressive accumulation of aggregates and potential spreading along the nigrostriatal pathway. Scale bar equals 200 μm and 50 μm in enlargements in FIGS. 5J, 5K. FIGS. 5L-5M) Immunostaining of ubiquitin (FIG. 5L) and p62/SQSTM1 (FIG. 5M) confirms that SAS3-induced aggregates are similar to LBs both in morphology and composition. Scale bars equal 2 μm. Data are presented as mean±SEM; statistical analysis was performed using the ANNOVA with multiple comparison correction post-hoc tests in (FIG. 5B) and unpaired t test in (FIGS. 5E&5I); *p<0.05, **p<0.01.

FIGS. 6A-6G depict non-limiting exemplary data showing that SAS activates microglia and triggers immune response in mice. FIG. 6A: PCoA of transcriptomic data shows separation between SAS3-treated mice (transduced with hSyn-rtTA and TRE-SAS3 using AAV-PHP.eB) and controls (transduced with only hSyn-rtTA). n=3 biological replicates. FIG. 6B: Volcano plot of DEGs with FDR <0.05 and |log 2 fold change (FC)|>1. Genes belonging to the TNFα pathway are highlighted in magenta, IFN-7 pathway in cyan, and both in black. FIG. 6C: Top 10 significantly altered GO pathways in SAS3-transduced mice indicate upregulation of pathways involved in inflammation. FIG. 6D: Top 25 up- and downregulated genes. FIG. 6E: Quantification of upregulation of representative genes of the inflammation-related IFN-7, TNF-α, TLR, and interleukin pathways in SAS3-treated mice compared to controls. FIG. 6F: Quantification of upregulation of representative genes involved in microglia activation. FIG. 6G: Immunostaining of GFAP and IBA1 in the olfactory bulb, showing activated microglia clustered around SAS-induced aggregates. Scale bar equals 50 μm. Data are present as mean±SEM.

FIGS. 7A-7F depict non-limiting exemplary data related to screening of SAS constructs in HEK cells and tunability of the SAS system. FIG. 7A: Screening in HEK cells to identify SAS constructs that form αSyn aggregates and induce substantial Ser129 phosphorylation of αSyn. FIGS. 7B-7C: Quantification of western blot for total expression (V5 tag) and pS129-αSyn show that although SAS3 expression is lower than SNCA overexpression (SNCA OE), it induces 1000-fold greater phosphorylation, indicating that the enhanced pS129-αSyn signal is due to aggregation rather than protein level. n=3 biological replicates. FIG. 7D: Design of SAS construct with GFP reporter. FIG. 7E: Immunostaining of pS129-αSyn with GFP reporter after 3 days of dox induction, demonstrating that the GFP tag does not alter SAS behavior. FIG. 7F: Immunostaining of V5 tag and pS129-αSyn after 3, 5, and 7 days of dox-induced SAS3 expression, demonstrating that aggregate size and level of pS129-αSyn increase with induction time. Scale bars equal 20 μm. Data are presented as mean±SEM.

FIG. 8 depicts non-limiting exemplary data showing that SAS induces αSyn aggregation and neurite retraction in hESC-derived neurons. Immunostaining of V5 tag and pS129-αSyn in transduced hESC-derived human cortical neurons after 3 days of dox treatment shows that SAS3 induces αSyn aggregation, strong αSyn phosphorylation, and neurite retraction. Scale bar equals 50 μm.

FIG. 9 depicts non-limiting exemplary data revealing distinct organelle interactions and pathological signatures of αSyn, Tau, and TDP-43 aggregates. (Panels A-B) Immunostaining of V5 tag and pS129-αSyn in transduced human cortical organoids after 3 days of dox treatment. Compared to SNCA OE and SAP alone, only SAS3 induces αSyn aggregation, intense αSyn S129 phosphorylation, and neuron degeneration. Scale bar equals 200 μm and 50 μm in enlargement. (Panel C) Z-stack scanning of organoids stained for V5 tag and the mitochondrial marker TOMM20, showing that SNCA OE and SAP construct-produced proteins do not colocalize with mitochondria, shown by XY (main), XZ (bottom panel) and YZ (right panel) projections. (Panel D) Immunostaining of GFP, pTau (AT8 antibody), and MAP2 in human brain organoids shows robust Tau aggregation and pTau, as well as severe neuron degeneration. (Panel E) Immunostaining for amyloid beta in human brain organoids expressing SA-Tau reveals Tau tangles promote the production of extracellular amyloid beta. (Panel F) SA-Tau expressing cells exhibited elevated accumulation of p62 signal. (Panel G) Representative images of human brain organoids expressing SA-TDP show cytoplasmic TDP-43 inclusions with strong pTDP-43 (arrowheads). Compared with SAS and SA-Tau, human brain organoids expressing SA-TDP only shows mild neurodegeneration indicated by MAP2 staining. (Panel H) Ubiquitin staining shows SA-TDP-induced aggregates are not ubiquitinated, unlike αSyn or Tau aggregates. (Panel I) Accumulation of p62 in SA-TDP expressing cells indicates elevated autophagy. (Panel J) SA-TDP-induced aggregates do not colocalize with neurofilament light chain (NfL). Scale bar equals m in Panels B-J.

FIG. 10 depicts non-limiting exemplary data showing that SAS forms LB-like inclusions and nucleates aggregation of endogenous αSyn, triggering neuronal inflammation and death. (Panel A) Organoids stained with thioflavin S demonstrate the presence of β-sheet structures within and around SAS-induced aggregates. Scale bar equals 2 μm. (Panel B) Organoids stained for V5 tag and pS129-αSyn reveals the presence of V5-negative and pS129-αSyn-positive aggregates (arrowheads), indicating the formation of endogenous αSyn aggregates. Scale bar equals 5 μm. (Panel C) Immunostaining after 36 and 72 hours of dox treatment indicates that neurons expressing SAS3 display degeneration at 36 hours which further worsens by 72 hours. Scale bar equals 100 μm. (Panel D) Volcano plot of transcriptomic data labeling all DEGs with FDR <0.05 and |log 2 fold change (FC)|>1. Genes upregulated in SAS-treated organoids are shown in red and those downregulated in blue. (Panel E) Pathway analysis of DEGs using different databases reveals that SAS3-treated organoids are characterized by significant up-regulation of pathways associated with apoptosis, inflammation, and mitochondrial changes.

FIGS. 11A-11B depict non-limiting exemplary data showing that systemic SAS induces PD-relevant histopathology. FIG. 11A: Immunostaining for the V5 tag and pS129-αSyn in the olfactory bulb of C57BL/6J mice, showing that SNCA OE and SAP-alone constructs do not induce strong αSyn phosphorylation in olfactory bulb. Scale bar equals 200 μm and 50 μm in enlargement. FIG. 11B: Immunostaining for the V5 tag and pS129-αSyn in the hippocampus and cortex of SAS3-treated or control mice, showing aggregates and strong αSyn phosphorylation in neurons induced by SAS3. Scale bar equals 1 mm and 100 μm in enlargement.

FIG. 12 depicts non-limiting exemplary data showing that dopaminergic neuron-targeted SAS recapitulates PD progression. (Panel A) After 4 weeks of dox treatment, DA neuron-targeted SAS3 mice exhibited significant motor impairments in the beam crossing assay, requiring more time to cross the beam. (Panel B) Immunostaining for the V5 tag and TH in the SN region of SAS3-treated TH-Cre mice at 4 weeks and 8 weeks, with enlarged images of the ventral tegmental area (VTA, area 1), SNpc (area 2), and non-TH+ region (area 3). Scale bars equal 150 μm and 50 μm in enlargements. (Panel C) Immunostaining for the V5 tag and TH in the striatum of SAS3-treated TH-Cre mice at 4 weeks and 8 weeks, indicating progressive aggregate accumulation and potential spreading along the nigrostriatal pathway. Scale bar equals 150 μm and 50 μm in enlargement. (Panel D) Immunostaining for V5, pS129-αSyn, and TH in the SNpc reveals that not only SAS3-expressed proteins (arrows) but also endogenous αsyn (arrowheads) formed aggregates and became phosphorylated. Scale bar equals 50 μm. Data are presented as mean±SEM; statistical analysis was performed using unpaired t test; *p<0.05.

FIGS. 13A-13B depict non-limiting exemplary data showing SAS activates microglia and triggers immune response in mice. FIG. 13A: Most significantly differentially regulated GO pathways, revealing that SAS3-transduced mice show significant up-regulation of pathways associated with inflammation and immune response. FIG. 13B: Immunostaining for GFAP and IBA1 in the hippocampus, showing activated microglia clustered around SAS-induced aggregates. Scale bar equals 50 μm.

FIG. 14 depicts schematics of self-assembling system for intracellular protein aggregation (top panel), protein-specific disease pathology triggered by different self-assembling proteopathic proteins (middle panel), and self-assembling α-Syn (SAS) for Parkinsonian pathology.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include amnion cell models and cells, biomarkers and/or gene signatures identified in various amnion cell types, and related methods, compositions and kits for therapeutic, diagnostic, or screening uses.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Ranges and values may be expressed herein as from “about” one particular value, and/or to “about” another particular value. When such a range is expressed, also specifically contemplated and considered disclosed is the range from the one particular value and/or to the other particular value unless the context specifically indicates otherwise. All of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed. As used herein, the term “about” and the like, when used in the context of a value, generally means plus or minus 10% of the value stated. For example, about 0.5 would include 0.45 and 0.55, about 10 would include 9 to 11, about 1000 would include 900 to 1100.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

Unless specified otherwise, the left-hand end of any single-stranded polynucleotide sequence discussed herein is the 5′ end; the left-hand direction of double-stranded polynucleotide sequences is referred to as the 5′ direction.

The term “naturally occurring” as used herein refers to materials which are found in nature or a form of the materials that is found in nature.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

As used herein, “sequence identity” or “identity” in the context of two nucleic acid or polypeptide sequences makes reference to the nucleotide bases or residues in the two sequences that are the same when aligned for maximum correspondence over a specified comparison window. Methods of alignment of sequences for comparison are well known in the art. Various programs and alignment algorithms are described in. Smith & Waterrnan, Adv. Appl. Math. 2:482, 1981, Needleman & Wunsch, J. MoL Biol. 48.443, 1970; Pearson & Lipmian, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; Pearson et al., Meth. Mol. Bio. 24:307-31, 1994; and Altschul et al., J. Mol. Biol. 215:403-10, 1990, the content of each of which is incorporated herein in its entirety.

When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule. A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The phrase “substantially identical,” in the context of two nucleic acids or polypeptides (e.g., nucleic acids encoding a biosensor or a portion thereof, or the amino acid sequence of a biosensor or a portion thereof) refers to two or more sequences or subsequences that have at least about 60%, about 80%, about 90-95%, about 98%, about 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, the “substantial identity” exists over a region of the sequences that is at least about 50 residues in length, more preferably over a region of at least about 100 residues, or over the full length of the two sequences to be compared.

As used herein, the term “variant” refers to a polynucleotide or polypeptide having a sequence substantially similar or identical to a reference (e.g., the parent) polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

As used herein, the term “fusion protein” refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a recombinase. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein can be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

As used herein, the term “vector” refers to a polynucleotide construct, typically a plasmid or a virus, used to transmit genetic material to a host cell. Vectors can be, for example, viruses, plasmids, cosmids, or phage. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

The term “construct,” as used herein, refers to a recombinant nucleic acid that has been generated for the purpose of the expression of a specific nucleotide sequence(s), or that is to be used in the construction of other recombinant nucleotide sequences.

The term “regulatory element” and “expression control element” are used interchangeably and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “enhancer” refers to a type of regulatory element that can increase the efficiency of transcription, regardless of the distance or orientation of the enhancer relative to the start site of transcription.

As used herein, the term “plasmid” refers to a nucleic acid that can be used to replicate recombinant DNA sequences within a host organism. The sequence can be a double stranded DNA.

As used herein, the term “element” refers to a separate or distinct part of something, for example, a nucleic acid sequence with a separate function within a longer nucleic acid sequence. The terms “transcription regulatory element” and “expression control element” are used to refer to nucleic acid molecules that can influence the expression (including at the transcription and/or translation level) of an operably linked coding sequence in a specific host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell. The promoter can be a specific promoter, e.g., cell type-specific and/or tissue-specific. The promoter can be constituent or inducible (e.g., by chemical agent, biological agent, temperature, and/or pH).

The term “transform” or “transfect” refers to the process of introducing an exogenous DNA inside a cell. The transformed DNA may or may not be integrated (covalently linked) into the genome of the cell. In prokaryotes, yeast, and mammalian cells for example, the transforming DNA may be maintained on an episomal element such as a plasmid. A “clone” is a population of cells derived from a single cell or common ancestor by mitosis. A “cell line” is a clone of a primary cell that is capable of stable growth in vitro for many generations. The nucleic acid may be in the form of naked DNA or RNA, associated with various proteins or the nucleic acid may be incorporated into a vector.

The term “AAV” or “adeno-associated virus” refers to a Dependoparvovirus within the Parvoviridae genus of viruses. For example, the AAV can be an AAV derived from a naturally occurring “wild-type” virus, an AAV derived from a rAAV genome packaged into a capsid derived from capsid proteins encoded by a naturally occurring cap gene and/or a rAAV genome packaged into a capsid derived from capsid proteins encoded by a non-natural capsid cap gene. Non-limited examples of AAV include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), AAV type DJ (AAV-DJ), avian AAV, bovine AAV, canine AAV, equine AAV, primate AAV, non-primate AAV, and ovine AAV. In some instances, the AAV is described as a “Primate AAV,” which refers to AAV that infect primates. Likewise an AAV may infect bovine animals (e.g., “bovine AAV”, and the like). In some instances, the AAV is wild type, or naturally occurring. In some instances the AAV is recombinant.

The term “AAV capsid” as used herein refers to a capsid protein or peptide of an adeno-associated virus. In some instances, the AAV capsid protein is configured to encapsidate genetic information (e.g., a heterologous nucleic acid, a transgene, therapeutic nucleic acid, viral genome). In some instances, the AAV capsid of the instant disclosure is a variant AAV capsid, which means in some instances that it is a parental or wild-type AAV capsid that has been modified in an amino acid sequence of the parental AAV capsid protein.

The term “AAV genome” as used herein can refer to nucleic acid polynucleotide encoding genetic information related to the virus. The genome, in some instances, comprises a nucleic acid sequence flanked by AAV inverted terminal repeat (ITR) sequences. The AAV genome can be a recombinant AAV genome generated using recombinatorial genetics methods, and which can include a heterologous nucleic acid (e.g., transgene) that comprises and/or is flanked by the ITR sequences.

The term “rAAV” refers to a “recombinant AAV”. In some embodiments, a recombinant AAV has an AAV genome in which part or all of the rep and cap genes have been replaced with heterologous sequences. The term “AAV particle”, “AAV nanoparticle”, or an “AAV vector” as used interchangeably herein refers to an AAV virus or virion comprising an AAV capsid within which is packaged a heterologous DNA polynucleotide, or “genome”, comprising nucleic acid sequence flanked by AAV inverted terminal repeat (ITR) sequences. In some cases, the AAV particle is modified relative to a parental AAV particle.

The term “cap gene” refers to the nucleic acid sequences that encode capsid proteins that form, or contribute to the formation of, the capsid, or protein shell, of the virus. In the case of AAV, the capsid protein may be VP1, VP2, or VP3. For other parvoviruses, the names and numbers of the capsid proteins can differ.

The term “rep gene” refers to the nucleic acid sequences that encode the non-structural proteins (rep78, rep68, rep52 and rep40) required for the replication and production of virus.

As used herein, “native” or “wild type” can be used interchangeably, and can refer to the form of a polynucleotide, gene or polypeptide as found in nature with its own regulatory sequences, if present.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

The term “exogenous” gene as used herein is meant to encompass all genes that do not naturally occur within the genome of an individual. For example, a miRNA could be introduced exogenously by a virus, e.g. an AAV nanoparticle.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those commonly known and used in the art. Standard techniques can be used for chemical syntheses, chemical analyses, pharmaceutical preparation, formulation, and delivery, and treatment of patients.

### Self-Assembling System

Protein misfolding and aggregation may begin decades before onset of clinical symptoms and the aggregates display substantial heterogeneity across patients. Capturing the slow progression, heterogeneity and cell-type-specific vulnerability characteristic of human proteinopathies in experimental models remains a major challenge. Most existing models rely on toxicants, overexpression of disease-related proteins or exogenous seeding with fibrils. These approaches often lack temporal and cell-type control and fail to recapitulate key aspects of disease pathology. A notable example is Parkinson's disease (PD), which is characterized by the progressive loss of dopaminergic (DA) neurons and the formation of intracellular inclusions known as Lewy bodies (LBs) and Lewy neurites (LNs). The main component of LBs and LNs is misfolded α-synuclein (αSyn). It remains unclear when and where the αSyn aggregation begins, how it spreads between cells, and why the DA neurons are particularly vulnerable to degeneration. Numerous in vitro and in vivo models have been developed to model and study PD. Toxicant-based models, such as 6-hydroxydopamine (6-OHDA), induce rapid DA neuron degeneration but typically fail to produce LBs and LNs pathology. Genetic models, including overexpression of αSyn, often do not exhibit key pathological hallmarks. More recently, the αSyn pre-formed fibril (PFF) model has gained prominence, as intracerebral injection of αSyn PFFs induces neurodegeneration and behavioral deficits in rodents. However, this model requires invasive surgical procedures and lacks cell-type specificity. A system that allows precise and programmable modeling of protein aggregation in defined cell types could transform the ability to study disease mechanisms across diverse neurodegenerative conditions.

Provided herein includes a genetically-coded, modular platform that enables inducible, tunable, and cell-type-specific formation of intracellular protein aggregates across diverse in vitro and in vivo experimental models. The broad applicability of this platform is demonstrated across in vitro and in vivo experimental systems including cell lines, primary neurons, hESC or iPSC-derived neurons, organoids, and live animals. A self-assembling system, as used herein, can refer to a protein expression system (e.g., one or more expression construct or one or more expression vector), upon expression in a cell, capable of generating a proteopathic fusion protein that can self-assemble into intracellular protein aggregates and propagate the aggregation (e.g., from one cell to other cells or from one region to other regions). The self-assembling systems described herein provide a modular, programmable framework for modeling protein aggregation across a wide range of diseases.

Some of the systems, methods and compositions disclosed herein are also disclosed in Fan Y. et al. “Programmable self-assembling system to model intracellular protein aggregation and decode neurodegenerative diseases” (bioRxiv 2025.03.06.641009; doi.org/10.1101/2025.03.06.641009), the content of which is incorporated herein by reference in its entirety.

Provided herein includes a polynucleotide encoding a self-assembling proteopathic fusion protein, and related expression systems, vectors, compositions and kits comprising the polynucleotide. Upon expression in a cell, the self-assembling proteopathic fusion protein can form intracellular protein aggregates that recapitulate pathological features of protein aggregation diseases. The polynucleotides, and the related expression vectors and systems can enable inducible, tunable and cell-type-specific modeling of aggregate biology across diverse contexts across in vitro and in vivo experimental systems including cell lines, primary neurons, hESC or iPSC-derived neurons, organoids and live animals, and provide a modular, programmable framework for modeling protein aggregation across a wide range of diseases.

In some embodiments, a polynucleotide encoding a self-assembling proteopathic fusion protein comprises a first nucleic acid unit encoding a proteopathic polypeptide and a second nucleic acid unit encoding a self-assembling domain. Upon expression in a cell, the polynucleotide can be translated into a self-assembling proteopathic fusion protein comprising the proteopathic polypeptide connected to the self-assembling domain, and the self-assembling proteopathic fusion protein is capable of spontaneously forming intracellular protein aggregations in the cell. The proteopathic polypeptide may be connected to the self-assembling domain directly or indirectly via a linker. Accordingly, in some embodiments, the polynucleotide further comprises a nucleic acid unit encoding a linker.

As used herein, the term “proteopathic polypeptide” (also referred to as “aggregating disease protein”) refers to polypeptides associated with proteinopathies, also known as proteopathies, a class of diseases characterized by abnormal folding, aggregation, and/or accumulation of proteins within cells. The proteopathic polypeptides described herein refer to proteins that can misfold into abnormal, self-propagating structure (e.g., corrupting normal, identical proteins, causing a chain reaction of misfolding), leading to accumulation in tissues and causing proteinopathies. In some embodiments, the proteopathic polypeptide is from any neurodegenerative disease target protein that aggregates in neurodegenerative diseases. In some embodiments, the proteopathic polypeptide described herein can comprise a polypeptide that upon self-aggregation adopts a misfolded conformation that may be rich in β-sheets (e.g., cross-0 motif).

Exemplary proteopathic polypeptide include, but are not limited to, alpha synuclein polypeptide, Tau, TDP-43, an Aβ precursor protein, an Aβ peptide, a prion, serum amyloid A, amyloid β-protein, transthyretin, cystatin C, beta-2-microglobulin, apolipoprotein AI, apolipoprotein AII, gelsolin, amylin, calcitonin, atrial natriuretic factor, lysozyme, insulin, fibrinogen α-A chain, superoxide dismutase 1, huntingtin, an androgen receptor, atrophin, an ataxin, immunoglobulin light chain, neuroserpin, superoxide dismutase 1, and a TATA box-binding protein. In some embodiments, the proteopathic polypeptide is selected from the group consisting of TDP-43, Alpha synuclein, Tau, Fus, TIA1, SOD1, Huntingtin, Ataxin 2, hnRNPA1, hnRNPA2B1, EWS RNA Binding Protein 1, and TATA box binding protein factor 15.

In some embodiments, the proteopathic polypeptide is α-synuclein polypeptide. α-synuclein refers to α-synuclein as well as variants and/or fragments thereof (e.g., associated with alpha-synucleinopathies such as Parkinson's disease, dementia with Lewy bodies, and multiple system atrophy). In some embodiments, the α-synuclein polypeptide may comprise variants and/or fragments of α-synuclein that possess the ability to self-aggregate into Lewy-bodies or Lewy-body-like structures as defined herein.

In some embodiments, the proteopathic polypeptide is Tau protein (MAPT) or a variant or a fragment thereof. The term “tau protein” refers generally to any protein of the tau protein family. Tau proteins are characterized as being one among a larger number of protein families which co-purify with microtubules during repeated cycles of assembly and disassembly. Tau protein is encoded by the MAPT (microtubule-associated protein tau) gene and is also known as microtubule-associated-proteins (MAPs). The Tau polypeptide used herein can include different isoforms generated by alternative splicing of the MAPT gene. The isoforms differ in the number of microtubule-binding repeats and/or N-terminal insert domains. Abnormal tau aggregation leads to a class of neurodegenerative disorders also known as tauopathies, including, for example, Alzheimer's disease, frontotemporal dementia, progressive supranuclear palsy, and corticobasal degeneration.

In some embodiments, the proteopathic polypeptide used herein is a TDP-43 (TAR DNA-binding protein 43) polypeptide or a variant or a fragment thereof. TDP-43, encoded by the TARDBP gene, is a ubiquitously expressed RNA/DNA-binding protein that plays a central role in RNA metabolism. Pathological misfolding, aggregation, and cytoplasmic accumulation of TDP-43 are central drivers in several neurodegenerative disorders including amyotrophic lateral sclerosis, frontotemporal dementia.

In some embodiments, the proteopathic polypeptide used herein can comprise an amino acid sequence set forth in SEQ ID Nos: 1-13 or an amino acid sequence having at least 80%, at least 85%, at least 90%, at least 95%, at least 96%, at least 97%, at least 98%, at least 99%, or 100% sequence identity to any one of SEQ ID Nos: 1-13.

The present disclosure is described with respect to any proteopathic polypeptide or aggregating disease protein that may initiate or undergo a pathological aggregation similar to Tau, alpha-synuclein, or TDP-43 by virtue of conformational change in a domain critical for initiation and propagation of the aggregation.

In some embodiments, the proteopathic polypeptide described herein may be associated with a proteinopathy including, for example, a neurodegenerative disease, a proliferative disease, an inflammatory disease, a cardiovascular disease, diabetes, a synucleinopathy, Parkinson's disease, dementia with Lewy bodies, diffuse Lewy body disease, multiple system atrophy, a tauopathy, Alzheimer's disease, Creutzfeldt-Jakob disease or other prion disease, spinocerebellar ataxias, spinal or bulbar muscular atrophy, amyotrophic lateral sclerosis, hereditary renal amyloidosis, medullary carcinoma of the thyroid, familial amyloid polyneuropathy, amyloidosis, or frontotemporal degeneration. In some embodiments, the proteopathic polypeptide described herein is associated with a neurodegenerative disease such as Alzheimer's disease, Parkinson's disease, amyotrophic lateral sclerosis (ALS), Huntington's disease, frontotemporal dementia (FTD), multiple system atrophy (MSA) or Lewy body dementia (LBD).

The wording “associated with” as used herein with reference to two items indicates a relation between the two items such that the occurrence of a first item is accompanied by the occurrence of the second item, which includes but is not limited to a cause-effect relation and sign/symptoms-disease relation. For example, TDP-43 is associated with ALS, frontotemporal dementia (FTD), Alzheimer's disease (AD), chronic traumatic encephalopathy (CTE). Alpha synuclein is associated with Parkinson's disease (PD), Lewy body dementia (LBD), AD, and multiple system atrophy (MSA). Tau is associated with AD, FTD, CTE and PD. FUS is associated with FTD and ALS. Other proteopathic proteins such as SOD1, TIA1, ataxin 2, hnRNPA1, hnRNPA2B1, EWS RNA binding protein 1, and TATA box binding protein factor 15 are associated with ALS. A person skilled in the art would be able to identify other proteopathic polypeptides and their associated proteinopathies.

The proteopathic polypeptide described herein is linked to a self-assembling domain to form a self-assembling proteopathic fusion protein that can initiate and propagate the aggregation in a cell. As used herein, a “self-assembling domain” is a polypeptide having the ability to spontaneously organized into higher-order structures such as filaments, tubes, sheets, or scaffolds through non-covalent interactions. Exemplary self-assembling domains include, but are not limited to, cytoskeletal filaments (e.g., actin filaments, microtubules), amyloid fibrils, actin family proteins (α-actin, β-actin, and γ-actin), tubulin family proteins (α-tubulin, β-tubulin, and γ-tubulin) gamma-prefoldin, viral capsid proteins (e.g., HIV-1 capsid protein, Hepatitis B core antigen), cage proteins (e.g., ferritin), proteins driving phase separation (e.g., fused in sarcoma (FUS) protein, heterogeneous nuclear ribonucleoprotein AI (hnRNPA1)), or a variant or a fragment thereof. In some embodiments, the self-assembling domain is an engineered protein with assembly-gain-of-function mutation. Examples of engineered proteins with assembly-gain-of-function mutation include, but are not limited to, isoaspartyl dipeptidase with E239Y mutation (1POK (E239Y)), ketopantoate hydroxymethyl transferase with E158L mutation (1M3U (E158L)), acid induced arginine decarboxylase with K491L, D494L, D497L mutation (2VYC (K491L, D494L, D497L)), E. coli AsnC with K126Y, D1313Y mutation (2CG4 (K126Y, D131Y)). Methods to engineer proteins for desired functionality include, for example, directed evolution (iterative mutation and selection) and site-directed mutagenesis to create libraries of protein variants to identify those with the desired, enhanced assembly properties, as will be understood by a person skilled in the art. In some embodiments, the self-assembling domain used herein can be designed de novo using computational design models. In some embodiments, the self-assembling domain used herein can be any of the self-assembling polypeptides disclosed in US2021/0324011A1, the content of which is incorporated herein by reference in its entirety for all purposes.

In some embodiments, the self-assembling domain used herein can comprise an amino acid sequence of any one of SEQ ID Nos: 14-22 or an amino acid sequence having at least 80%, at least 85%, at least 90%, at least 95%, at least 96%, at least 97%, at least 98%, at least 99%, or 100% sequence identity to any one of SEQ ID Nos: 14-22.

In some embodiments, the self-assembling proteopathic fusion protein described herein can comprise (1) a proteopathic polypeptide having an amino acid sequence of any one of SEQ ID Nos: 1-13 or an amino acid sequence having at least 80%, at least 85%, at least 90%, at least 95%, at least 96%, at least 97%, at least 98%, at least 99%, or 100% sequence identity to any one of SEQ ID Nos: 1-13; and (2) a self-assembling domain having an amino acid sequence of any one of SEQ ID Nos: 14-22 or an amino acid sequence having at least 80%, at least 85%, at least 90%, at least 95%, at least 96%, at least 97%, at least 98%, at least 99%, or 100% sequence identity to any one of SEQ ID Nos: 14-22.

In some embodiments, a polynucleotide encoding a self-assembling proteopathic fusion protein further comprises a molecular tag. As used herein, a “molecular tag” includes any nucleic acid sequence encoding a molecule which may facilitate detection and purification of the fusion protein described herein. The molecular tag can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mScarlet, mApple, mCitrine, mCherry, mruby3, rsCherry, rsCherryRev, mScarlet, derivatives thereof, or any combination thereof. In some embodiments, the molecular tag is an epitope tag including for example V5 tag, HA tag, FLAG tag, c-Myc tag, T7 tag, and others identifiable to a person skilled in the art. In some embodiments, the molecular tag is an affinity tag, including for example His tag, GST tag, MBP tag, Strep-tag, and other affinity tag identifiable to a person skilled in the art.

Provided herein also include self-assembling proteopathic fusion proteins encoded by the polynucleotide described herein. The self-assembling proteopathic fusion protein can comprise a proteopathic polypeptide connected to a self-assembling domain directly or indirectly via a linker. The self-assembling proteopathic protein can spontaneously form protein aggregates in a cell. Upon self-aggregation in a cell, the self-assembling proteopathic fusion protein can exhibit prior-like seeding activity towards endogenous proteopathic polypeptides. As used herein, the term “seeding” refers to the ability of the self-assembling proteopathic fusion protein to nucleate (i.e., induce or trigger) self-aggregation or aggregation of endogenous proteopathic polypeptides that lack the self-assembling domain or normally lack the ability to aggregate on their own. The present disclosure has demonstrated in exemplary in vitro, in vivo or ex vivo systems including HEK cells, primary neurons, human brain organoids, and mice, self-assembling proteopathic fusion proteins such as self-assembling αSyn (SAS), Tau (SA-Tau) and TDP-43 (SA-TDP) can induce histopathology that closely mirror that in human diseases.

In some embodiments, the self-assembling proteopathic fusion protein described herein comprises an amino acid sequence selected from SEQ ID Nos: 23-52 or an amino acid sequence having at least 80%, at least 85%, at least 90%, at least 95%, at least 96%, at least 97%, at least 98%, at least 99% sequence identity to any one of SEQ ID Nos: 23-52.

Provided herein also includes an expression vector comprising the polynucleotide encoding the self-assembling proteopathic fusion protein. The expression vector can comprise one or more promoters operably connected to a polynucleotide comprising a first nucleic acid unit encoding a proteopathic polypeptide and a second nucleic acid unit encoding a self-assembling domain, wherein the expression vector, upon expression in a cell, encodes a self-assembling proteopathic fusion protein comprising the proteopathic polypeptide connected to the self-assembling domain and capable of forming intracellular protein aggregates in the cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature. The promoter used herein can be a tissue-specific promoter (e.g., neuronal cell-specific promoter).

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The promoter can vary in length, for example be less than 1 kb. In other embodiments, the promoter is greater than 1 kb. The promoter can have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 bp, or a number or a range between any two of these values, or more than 800 bp. The promoter may provide expression of a self-assembling proteopathic fusion protein for a period of time in targeted tissues such as, but not limited to, the CNS. Expression of the self-assembling proteopathic fusion protein can be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or a number or a range between any two of these values, or more than 65 years.

In some embodiments, the promoter used herein can comprise: a minimal promoter (e.g., TATA, miniCMV, and/or miniPromo); a tissue-specific promoter and/or a lineage-specific promoter; and/or a ubiquitous promoter (e.g., a minEfla promoter, a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human R-actin (HBA) promoter, chicken β-actin (CBA) promoter, a CAG promoter, a CASI promoter, a CBH promoter, or any combination thereof).

In some embodiments, the promoter and/or other regulatory control elements comprised in the vector can influence the expression of the RNA and/or protein products encoded by the polynucleotide within desired cells of the subject.

Expression control elements and promoters include those active in a particular tissue or cell type, referred to herein as a “tissue- or cell-specific expression control elements/promoters.” Tissue-specific expression control elements are typically active in specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

In some embodiments, the promoter operably linked to the polynucleotide encoding a self-assembling proteopathic fusion protein is a tissue-specific promoter. The tissue-specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a hSynapsin promoter, a α-myosin heavy chain (a-MHC) promoter, or a cardiac Troponin T (cTnT) promoter. The tissue specific promoter can be a neuronal activity-dependent promoter and/or a neuron-specific promoter (e.g., a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav 1.7 promoter, a Nav 1.8 promoter, a Nav 1.9 promoter, or an Advillin promoter). The tissue specific promoter can be a muscle-specific promoter. The muscle-specific promoter can comprise a creatine kinase (MCK) promoter.

In some embodiments, the promoter operably linked to the polynucleotide encoding a self-assembling proteopathic fusion protein is a cell-specific promoter. The cell-specific promoter can be a neuron cell specific promoter such as, for example, a tyrosine hydroxylase promoter, a melanopsin promoter, or a promoter that expresses in retinal neurons. In another embodiment, the neuron cell specific promoter can be a PRSx8 promoter which specifically targets catecholaminergic neurons. PRSx8 is based on an upstream regulatory site in the human Dopamine Beta-Hydroxylase (“DBH”) promoter and drives high levels of expression in adrenergic neurons. In another embodiment, the neuron-specific promoter can be preprotachykinin-1 promoter (TAC-1).

Expression control elements also can confer expression in a manner that is regulatable, that is, a signal or stimuli increases or decreases expression of the operably linked polynucleotide. A regulatable element that increases expression of the operably linked polynucleotide in response to a signal or stimuli is also referred to as an “inducible element” (that is, it is induced by a signal). Particular examples include, but are not limited to, a hormone (for example, steroid) inducible promoter or an antibiotics inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

In some embodiments, the promoter operably linked to the polynucleotide encoding a self-assembling proteopathic fusion protein is an inducible promoter. An “inducible promoter” is one that is characterized by initiating or enhancing transcriptional activity when in the presence of, influenced by, or contacted by an inducing agent. An inducing agent may be endogenous or a normally exogenous condition, compound, agent, or protein that contacts a nucleic acid in such a way as to be active in inducing transcriptional activity from the inducible promoter. In certain embodiments, an inducing agent is a tetracycline-sensitive protein (e.g., tTA or rtTA, TetR family regulators). Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (TetR, or TetR-KRAB), a tetracycline operator sequence (tetO) and a tetracycline transactivator fusion protein (tTA), and a tetracycline operator sequence (tetO) and a reverse tetracycline transactivator fusion protein (rtTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), pH-regulated promoters, and light-regulated promoters. Additional non-limiting examples of inducible promoters include mifepristone-responsive promoters (e.g., GAL4-Elb promoter) and coumermycin-responsive promoters.

In some embodiments, a tissue- or cell-specific promoter can be operably linked to a transactivator polynucleotide comprising a transactivator gene. A transactivator is a protein, often a transcription factor, that binds to a specific DNA sequence to enhance, turn on, or regulate gene expression. For example, the tissue or cell-specific promoter can be a neuron-specific promoter (e.g., human synapsin promoter) capable of inducing transcription of the transactivator gene to generate a transactivator transcript which can be translated to generate a transactivator. Therefore, the activity of the cell/tissue-specific promoter and/or the degree of expression of the transactivator can be associated with the presence and/or amount a unique cell type.

In some embodiments, a tissue- or cell-specific promoter, a transactivator and a transactivator-binding compound can be used together with an inducible promoter operably connected to a polynucleotide described herein to generate a self-assembling proteopathic fusion protein. The primary promoter operably connected to the polynucleotide encoding a self-assembling domain can be an inducible promoter comprising one or more copies of a transactivator recognition sequence that the transactivator is capable of binding to induce transcription, and the transactivator can be incapable of binding the transactivator recognition sequence in the absence of the transactivator-binding compound. For example, the primary promoter operably connected to the polynucleotide encoding a self-assembling proteopathic fusion protein can comprise a tetracycline response element (TRE), and the TRE can comprise one or more copies of a tet operator (TetO). The transactivator can comprise a reverse tetracycline-controlled transactivator (rtTA). A “reverse tetracycline transactivator” (“rtTA”), as used herein, is an inducing agent that binds to a TRE promoter (e.g., a TRE3G, a TRE2 promoter, or a P tight promoter) in the presence of tetracycline (e.g., doxycycline) and is capable of driving expression of a transgene that is operably linked to the TRE promoter. rtTAs generally comprise a mutant tetracycline repressor DNA binding protein (TetR) and a transactivation domain. The mutant TetR domain is capable of binding to a TRE promoter when bound to tetracycline. The transactivator can comprise a tetracycline-controlled transactivator (tTA) for a Ted-Off system. The transactivator-binding compound can comprise tetracycline, doxycycline or a derivative thereof.

Accordingly, in some embodiment, the expression vector comprises (1) a first promoter operably connected to a first polynucleotide comprising a first nucleic acid unit encoding a proteopathic polypeptide and a second nucleic acid unit encoding a self-assembling domain; and (2) a second promoter operably connected to a second polynucleotide encoding a transactivator, wherein the first promoter is an inducible promoter capable of inducing transcription of the first polynucleotide comprising the first nucleic acid unit and the second nucleic acid unit in the presence of the transactivator and a transactivator-binding compound, wherein the first polynucleotide, upon expression in a cell, encodes a self-assembling proteopathic fusion protein comprising the proteopathic polypeptide connected to the self-assembling domain and capable of forming intracellular protein aggregates in the cell. In some embodiments, the second promoter is a cell-specific or tissue-specific promoter.

In some embodiments, the first promoter operably connected to a first polynucleotide is in a vector different from the second promoter operably connected to a second polynucleotide. Accordingly, provided herein also includes an expression system for enabling inducible, tunable, and cell-type-specific formation of intracellular protein aggregates. In some embodiments, the expression system comprises a first expression vector comprising a first promoter operably connected to a first polynucleotide comprising a first nucleic acid unit encoding a proteopathic polypeptide and a second nucleic acid unit encoding a self-assembling domain, and a second expression vector comprising a second promoter operably connected to a second polynucleotide encoding a transactivator, wherein the first promoter is an inducible promoter capable of inducing transcription of the first polynucleotide in the presence of the transactivator and a transactivator-binding compound and the second promoter is a cell-specific or tissue-specific promoter, wherein the first polynucleotide, upon expression in a cell, encodes a self-assembling proteopathic fusion protein comprising the proteopathic polypeptide connected to the self-assembling domain and capable of forming intracellular protein aggregates in the cell.

In some embodiments, the first inducible promoter comprises one or more copies of a transactivator recognition sequence the transactivator is capable of binding to induce transcription, wherein the transactivator is incapable of binding the transactivator recognition sequence in the absence of the transactivator-binding compound. In some embodiments, the first inducible promoter is a TRE. In some embodiments, the one or more copies of a transactivator recognition sequence comprise one or more copies of a tet operator (TetO). In some embodiments, the transactivator encoded by the second polynucleotide comprises rtTA, and the transactivator-binding compound comprises tetracycline, doxycycline, or a derivative thereof. In some embodiments, the second promoter is a neuron-specific promoter, including for example hSyn/Syn1, CaMKIIa, NSE/ENO2, MAP2, NF-L, NF-M, NF-H, Thy1, and others identifiable to a person skilled in the art.

Provided herein also includes a cell comprising an expression vector as described herein. In some embodiments, the cell may be a neuronal cell, a brain cell, an induced pluripotent stem cell, a mammalian cell, a neuroblastoma, an immune cell, or a blood cell.

Provided herein also includes a transgenic animal model comprising an expression vector, an expression system, or a cell as described herein. In some embodiments, the animal may be a mouse, rat or other rodent, a non-human primate, fish, nematode, yeast, or a fly.

### Viral Delivery of the Self-Assembling Expression System

The polynucleotide encoding a self-assembling proteopathic fusion protein and related expression vectors and systems can be comprised in a plasmid or in a virus or viral vector.

A viral vector can be, can comprise, or can be derived from, an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector derivatives thereof, or any combination thereof.

In some embodiments, the viral vector is a recombinant lentiviral vector. In some embodiments, the viral particle is a lentiviral particle. Lentiviruses are positive-sense, ssRNA retroviruses with a genome of approximately 10 kb. Lentiviruses are known to integrate into the genome of dividing and non-dividing cells. Lentiviral particles may be produced, for example, by transfecting multiple plasmids (typically the lentiviral genome and the genes required for replication and/or packaging are separated to prevent viral replication) into a packaging cell line, which packages the modified lentiviral genome into lentiviral particles. In some embodiments, a lentiviral particle may refer to a first generation vector that lacks the envelope protein. In some embodiments, a lentiviral particle may refer to a second generation vector that lacks all genes except the gag/pol and tat/rev regions. In some embodiments, a lentiviral particle may refer to a third generation vector that only contains the endogenous rev, gag, and pol genes and has a chimeric LTR for transduction without the tat gene (see Dull. T. et al. (1998) J. Virol. 72:8463-71). For further description, see e.g., Durand, S. and Cimarelli, A. (2011) Viruses 3:132-59.

Use of any lentiviral vector is considered within the scope of the disclosed systems, compositions and methods. In some embodiments, the lentiviral vector is derived from a lentivirus including, without limitation, human immunodeficiency virus-1 (HIV-1), human immunodeficiency virus-2 (HIV-2), simian immunodeficiency virus (SIV), feline immunodeficiency virus (FIV), equine infectious anemia virus (EIAV), bovine immunodeficiency virus (BIV), Jembrana disease virus (JDV), visna virus (VV), and caprine arthritis encephalitis virus (CAEV).

In some embodiments, the viral vector is encapsidated in a viral particle. In some embodiments, the viral particle is a recombinant lentiviral particle encapsidating a recombinant lentiviral vector. In some embodiments, the recombinant viral particles comprise a lentivirus vector in combination with one or more foreign viral capsid proteins. Such combinations may be referred to as pseudotyped recombinant lentiviral particles. In some embodiments, foreign viral capsid proteins used in pseudotyped recombinant lentiviral particles are derived from a foreign virus. In some embodiments, the foreign viral capsid protein used in pseudotyped recombinant lentiviral particles is Vesicular stomatitis virus glycoprotein (VSV-GP). VSV-GP interacts with a ubiquitous cell receptor, providing broad tissue tropism to pseudotyped recombinant lentiviral particles. In addition, VSV-GP is thought to provide higher stability to pseudotyped recombinant lentiviral particles. In other embodiments, the foreign viral capsid proteins are derived from, including without limitation. Chandipura virus. Rabies virus. Mokola virus, Lymphocytic choriomeningitis virus (LCMV). Ross River virus (RRV), Sindbis virus, Semliki Forest virus (SFV), Venezuelan equine encephalitis virus. Ebola virus Reston, Ebola virus Zaire, Marburg virus, Lassa virus, Avian leukosis virus (ALV), Jaagsiekte sheep retrovirus (JSRV). Moloney Murine leukemia virus (MLV). Gibbon ape leukemia virus (GALV). Feline endogenous retrovirus (RD114). Human T-lymphotropic virus 1 (HTLV-1), Human foamy virus, Maedi-visna virus (MVV), SARS-CoV, Sendai virus, Respiratory syncytia virus (RSV), Human parainfluenza virus type 3, Hepatitis C virus (HCV), Influenza virus. Fowl plague virus (FPV), or Autographa californica multiple nucleopolyhedro virus (AcMNPV).

In some embodiments, the recombinant lentiviral vector is derived from a lentivirus pseudotyped with vesicular stomatitis virus (VSV), lymphocytic choriomeningitis virus (LCMV), Ross river virus (RRV), Ebola virus. Marburg virus, Mokala virus, Rabies virus, RD114, or variants therein. Examples of vector and capsid protein combinations used in pseudotyped Lentivirus particles can be found, for example, in Cronin. J. et al. (2005). Curr. Gene Ther. 5(4):387-398. Different pseudotyped recombinant lentiviral particles can be used to optimize transduction of particular target cells or to target specific cell types within a particular target tissue (e.g., a diseased tissue). For example, tissues targeted by specific pseudotyped recombinant lentiviral particles, include without limitation, liver (e.g. pseudotyped with a VSV-G, LCMV, RRV, or SeV F protein), lung (e.g., pseudotyped with an Ebola, Marburg, SeV F and HN, or JSRV protein), pancreatic islet cells (e.g., pseudotyped with an LCMV protein), central nervous system (e.g., pseudotyped with a VSV-G. LCMV. Rabies, or Mokola protein), retina (e.g, pseudotyped with a VSV-G or Mokola protein), monocytes or muscle (e.g., pseudotyped with a Mokola or Ebola protein), hematopoietic system (e.g., pseudotyped with an RD114 or GALV protein), or cancer cells (e.g., pseudotyped with a GALV or LCMV protein). For further description, see Cronin, J. et al. (2005). Curr. Gene Ther. 5(4):387-398 and Kay, M. et al. (2001) Nat. Med. 7(1):33-40. In some embodiments, the recombinant lentiviral particle comprises a capsid pseudotyped with vesicular stomatitis virus (VSV), lymphocytic choriomeningitis virus (LCMV). Ross river virus (RRV). Ebola virus, Marburg virus. Mokala virus, Rabies virus. RD114 or variants therein.

A viral vector can be a lentiviral vector (e.g., human immunodeficiency virus 1 (HIV-1), human immunodeficiency virus 2 (HIV-2), visna-maedi virus (VMV) virus, caprine arthritis-encephalitis virus (CAEV), equine infectious anemia virus (EIAV), feline immunodeficiency virus (FIV), bovine immune deficiency virus (BIV), simian immunodeficiency virus (SIV), derivatives thereof, or any combination thereof). A viral vector can be a recombinant lentiviral vector. The recombinant lentiviral vector can be derived from a lentivirus pseudotyped with vesicular stomatitis virus (VSV), lymphocytic choriomeningitis virus (LCMV), Ross river virus (RRV), Ebola virus, Marburg virus, Mokala virus, Rabies virus, RD114, or variants therein. The viral vector composition can comprise: one or more of a left (5′) retroviral LTR, a Psi (Ψ) packaging signal, a central polypurine tract/DNA flap (cPPT/FLAP), a retroviral export element, and a right (3′) retroviral LTR. The promoter of the 5′ LTR can be replaced with a heterologous promoter. The 5′ LTR or 3′ LTR can be a lentivirus LTR. The 3′ LTR can comprise one or more modifications and/or deletions. The 3′ LTR can be a self-inactivating (SIN) LTR.

In some embodiments, the viral vector is an adeno-associated virus (AAV) vector. Adeno-associated virus (AAV) is a replication-deficient parvovirus, the single-stranded DNA genome of which is about 4.7 kb in length including 145 nucleotide inverted terminal repeat (ITRs). The ITRs play a role in integration of the AAV DNA into the host cell genome. When AAV infects a host cell, the viral genome integrates into the host's chromosome resulting in latent infection of the cell. In a natural system, a helper virus (for example, adenovirus or herpesvirus) provides genes that allow for production of AAV virus in the infected cell. In the case of adenovirus, genes E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating.

AAV vectors that comprise coding regions of one or more proteins of interest (e.g., the self-assembling proteopathic fusion protein) are provided. The AAV vector can include a 5′ inverted terminal repeat (ITR) of AAV, a 3′ AAV ITR, a promoter, and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the AAV vector includes a posttranscriptional regulatory element downstream of the restriction site and upstream of the 3′ AAV ITR. In some embodiments, the AAV vectors disclosed herein can be used as AAV transfer vectors carrying a transgene encoding a protein of interest for producing recombinant AAV viruses that can express the protein of interest in a host cell.

The AAV vector can comprise single-stranded AAV (ssAAV) vector or a self-complementary AAV (scAAV) vector. The AAV vector can comprise AAV1, AAV2, AAV3, AAV4, AAV5, AAV6, AAV7, AAV8, AAV9, derivatives thereof, or any combination thereof. The AAV vector can comprise an AAV9 variant engineered for systemic delivery (e.g., AAV-PHP.B, AAV-PHP.eB, or AAV-PHP.S). The AAV vector can be or can comprise an AAV selected from the group consisting of AAV9, AAV9 K449R (or K449R AAV9), AAV1, AAVrhlO, AAV-DJ, AAV-DJ8, AAV5, AAVPHP.B (PHP.B), AAVPHP.A (PUPA), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2 A15/G2AB (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3. 1/hu.6, AAV3.1/hu.9, AAV3-9/rh.52, AAV3-11/rh.53, AAV4-8/rl1.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106. 1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LKO1, AAV-LK02, AAV-LK03, AAV-LK04, AAV-LK05, AAV-LK06, AAV-LK07, AAV-LK08, AAV-LKO9, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128. 1/hu.43, true type AAV (ttAAV), EGRENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1 AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6. 1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1 AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-1, AAV CLv13, AAV CLv-13AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1 AAV CLv-R2, AAV CLv-R3, AAV CLv-R4, AAV CLv-R5, AAV CLv-R6, AAV CLv-R7, AAV CLv-R8, AAV CLv-R9, AAV CSp-1 AAV CSp-10AAV CSp-11AAV CSp-2, AAV CSp-3, AAV CSp-4, AAV CSp-6, AAV CSp-7, AAV CSp-8, AAV CSp-8. 10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, variants thereof, a hybrid or chimera of any of the foregoing AAV serotypes, or any combination thereof.

The AAV can be an AAV derived from a naturally occurring “wild-type” virus, an AAV derived from a rAAV genome packaged into a capsid derived from capsid proteins encoded by a naturally occurring cap gene and/or a rAAV genome packaged into a capsid derived from capsid proteins encoded by a non-natural capsid cap gene.

In some embodiments, the viral vector is an rAAV. In some embodiments, a recombinant AAV has an AAV genome in which part or all of the rep and cap genes have been replaced with heterologous sequences. The genome of an rAAV can, for example, comprise at least one inverted terminal repeat configured to allow packaging into a vector and a cap gene. In some embodiments, it can further include a sequence within a rep gene required for expression and splicing of the cap gene. In some embodiments, the genome can further include a sequence capable of expressing VP3. In some embodiments, the only protein that is expressed is VP3 (the smallest of the capsid structural proteins that makes up most of the assembled capsid—the assembled capsid is composed of 60 units of VP proteins, ~50 of which are VP3). In some embodiments, VP3 expression alone is adequate to allow the method of screening to be adequate.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

In some embodiments, the AAV vector disclosed herein comprises a targeting peptide capable of directing the AAV to target environment (e.g., CNS, PNS, and the heart) in a subject. The systems, methods, compositions, and kits provided herein can, in some embodiments, be employed in concert with the systems, methods, compositions, and kits comprising recombinant adeno-associated virus (rAAV) comprising an AAV targeting peptide to target specific environments such as the nervous system described in US20230295659A1 (TARGETING PEPTIDES FOR DIRECTING ADENO-ASSOCIATED VIRUSES (AAVs)), the content of which is incorporated herein by reference in its entirety.

In some embodiments, the targeting peptide is capable of directing AAV to, or primarily to, the CNS of the subject (referred to as a “CNS targeting peptide”). In some embodiments, the targeting peptide is capable directing AAV to, or primarily to, the PNS of the subject (referred herein as a “PNS targeting peptide”). The CNS targeting peptide can, in some embodiments, direct AAV to deliver nucleic acids to neurons, glia, endothelia cells, astrocytes, cerebellar Purkinje cells, or a combination thereof, of the CNS. In some embodiments, the targeting peptide is capable of directing AAV to deliver nucleic acid to the CNS, the heart (e.g., cardiomyocytes in the heart), peripheral nerves, or a combination thereof, in the subject.

The AAV can, in some embodiments, comprise a targeting peptide comprising at least 4 contiguous amino acids of any of SEQ ID NOs: 1-44, 48-53 and 65-68 of U.S. application Ser. No. 18/047,233. In some embodiments, the targeting peptide sequence comprises at least 4 contiguous amino acids of QAVRTSL (SEQ ID NO: 37 of U.S. application Ser. No. 18/047,233).

In some embodiments, the targeting peptide can be inserted into any desired section of a protein of the AAV. In some embodiments, the targeting peptide can be inserted into a capsid protein. In some embodiments, the targeting peptide is inserted on a surface of the desired protein. In some embodiments, the targeting peptide is inserted into the primary sequence of the protein. In some embodiments, the targeting peptide is linked (e.g., covalently) to the protein. In some embodiments, the targeting peptide is inserted into an unstructured loop of the desired protein. In some embodiments, the unstructured loop can be one identified via a structural model of the protein.

In some embodiments, the targeting peptide is part of an AAV capsid protein. The targeting peptide sequence can be inserted between AA588-589 or between AA586-592 of an AAV sequence of the vector (SEQ ID NO: 45 of U.S. application Ser. No. 18/047,233). In some embodiments, the sequence QAVRTSL (SEQ ID NO: 37) further comprises at least two of amino acids 587, 588, 589, and 590 of SEQ ID NO: 45 of U.S. application Ser. No. 18/047,233. In some embodiments, the rAAV used herein is referred to as “AAV-PHP.eB” which is an AAV variant having a non-natural capsid protein comprising a 11-mer amino acid sequence DGTLAVPFKAQ (SEQ ID NO: 4 of U.S. application Ser. No. 18/047,233).

The targeting peptides, in some embodiments, can increase transduction efficiency of rAAV to a target environment (e.g., the CNS, the PNS, or a combination thereof) in the subject as compared to an AAV that does not contain the targeting peptides. For example, the inclusion of one or more of the targeting peptides disclosed herein in a rAAV can result in an increase in transduction efficiency by, or by at least, 10%, 20%, 30%, 40%, 50%, 60%, 70%, 80%, 90%, 100%, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 10-fold, 20-fold, 30-fold, 40-fold, 50-fold, 60-fold, 70-fold, 80-fold, 90-fold, 100-fold, or a range between any two of these values, as compared to an AAV that does not comprise the targeting peptide. In some embodiments, the increase is at least 2-fold. In some embodiments, the increase is a 40-90 fold increase. In some embodiments, the transduction efficiency is increased for transducing rAAV to the CNS. In some embodiments, the transduction efficiency is increased for transducing rAAV to the PNS. In some embodiments, the transduction efficiency is increased for transducing rAAV to cardiomyocytes, sensory neurons, dorsal root ganglia, visceral organs, or any combination thereof.

### Use of the Self-Assembling Systems

The self-assembling systems and vectors described herein can be effectively delivered to a cell. The cell can be in vitro, in vivo (in a subject) or ex vivo. In some embodiments, the self-assembling systems and vectors described herein can be delivered to a target environment (e.g., nervous system). In some embodiments, the cell is a cell which can be affected by a neurodegenerative disease. For example, the cell can be a glial cell or a neuronal cell. In some embodiment, the cell used herein is from a neuronal cell line, e.g., a neuroblastoma cell line, a primary neuron, embryonic stem cell (e.g., hESC) or iPSC-derived neurons or organoids. In one embodiment, the cell is a mammalian cell. In one embodiment, the cell is a human cell. In one embodiment, the cell is selected from the group consisting of yeast, insect, avian, fish, worm, amphibian, Xenopus, bacteria, algae and mammalian cells. In some embodiments, the subject can be an animal such as a mouse, rat or other rodent, or a non-human primate, or an animal model.

Provided herein includes a method of modeling intracellular aggregation of a proteopathic polypeptide in a cell or in a subject (e.g., a non-human animal). The method can comprise introducing into a cell an expression vector or an expression system described herein, allowing formation of intracellular protein aggregates comprising the self-assembling proteopathic fusion protein, and detecting the intracellular protein aggregates (e.g., detecting the molecular tag in the self-assembling proteopathic fusion protein).

As used herein, the term “introducing,” or “introduce,” as it relates to introducing an expression vector into a cell, refers to any method suitable for transferring the expression vector into the cell. The term includes, but is not limited to, conjugation, transformation/transfection (e.g., divalent cation exposure, heat shock, electroporation), nuclear microinjection, incubation with calcium phosphate polynucleotide precipitate, high velocity bombardment with polynucleotide-coated microprojectiles (e.g., via gene gun), lipofection, cationic polymer complexation (e.g., DEAE-dextran, polyethylenimine), dendrimer complexation, mechanical deformation of cell membranes (e.g., cell-squeezing), sonoporation, optical transfection, impalefection, hydrodynamic polynucleotide delivery, Agrobacterium-mediated transformation, transduction (e.g., transduction with a virus or viral vector), natural or artificial competence, protoplast fusion, magnetofection, nucleofection, or combinations thereof. An introduced expression vector, or a polynucleotide therefrom, can be genetically integrated or exist extrachromosomally.

Detecting intracellular protein aggregates can comprise determining the number and/or size and/or location of the intracellular protein aggregates, inclusions (e.g., tau/alpha-synuclein inclusions, cytoplasmic TDP-43 inclusions), plaques (e.g., amyloid-beta plaques), protein phosphorylation (e.g., phosphorylated tau/alpha synuclein), inclusion counts per region (e.g., per cell) and other features identifiable to a person skilled in the art.

Provided herein also includes a method of inducing a proteinopathy (e.g., a neurodegenerative disease) associated to a proteopathic polypeptide in a cell or a subject. The method can comprise introducing into the cell or the subject expression vector or an expression system described herein, expressing the self-assembling proteopathic fusion protein capable of forming intracellular protein aggregates in the cell, thereby inducing the proteinopathy associated to the proteopathic polypeptide. In some embodiments, the proteopathic polypeptide of the self-assembling proteopathic fusion protein does not include a mutation which differs from the wild-type sequence and which is known or suspected to cause or be associated with inducing a neurodegenerative disease pathology in a cell.

The method can further comprise detecting pathological features of the proteopathy in the cell or the subject. The pathological features of a proteopathy can comprise histological and transcriptional features of the disease, such as the number and/or size and/or location of the intracellular protein aggregates, tau/alpha-synuclein inclusions, amyloid-beta plaques, protein phosphorylation (e.g., phosphorylated tau/alpha synuclein), cytoplasmic TDP-43 inclusions, inclusion counts per region (e.g., per cell), increased expression of ubiquitin, and cell degeneration. Pathological features also include detection of differentially expressed genes and/or activation/deactivation of related pathways (e.g., apoptosis, inflammation, and mitochondrial dysfunction pathways). The pathological features of the proteopathy can also include behavior features which can be detected by conducting behavioral tests (e.g., open field test, forced swimming test, elevated plus-maze, beaming crossing test, cylinder test) in animals administered with the expression vector described herein.

Provided herein also includes a method of identifying a candidate compound for the treatment of a proteinopathy, such as a compound capable of modulating or inhibiting a neurodegenerative disease such as Alzheimer's disease, Parkinson's disease, ALS, Huntington's disease or FTLD. The method can be used to screen compounds that demonstrate ability to modulate or inhibit pathological protein aggregation without interference with the normal functionality of the proteopathic protein.

The method can comprise introducing into a cell or a subject an expression vector or an expression system described herein, expressing the self-assembling proteopathic fusion protein capable of forming intracellular protein aggregates in the cell, wherein the proteopathic polypeptide is associated with a proteinopathy, administering to the cell with a compound of interest, and determining the effect of the compound of interest on the proteinopathy.

In some embodiments, determining the effect of the compound of interest on the proteinopathy comprises detecting pathological features of the proteinopathy in the cell or subject, including inclusion localization, morphology and composition, and/or transcriptional responses. In some embodiments, determining the effect of the compound of interest on the proteinopathy comprises detecting behavioral phenotypes of the subject. In some embodiments, detecting the effect of the compound of interest on the proteinopathy comprises detecting the composition, number, size, morphology, and/or location of the intracellular protein aggregates. In some embodiments, the method can further comprise selecting the compound of interest as a candidate compound if a decrease in the number and/or size of intracellular protein aggregates or an improvement in the pathological features, phenotype, symptoms, and/or severity of the proteinopathy is observed when compared to a reference or a control, e.g., the comparable features obtained in the absence of the compound of interest.

### EXAMPLES

Some aspects of the embodiments discussed above are disclosed in further detail in the following examples, which are not in any way intended to limit the scope of the present disclosure.

### Example 1

### General Experimental Methods

This example describes general experimental methods and data analysis approaches used in Example 2 below.

### Animals

Animal husbandry and experimental procedures involving mice were done in accordance with protocols approved by the California Institute of Technology Institutional Animal Care and Use Committee (IACUC, protocol 1650) and the mice were handled in accordance with the principles and procedures of the Guide for the Care and Use of Laboratory Animals of the National Institutes of Health. Mice were group housed at 2-3 mice per cage with 13/11 light/dark cycles at ambient temperatures of 71-75° F. and 30-70% humidity. C57BL/6J (Jackson Labs #026267), Snca^{tm1.1Kluk/J} (Jackson Labs #035412) and heterozygous B6.Cg-7630403G23Rik^{Tg(Th-cre)1Tmd/J}(Jackson Labs #008601) mice were used in experiments. Genotyping was performed by Transnetyx. AAVs were administered via retro-orbital injection during isoflurane anesthesia (1-3% in 95% O2/5% CO2, provided by nose cone at 1 L/min), at 1×10¹² viral genomes (v.g.) per animal. A detailed protocol for systemic AAV administration through retro-orbital injection is available on protocols.io (dx.doi.org/10.17504/protocols.io.36wggnw73gk5/v1). Mice were randomly assigned to different conditions.

### Cell Lines

HEK 293 cells (ATCC, CRL-3216) were used in SAS screening and characterization. The human embryonic stem cell line (hESC) line CSES07 (Cedars Sinai, CVCL_B818) was used to derive both monolayer neurons and organoids. CSES07 was obtained from Cedars Sinai Medical Center with a verified normal karyotype and was contamination-free. All cell lines were authorized for use under the supervision of the California Institute of Technology Institutional Biosafety Committee (IBC #22-182).

### SAS Library, SA-Tau and SA-TDP Design

To investigate whether intracellular αSyn aggregation can recapitulate Parkinsonian pathology, a library of self-assembling synuclein (SAS) constructs was designed. Each construct consisted of wild-type (WT) αSyn (UniProt ID: P37840-1) fused to selected self-assembling proteins (SAPs). SAPs were chosen based on structural evidence indicating terminal accessibility and the nature of their assemblies, as revealed by structural studies. 1POK(E239Y), DHF40, and DHF46 were selected based on known filament formation (PDB entries 5LP3 and 6E9R), while 2VYC (K491L, D494L, D497L) and gamma-prefoldin (γPFD) were selected for their punctate assembly characteristics (PDB entry 6VY1). Additionally, cytoskeletal proteins 0-tubulin (PDB: 3J2U) and 3-actin (PDB: 6DJO) were included to evaluate intracellular fiber-mediated effects. Several constructs also integrated mitochondrial targeting proteins (TOMM5, TOMM20) to explore how spatial localization influences aggregation outcomes. All fusion junctions incorporated flexible glycine-rich linkers (GGSGGTGG) (SEQ ID NO: 53) and V5 epitope tags (GKPIPNPLLGLDST; SEQ ID NO: 54) for enhanced detection and characterization. SA-Tau and SA-TDP construct consisted of human Tau (isoform 2N4R) or TDP-43 fused with 1POK(E239Y). Detailed information on αSyn, Tau, TDP43, SAS, SA-Tau and SA-TDP constructs is listed in Tables 1-5.

### SAS Screening in HEK Cells

SAS plasmids were transfected into HEK cells using Lipofectamine 3000 (ThermoFisher, L3000008) following the manufacturer's instructions. Briefly, 100K HEK cells were plated in 24-well plates. The next day, 0.4 g SAS plasmids and 0.1 g rtTA plasmids were transfected into each well. Cells were incubated with lipofectamine overnight. The medium was switched to 5% FBS in DMEM/F12 the next morning, and 10 g/mL of doxycycline (Sigma, Cat #D9891-10G) was added into the medium. After 3 days of doxycycline treatment, cells were fixed and stained. For protocol, see dx.doi.org/10.17504/protocols.io.6qpvr97wzvmk/v1.

### Immunostaining of Cultured Cells

At predetermined time points, cells were removed from the incubator, washed once with phosphate-buffered saline (PBS), and fixed with 4% paraformaldehyde for 10 minutes at room temperature (RT). After washing 3 times with PBS, the blocking buffer containing 1×PBS, 0.03% Triton X-100 (PBST), and 5% normal donkey serum was applied and incubated at RT for 60 minutes. Primary antibodies were diluted in fresh blocking buffer, added on the cell, and incubated overnight at 4° C. The next day, the antibody solution was washed off with PBST for 10 minutes, 3 times. Secondary antibodies were applied for 60 minutes and then washed off with PBST for 10 minutes, 3 times. After the final wash, cells remain in PBST for imaging. Antibodies and reagents used were listed in the key resource table. For protocol, see dx.doi.org/10.17504/protocols.io.4r3129eb4vly/v1

### Western Blotting

Western blotting was performed using the iBind Automated Western System following the manufacturer's instructions. Briefly, cells were collected with cold DPBS and lysed with RIPA buffer. Total protein concentration was measured using Qubit Protein BR Assay kit and Qubit4. An equal amount of protein was run in Bio-Rad 4-20% TGX stain-free gels and then transferred to a PVDF membrane. Membranes were fixed with 4% paraformaldehyde for 10 minutes before blotting. Protein immunodetection was performed with indicated antibodies using the iBind Automated Western System. Signals were detected with Max Western ECL and imaged with a ChemiDoc imaging system. For protocol, antibody information and reagents used see dx.doi.org/10.17504/protocols.io.j8nk96p6v5r/v2.

### Viral Vector Production

AAV packaging and purification were carried out as previously described. The viruses for in vitro experiments were produced by suspension HEK cells. In brief, recombinant AAV was produced by triple transfection of cells in suspension using the VirusGEN AAV Kit with RevIT enhancer (Mirus Bio, MIR 8007) at a molar ratio of transgene:AAV capsid:pHelper=1:2:0.5, as per the manufacturer's instructions. The total DNA amount was 2 μg per mL of cells. Virus-producing cells and medium were harvested 72 hours post-transfection. The viruses used in animal experiments were produced by adherent HEK cells. In brief, HEK cells are transiently transfected using polyethyleneimine (PEI) at a molar ratio of transgene:AAV capsid:pHelper=1:4:2. 120 hours after transfection, the producer cells were harvested, and cell culture media was collected at 72- and 120-hours post-transfection. Following cell lysis and nuclease digestion, viruses from suspension cells and adherent cells were purified using iodixanol step gradient columns followed by ultracentrifugation, as previously detailed. The purified AAVs were quantified through droplet digital PCR (ddPCR) following Addgene's protocol with modifications. Briefly, AAV samples were first serially diluted to achieve a suitable concentration for ddPCR analysis. The PCR reaction mixture was prepared using ddPCR EvaGreen Supermix (Bio-Rad), primers for WPRE or ITRs, the diluted AAV sample, and nuclease-free water. The mixture was then partitioned into droplets using the QX200 Droplet Generator, and the droplets were transferred to a 96-well plate for PCR in a thermocycler. After cycling, the droplets were read using a QX200 Droplet Reader, and the concentration of the viral genome was analyzed using QuantaSoft software. The viral genome titer in original sample was then back calculated using the dilution factors. The final viral preparation was stored in DPBS with Pluronic F-68 to prevent adhesion loss. For protocol and reagents used, see dx.doi.org/10.17504/protocols.io.ewovldpeovr2/v1.

### Primary Neuron Preparation

The day before the procedure, 10×HBSS (Thermo Fisher, Cat #14065056) was diluted to 1× using sterile distilled water (Invitrogen, Cat #10977015) and pre-cooled to 4° C. Glass inserts (Neuvitro Corporation, Cat #GG-12-15 Pre) were placed in 24-well plates and were coated with Poly-D-lysine (Millipore Sigma, Cat #A-003-E) overnight. Plating media was prepared using the BrainPhys Neuronal Medium SM1 Kit (Stem Cell Technologies, Cat #05792), 1× Glutamax, (Thermo Fisher, Cat #35050061), 1× Penicillin-Streptomycin (Thermo Fisher, Cat #15140122,), and 1×1-glutamine (Bio-Techne, Cat #0218). Pregnant mice from JAX were euthanized on day 17 of pregnancy. Embryos were removed, decapitated, and brains were isolated in cold HBSS. Cortices and hippocampi were dissected, and hippocampi were quartered and placed into 15 mL tubes. Papain (Sigma Aldrich, Cat #P3125-250MG) was added for digestion, followed by washing with a stopping medium containing HBSS 1× and 10% Fetal Bovine Serum. Hippocampi were dissociated by gentle pipetting, and supernatants with dissociated neurons were collected. After centrifugation, cells were resuspended in plating media, counted, and seeded at 200,000 cells/well on Poly-D-lysine-coated inserts. After 72 hours, the media was replaced with a combination of 500 mL Neurobasal Medium (Thermo Fisher, Cat #21103049), 2 mL B27 Supplement (Thermo Fisher, Cat #17504044), and 0.5 mM L-glutamine (Bio-Techne, Cat #0218). Virus was then added for indicated times and doses. For protocol, see dx.doi.org/10.17504/protocols.io.5qpvo98ozv4o/v1.

### Continuous Neurons and Organoid Differentiation

hESCs were plated on 6-well plates coated with Vitronectin (Gibco, Cat #A14700) at 1:100 diluted in DPBS (Gibco, Cat #14190250) and maintained in E8 medium (Thermo Fisher, Cat #A1517001). The E8 medium was changed every day, and the cells were passaged every 3-5 days at 70-85% confluence. The cells were passaged using EDTA dissociation as described previously. To induce differentiation, hESCs were dissociated at 70%-80% confluence using EDTA dissociation buffer and replated as a single cell suspension on Matrigel-coated 24-well plates at a density of 100K cells/cm² in E6 medium (Thermo Fisher, Cat #A1516401) containing 10 μM ROCK-inhibitor (Y-27632; R&D, Cat #1254). The cells were kept in E6 medium+Y-27632 overnight. The next day, the medium was switched to a neuroectoderm-inducing medium. The cells were kept in E6 medium containing 10 μM SB431542 (R&D, Cat #1614), 100 nM LDN139189 (R&D, Cat #6053), and 500 nM XAV (R&D, 3748) for the first 4 days and then switched to E6 medium containing 10 μM SB and 100 nM LDN for 6 days. Then the cells were switched to progenitor expanding medium, containing neurobasal (NB) medium supplemented with 1-glutamine (Gibco, Cat #25030-164), N2 (Stem Cell Technologies, 07156), B27 (Life Technologies, Cat #17504044) and NEAA (Sigma, Cat #M7145) for 10 days. At D20, the neuron progenitors were dissociated into single cells and replated in a V-bottom ultra-low attachment 96-well plate at 100K per well to form neuron spheres. After 4 days, the spheres were transformed to low attachment 10 cm dishes and placed on a shaker for long-term culture and maturation in cortical neuron medium containing NB/N2/B27/Glu/NEAA, ascorbic acid (200 μM, Sigma, 4034-100g), dbcAMP (500 μM, Sigma, Cat #D0627), BDNF (20 ng/mL, R&D, Cat #248-BDB) and GDNF (20 ng/mL, Peprotech, Cat #450-10)⁵¹. Organoids were cultured in suspension for 90 days before adding viruses. For protocol, see dx.doi.org/10.17504/protocols.io.3byl4wrzovo5/v1.

### Open Field Test (OFT)

Mice were tested in the OFT bi-weekly. The open-field apparatus consisted of four square arenas (27 cm×27 cm), with a camera (EverFocus, EQ700) placed 1.83 μm above the floor of the arenas. EthoVision XT was used to capture and subsequently analyze animal locomotion. Each trial consisted of a 2 min habituation period, followed by a 10 min test period. To avoid confounds due to odors from non-cagemates, only animals from the same cage were recorded simultaneously. Behavior equipment was disinfected and deodorized between each animal. The distance traveled over the course of the experimental period was determined. For protocol, see dx.doi.org/10.17504/protocols.io.5qpvo972xv4o/v1.

### Beam Crossing Assay

To measure skilled locomotion using the narrowing beam assay, a clear plexiglass beam consisting of three 25 cm segments (widths 3.5 cm, 2.5 cm, and 1.5 cm) was elevated above the table surface using empty clean cages. At the narrow end, an empty cage was placed on its side, and bedding from the animal's home cage was placed inside. A white light was also placed over the broad end to motivate animals to move across the beam. For each trial, animals were placed at the end of the widest segment, with all 4 limbs touching the beam surface. Each trial was recorded with a video camera placed to the side and perpendicular to the beam's length, affording a view of both left and right hindlimbs. A trial was considered complete once the animal had traversed the beam, without turning around, and entered the goal cage. Once an animal had completed three trials, the session was completed. Beam crossing was analyzed using BORIS software, measuring the time taken to cross the beam and the number of foot slips. For protocol, see dx.doi.org/10.17504/protocols.io.n921dr2jxg5b/v1.

### Target Harvesting and Processing

Animals were transcardially perfused with 30 mL of ice-cold heparinized 1×PBS, and brains were dissected. One hemisphere of the brain was used for RNAseq. For analysis of fluorescent protein expression, one hemisphere of the brain was submerged in ice-cold 4% PFA formulated in 1×PBS and fixed for 24-48 hr at 4° C. Brains were subsequently cryoprotected at 4° C. in a solution containing 30% (w/v) sucrose for 72 hr. Brains were flash-frozen in O.C.T. Compound (Scigen, Cat #4586) using a dry ice-ethanol bath and kept at −80° C. until sectioning. For protocol, see dx.doi.org/10.17504/protocols.io.ewov1d5xovr2/v1.

### Immunostaining of Mouse Tissue

Brains were sliced at 100 μm using a cryostat (Leica Biosystems, CM1950) and collected in 1×PBS. Sections were stored in PBS supplemented with 0.02% Azide. For protocol, see dx.doi.org/10.17504/protocols.io.x54v9rkzpv3e/v1.

For immunostaining, brain sections were blocked with 5% normal donkey serum in 0.1% PBST for 90 mins, then incubated with primary antibody solution prepared in 0.1% PBST supplemented with 5% normal donkey serum overnight on a shaker at 4° C. After washing in 0.1% PBST (3×30 min), the sections were incubated with secondary antibodies overnight on a shaker at 4° C. and then washed 3×30 min in 0.1% PBST. The tissues were mounted on glass slides with Prolong Diamond Antifade mounting media (Thermo Fisher Scientific, Cat #P36970). Imaging was performed with a spinning disk confocal microscope. For protocol and antibodies, see dx.doi.org/10.17504/protocols.io.kqdg3q7ypv25/v1.

### RNA Extraction

RNA extraction from cells was performed using the Direct-zol RNA Miniprep kit (Zymo, Cat #R2051). Briefly, the cells were removed from incubator, washed once with DPBS, then 350 μL of TRIzol was added directly to each well to collect the cells. Subsequent RNA extraction was done following the manufacturer's instructions. For protocol, see dx.doi.org/10.17504/protocols.io.6qpvr9r2zvmk/v1.

RNA extraction from organoids was performed using the QIAshredder kit (Qiagen, Cat #79654) and miRNeasy Mini Kit (Qiagen Cat #217004). Briefly, 3 organoids were harvested for each RNA extraction and 350 μL of Buffer RLT from RNeasy Mini Kit+3.5 μL β-mercaptoethanol were added to lyse the organoid. Subsequent RNA extraction was done following the manufacturer's instructions. For protocol, see dx.doi.org/10.17504/protocols.io.8lwgbr95qlpk/v1.

RNA extraction from mouse brain tissue was performed using the miRNeasy Mini Kit (Qiagen catalog, Cat #217004) on a QIAcube Connect (Qiagen) After perfusion with cold PBS, the entire olfactory bulb was dissected and placed in TRIzol directly and processed immediately for RNA extraction following manufacturer's instructions. For protocol, see dx.doi.org/10.17504/protocols.io.eq2ly618pgx9/v1.

### RNA Sequencing (NA-seq)

Bulk RNAseq was performed by the Millard and Muriel Jacobs Genetics and Genomics Laboratory at the California Institute of Technology. RNA integrity was assessed using the RNA 6000 Pico Kit for Bioanalyzer (Agilent Technologies, Cat #5067-1513), and mRNA was isolated from −1 g of total RNA using the NEBNext Poly(A) mRNA Magnetic Isolation Module (NEB, Cat #E7490). RNA-seq libraries were constructed using the NEBNext Ultra II RNA Library Prep Kit for Illumina (NEB, Cat. No./ID: E7770) following the manufacturer's protocols. Briefly, mRNA was fragmented to an average size of 200 nt by incubating at 94° C. for 15 min in the first strand buffer. cDNA was then synthesized using random primers and ProtoScript II Reverse Transcriptase followed by second strand synthesis using NEB Second Strand Synthesis Enzyme Mix. The resulting DNA fragments were end-repaired, dA-tailed and ligated to NEBNext hairpin adaptors (NEB, Cat. No./ID: E7335). Following ligation, adaptors were converted to the “Y” shape by treating with USER enzyme, and DNA fragments were size selected using Agencourt AMPure XP beads (Beckman Coulter #A63880) to generate fragment sizes between 250-350 bp. Adaptor-ligated DNA was PCR amplified followed by AMPure XP bead clean up. Libraries were quantified using a Qubit dsDNA HS Kit (ThermoFisher Scientific, Cat. No./ID: Q32854), and the size distribution was confirmed using a High Sensitivity DNA Kit for Bioanalyzer (Agilent Technologies, Cat. No./ID: 5067-4626). Libraries were sequenced on an Illumina NextSeq2000 in paired end mode with a read length of 50 nt and sequencing depth of 25 million reads per library. Base calls and FASTQ generation were performed with DRAGEN 4.2.7.

### Transcriptomics Data Analysis

Raw sequencing reads were processed and aligned by Rsubread package v2.16.1 (bioconductor.org/packages/release/bioc/html/Rsubread.html, RRID:SCR_016945) in R (v4.3.2) to align trimmed reads. For human cortical organoids, reads were aligned to the GRCh38.p14 reference genome, while mouse brain tissues were aligned to the GRCm39 reference genome. The corresponding gene annotation files (GTF) were used with featureCounts (subread.sourceforge.net/featureCounts.html, RRID:SCR_012919) to generate gene level counts for each dataset. Data were subsequently normalized with DESeq2 v1.42.1 (bioconductor.org/packages/release/bioc/html/DESeq2.html, RRID:SCR_015687) and principal coordinate analysis was subsequently performed by multidimensional scaling to assess sample level clustering using the limma package (v3.60.4, RRID:SCR_010943). DESeq2 was then used to assess differential gene expression between rtTA and SAS3 samples. A two-tailed false discovery rate (FDR)<0.05 (original Benjamini-Hochberg method) determined statistical significance and |log₂(FC)|>1.0 was selected to identify differentially expressed genes (DEGs). Pathway analysis by statistical overrepresentation was performed with DEGs separately for upregulated and downregulated genes with the Gene Ontology (GO) database using the Rapid Integration of Term Annotation and Network (RITAN) package v1.26.0577 (bioconductor.org/packages/release/bioc/html/RITAN.html). A two-sided adjusted p-value (q-value) threshold of 0.05 (Benjamini-Hochberg method) was used to determine statistically overrepresented pathways.

### Image Analysis

Images were processed and analyzed with imageJ2 V2.14.0/1.54f The “Analyze Particles” and “Nucleus Counter” plugins were used for aggregate size and number analysis; “Analyze Skeleton” was used for primary neuron neurite length quantification. The fluorescence intensity measurement was used to determine striatal TH projection intensity.

### Quantification and Statistical Analysis

Data are presented as mean±SEM and were derived from at least three independent experiments. Information on replicates (n) is given in figure legends. Statistical analysis was performed using the unpaired t-test, also known as Student's t-test (comparing two groups) or ANOVA with Dunnett test (comparing multiple groups against control). Distributions of raw data approximated normal distributions.

### Example 2

### Programmable Self-Assembling System to Model Intracellular Protein Aggregation and Decode Neurodegenerative Diseases

This example demonstrates a genetically-encoded, modular platform that enables inducible, tunable, and cell-type-specific formation of intracellular protein aggregates across diverse in vitro and in vivo experimental models. Using this platform, self-assembling variants of αSyn, Tau, and TDP-43 were engineered to model hallmark features of PD, AD, and ALS, respectively.

In HEK cells, primary neurons and human brain organoids, self-assembling αSyn (SAS), Tau (SA-Tau) and TDP-43 (SA-TDP) induced histopathology that closely mirrored that in the human diseases, establishing a new paradigm in disease modeling. SAS aggregates formed LB-like inclusions that interacted with mitochondria, lipids, lysosomes, and ubiquitin, features observed in PD patients but not fully captured by prior experimental models. Applying the same approach to Tau produced hyperphosphorylated tangles that were highly ubiquitinated, elevated neurofilament levels, and promoted extracellular amyloid beta production, resulting in severe neurodegeneration. In contrast, self-assembling TDP-43 formed phosphorylated cytoplasmic inclusions that exhibited minimal interaction with mitochondria and ubiquitin, leading to milder neurodegeneration at comparable expression levels and time. Furthermore, non-invasive, cell-type-specific delivery of SAS to DA neurons in the mouse brain led to motor deficits, DA neuron loss, reduced striatal projections, and propagation of αSyn aggregates from the substantia nigra pars compacta (SNpc) to other brain regions. This system not only recapitulates key histological and transcriptional features of human disease but also enables programmable modeling of aggregate biology across diverse contexts. This platform provides a powerful, extensible tool to interrogate mechanisms of neurodegeneration and to accelerate therapeutic development.

### Self-Assembling System Induces Intracellular Protein Aggregates and Enables Tunable Proteinopathy

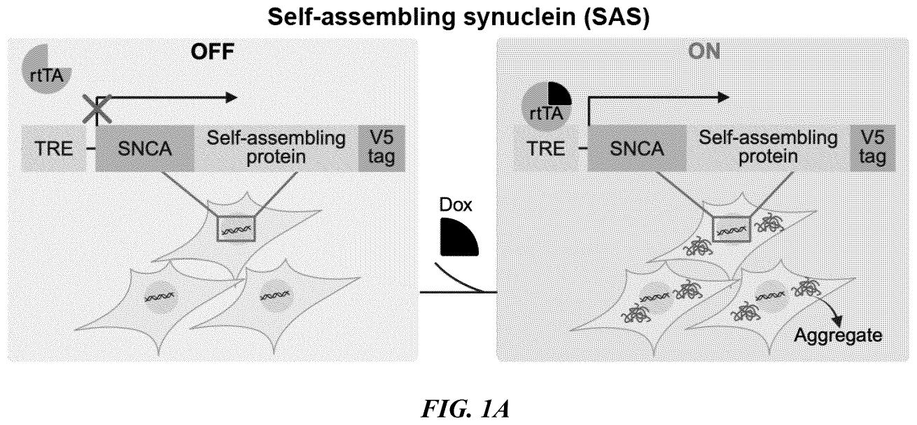

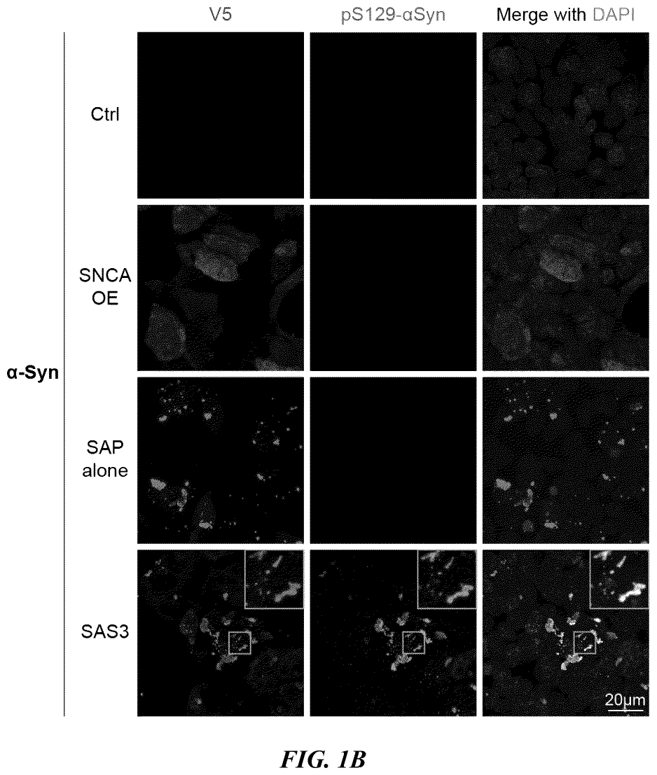

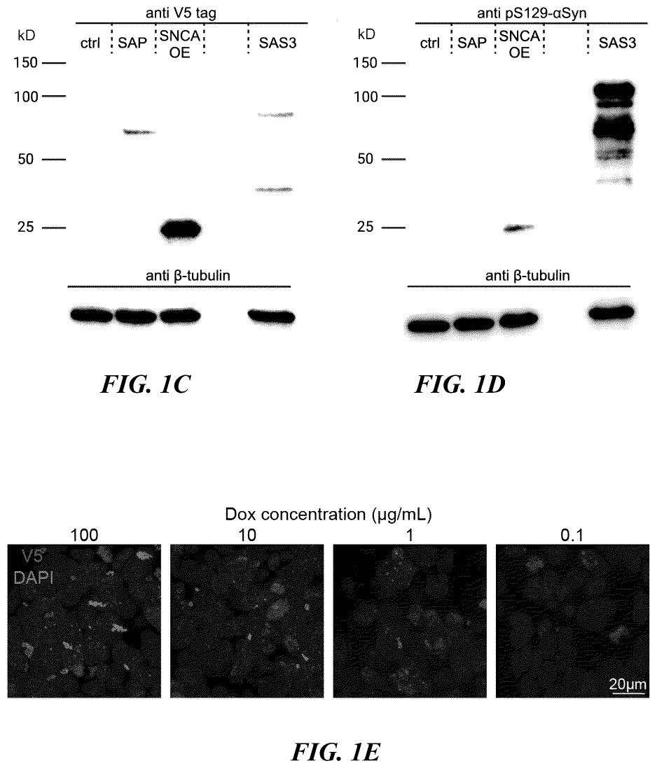

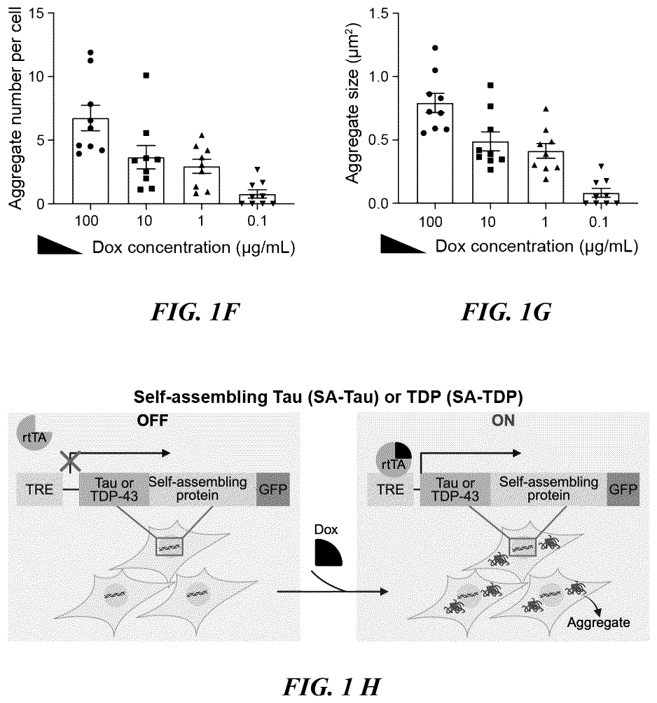

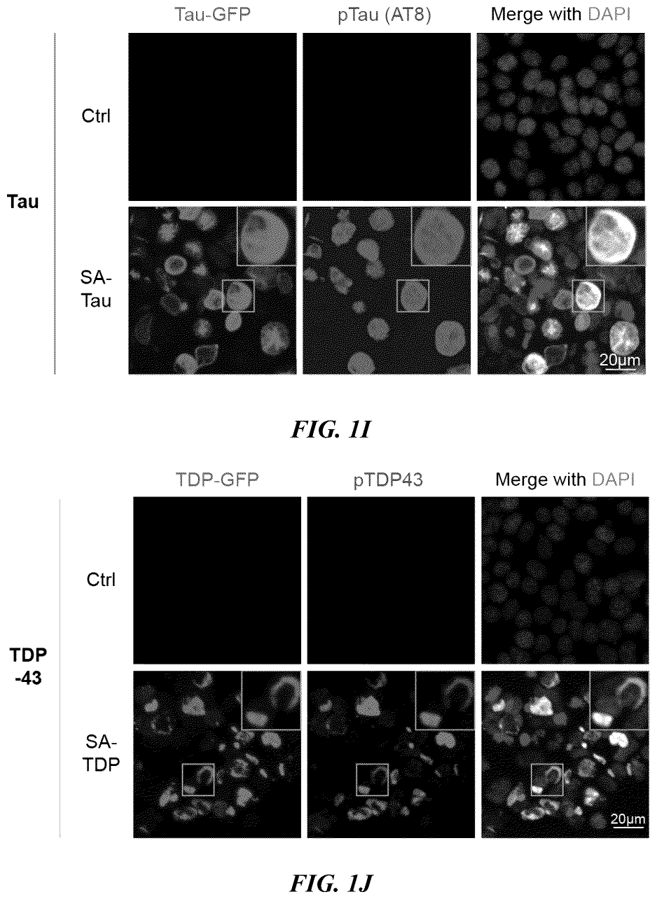

Abnormal protein aggregates in neurodegenerative diseases are composed of not only misfolded protein, but also diverse cellular components. Rather than synthesizing pure protein fibrils in solution and then introducing them into cells, inducing protein aggregation intracellularly would enable the aggregates to incorporate endogenous cellular components, therefore more faithfully recapitulating disease pathology. Previous studies have shown that de novo-designed helical filaments and naturally occurring self-assembling proteins can form ordered aggregates spontaneously in solution or in yeast. Such self-assembling proteins have been applied to record protein activity in vivo. Inspired by these studies, the instant application developed a genetically encoded platform for programmable aggregate formation. Focusing initially on αSyn, a library of self-assembling synuclein (SAS) constructs was designed, each consisting of human SNCA, the gene encoding αSyn, fused with a different self-assembling protein (SAP) at a position predicted to remain accessible on the exterior surface of the resulting filament (Table 3). To enable temporal control, this design was integrated into a doxycycline-regulated system. Cell-type specificity was achieved by utilizing custom promoters to drive expression of the reverse tetracycline-controlled transactivator (rtTA), allowing for targeted induction of αSyn aggregation in desired cell populations (FIG. 1A).

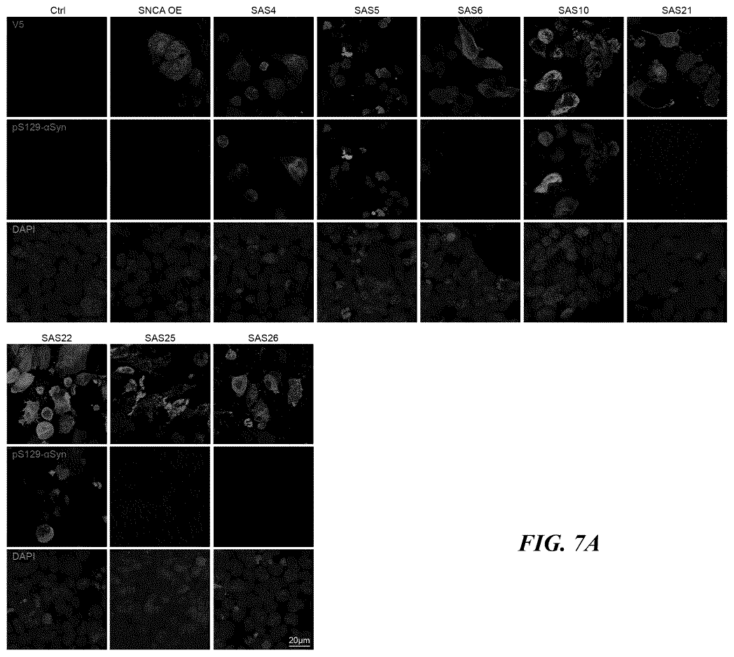

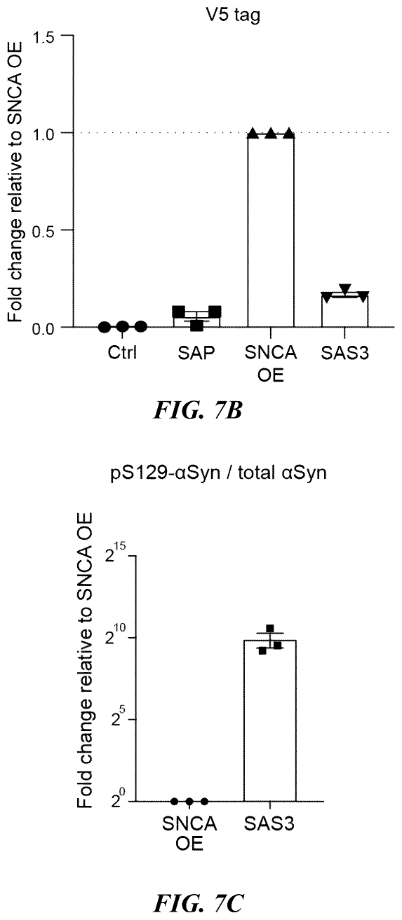

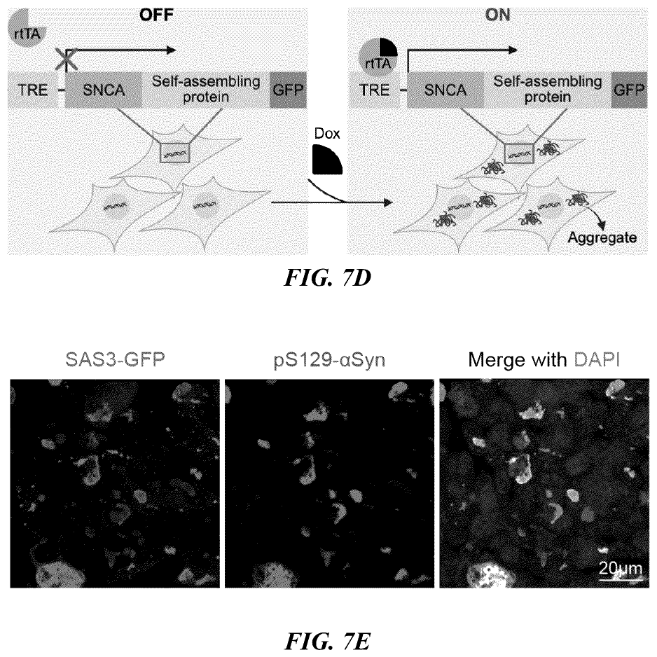

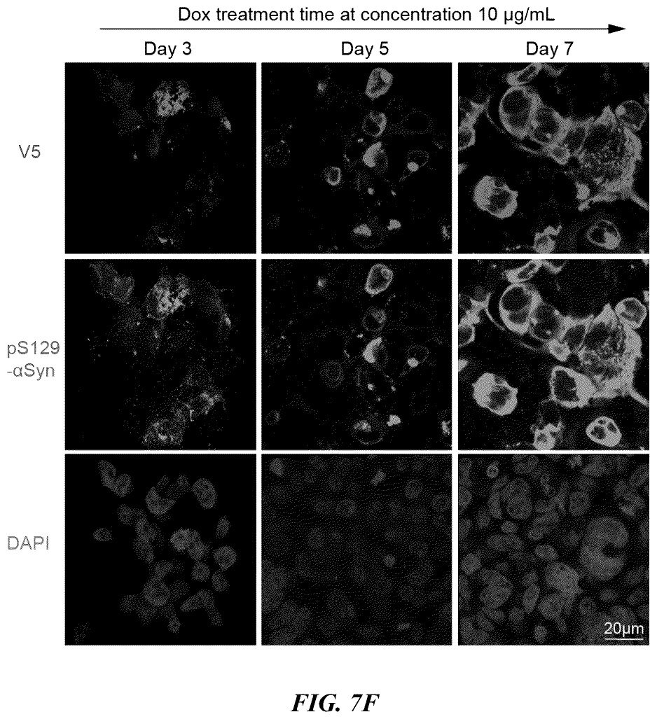

In vitro screening in HEK cells identified several constructs that not only formed αSyn aggregates but also induced substantial Ser129 phosphorylation of αSyn (pS129-αSyn), a hallmark of PD pathology (FIG. 1B and FIG. 7A). Different SASs resulted in different types of aggregates with varying ability to induce pS129-αSyn. Among these, SAS3 formed condensed punctate structures and robustly induced pS129-αSyn. To confirm that the pS129-αSyn induction was specific to the αSyn aggregates, SAS3 was compared with rtTA-transfected control (Ctrl), SNCA overexpression (SNCA OE), and SAP alone. Only SAS3 led to strong pS129 induction; Neither αSyn OE nor SAP alone triggered similar pathology (FIG. 1B). Western blot analysis revealed that SAS3 induced 1000-fold higher pS129-αSyn compared to SNCA OE (FIGS. 1C and 1D and FIGS. 7B and 7C), underscoring the pathological potency of the construct. To enable real-time visualization of aggregate dynamics, we generated a fluorescent version of SAS3 by replacing the V5 epitope tag with a GFP reporter (SAS-GFP) (FIGS. 7D and 7E). Live imaging demonstrated gradual appearance and growth of intracellular αSyn aggregates. The SAS system was designed to allow precise control of αSyn aggregation and pathology through manipulation of doxycycline (dox) concentration and treatment duration. Indeed, titration of dox concentration decreased the average number and size of aggregates (FIGS. 1E, 1F, and 1G). Prolonged dox treatment led to a corresponding increase in aggregation and αSyn phosphorylation (FIG. 7F).

To assess the generalizability of the self-assembling protein platform beyond synuclein, next analogous constructs were developed targeting two other major neurodegenerative disease proteins: Tau and TDP-43, which are central to AD and ALS, respectively. Using the same modular design as SAS, Tau (2N4R isoform) or TDP-43 was fused to the self-assembling domains used in SAS3 (FIG. 111; Tables 4-5). In HEK cells, self-assembling Tau (SA-Tau) formed fibrillar cytoplasmic tangles that were positive for phospho-Tau (AT8), a hallmark of tauopathy and AD (FIG. 1I). Self-assembling TDP-43 (SA-TDP) induced cytoplasmic inclusions and mislocalization of TDP-43 with strong phospho-TDP-43 signal, mirroring ALS pathology (FIG. 1J).

Together, these results demonstrate that the self-assembling system demonstrated herein enables intracellular formation of protein aggregates, relevant to multiple neurodegenerative diseases. Its compatibility with genetic control, tunability, and modularity provide robust, scalable, and refined preclinical models for studying disease mechanisms and developing therapeutics.

### Self-Assembling αSyn, Tau, and TDP-43 Aggregates Recapitulate Distinct, Protein-Specific Histopathology in Neurons

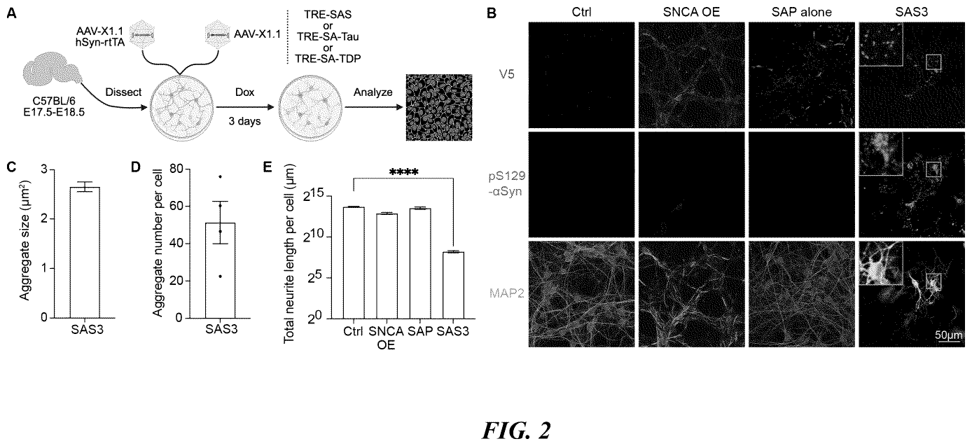

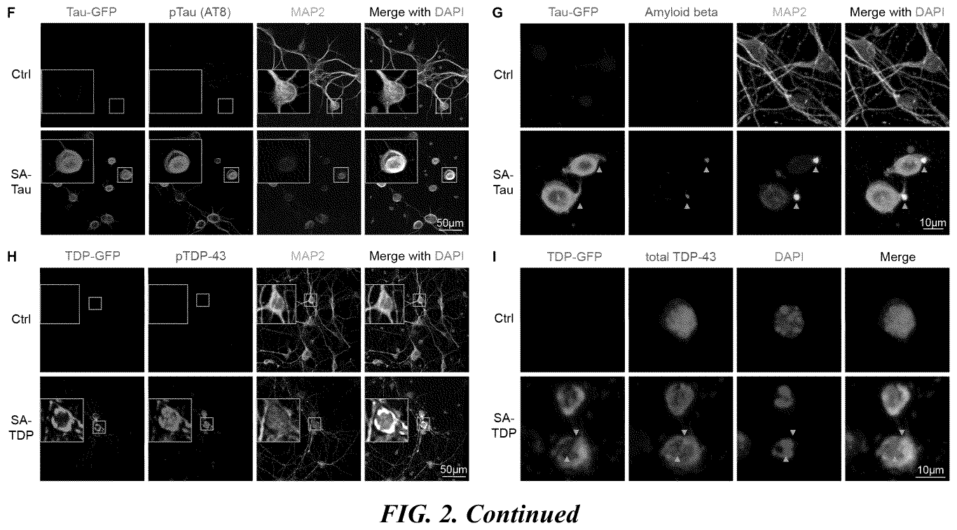

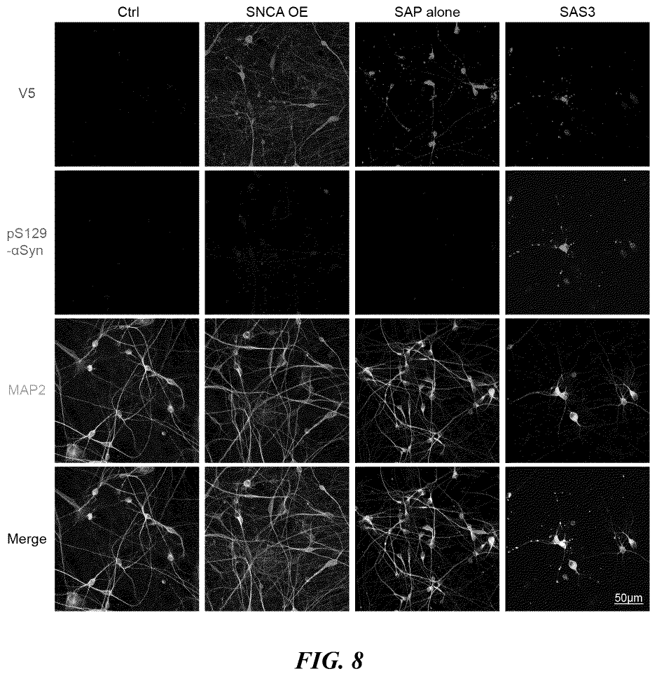

Another defining feature of neurodegenerative diseases is neuronal degeneration. Having shown that the self-assembling system drives robust, phosphorylated protein aggregation in HEK cells, next whether these aggregates could trigger neuronal degeneration was investigated. The self-assembling system was delivered to mouse primary neurons using adeno-associated virus (AAV) AAV9-X1.1, an engineered AAV that efficiently transduces primary murine and human neurons in culture (FIG. 2, panel A). As observed in HEK cells, SAS3 induced prominent αSyn aggregation and pS129-αSyn in both the soma and neurites of primary neurons (FIG. 2, panel B and quantified in FIG. 2, panels C-D). Notably, SAS3-transduced neurons also exhibited pronounced neurite retraction after 3 days of dox treatment (FIG. 2, panels B and E), mimicking the neurodegeneration observed in PD. Consistent with these findings, SAS3 also induced αSyn aggregation and neurite retraction in human embryonic stem cell (hESC)-derived neurons (FIG. 8). Similarly, SA-Tau formed extensive tau tangles positive for phospho-Tau (AT8), accompanied by severe neuronal loss and neurite degeneration as marked by MAP2 (FIG. 2, panel F). Intriguingly, SA-Tau expression also led to elevated levels of extracellular amyloid beta (A3) (FIG. 2, panel G), suggesting a potential link between intracellular Tau aggregation and Aβ dysregulation. Neurons expressing SA-TDP exhibited cytoplasmic TDP-43 inclusions with strong pTDP-43. However, compared with equivalent expression of SAS and SA-Tau, SA-TDP induced milder neurodegeneration (FIG. 2, panel H). Total TDP-43 staining revealed translocation of endogenous TDP-43 from the nucleus to cytoplasm and aggregation (FIG. 2, panel I). Collectively, these findings establish that the self-assembling protein aggregates not only induce neurodegeneration, but also recapitulate distinct, protein-specific pathological signatures.

### Self-Assembling System Reveals Distinct Organelle Interactions and Pathological Signatures of αSyn, Tau, and TDP-43 Aggregates in Human Brain Organoids

How different protein aggregates drive distinct neurotoxicity remains poorly understood. It is hypothesized that differential interactions between protein aggregates and other cellular components may underlie their varying toxic effects, contributing to the pathogenesis of different neurodegenerative diseases. Supporting this idea, postmortem examination of PD patients has shown that aggregates are far from homogeneous lumps of toxic protein. LBs often contain hundreds of different proteins and even entire membranous organelles tangled up in them. One considerable advantage of the self-assembling system is its ability to drive intracellular aggregation, potentially enabling incorporation of other cellular components and better recapitulation of the molecular complexity of pathological inclusions.

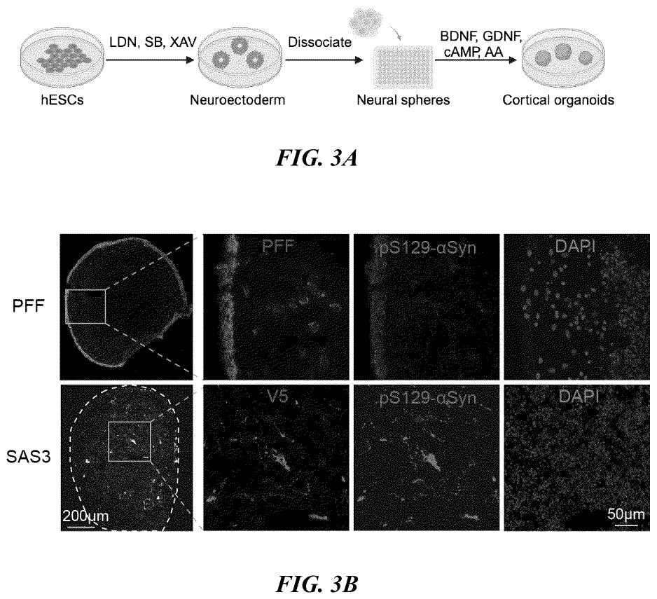

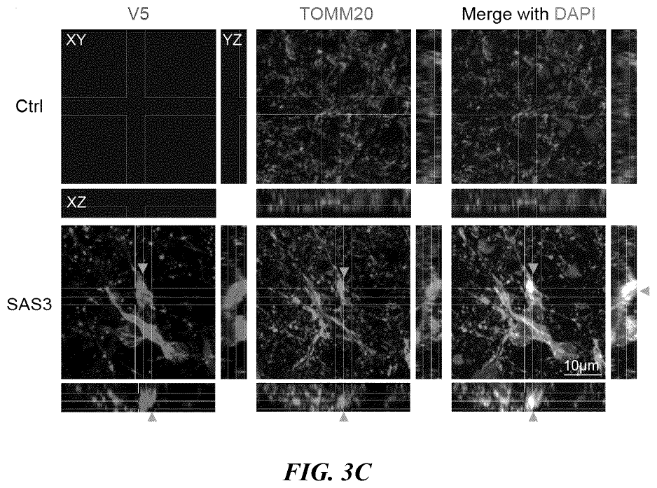

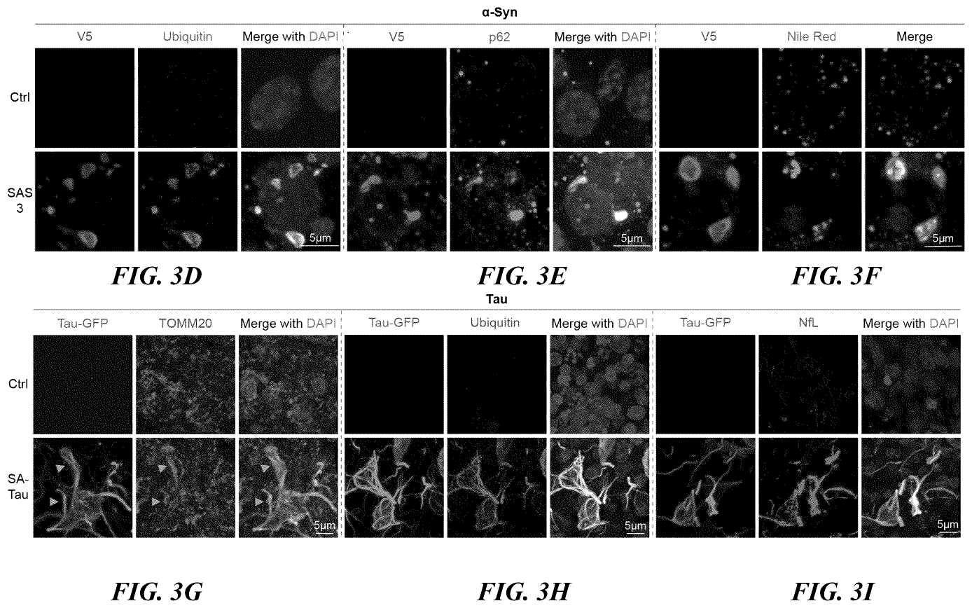

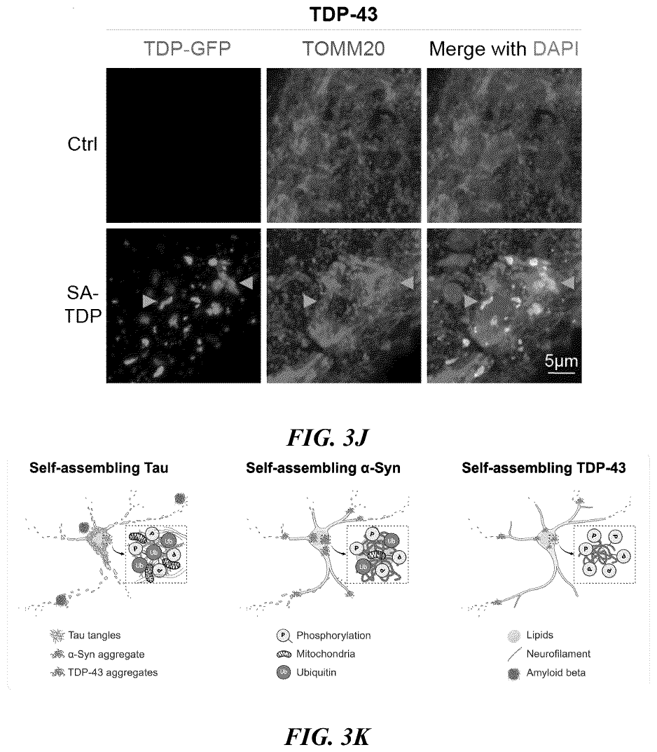

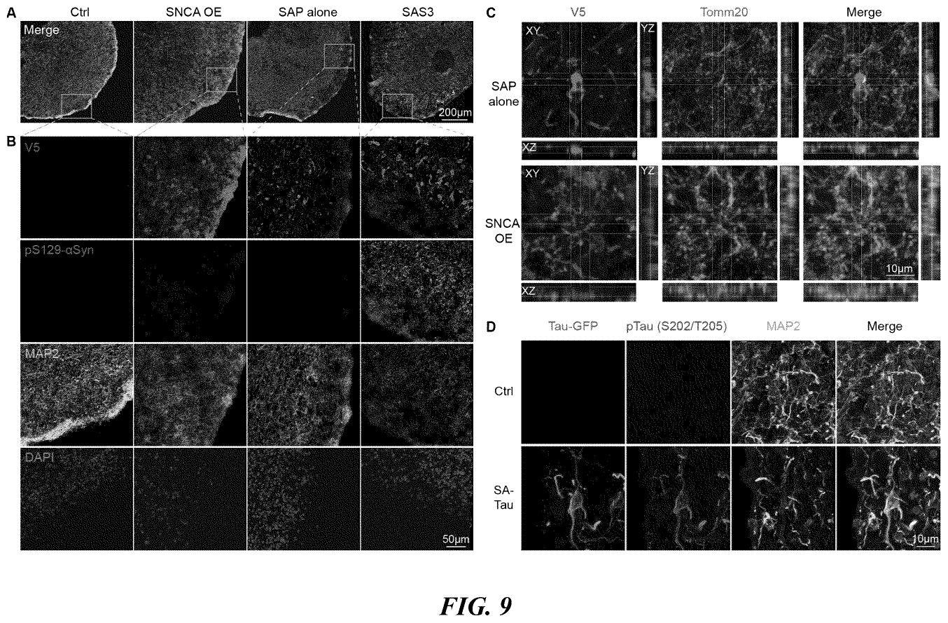

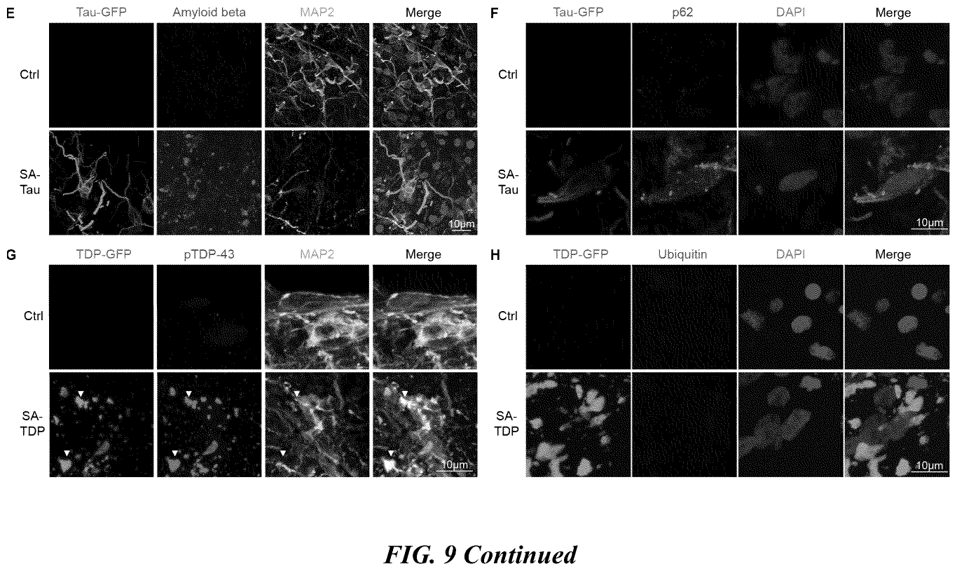

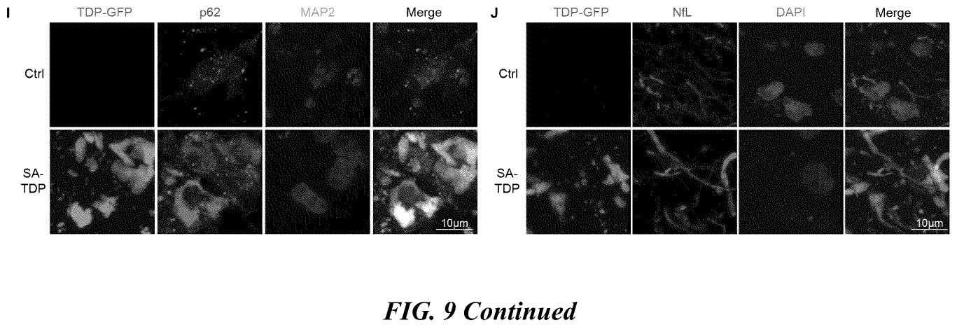

To test this hypothesis in a physiologically relevant context, SAS, SA-Tau and SA-TDP43 were delivered to hESC-derived brain organoids using AAV-X1.1 (FIG. 3A). Organoids produce diverse cell types in a 3D environment that closely mimics the function and structure of in vivo counterparts. While organoids have been widely used for modeling neurodevelopment, existing organoid models often fail to display hallmark features of neurodegenerative diseases, such as LB formation and neuronal degeneration in PD. In particular, αSyn PFFs, which have shown promise in animal studies, are inefficient in organoids due to limited uptake and poor tissue penetration (FIG. 3B). In contrast, AAV-delivered SAS3 was able to efficiently access the entire organoids. Immunostaining for the V5 tag and pS129-c-Syn revealed widespread synucleinopathy, with aggregates present even in deep layers of organoids (FIG. 3B and FIG. 9, panels A and B). Similarly, SA-Tau and SA-TDP produced hallmark pathologies of AD and ALS in human brain organoids, including phosphorated Tau tangles and TDP-43 aggregates, extracellular Aβ accumulation, and cytoplasmic TDP-43 inclusions (FIG. 9, panels D-G). Together, these findings establish the self-assembling system as a robust platform for modeling and investigating authentic inclusion biology in a human-relevant system, filling a major gap in current disease models.

To investigate the molecular composition and cellular impact of aggregates, confocal Z-stack imaging of organoids infected with self-assembling constructs and stained for cellular components was performed. In SAS-expressing organoids, co-staining for the V5 tag to trace aggregates and the mitochondrial membrane marker TOMM20 revealed clear TOMM120 signal within the αSyn aggregates, indicating that mitochondria were sequestered in the inclusions (FIG. 3C), a feature not observed in control groups (FIG. 9, panel C). SAS-induced inclusions were highly ubiquitinated (FIG. 3D) and showed prominent accumulation of autophagy adaptor p62/SQSTM1 in and around the aggregates (FIG. 3E), suggesting these inclusions are recognized and targeted by protein clearance pathways. Nile red staining revealed that aggregates were enriched in lipids (FIG. 3F), a common feature of LBs from PD patient brains. Tau and TDP-43 aggregates displayed distinct aggregate-organelle interaction profiles. In SA-Tau-expressing organoids, mitochondria accumulated around the aggregates rather than being trapped inside (FIG. 3G). Tau tangles were also highly ubiquitinated (FIG. 3H), p62-positive (FIG. 9, panel F) and colocalized with neurofilament light chain (NfL) (FIG. 3I). In contrast, SA-TDP-induced aggregates showed minimal co-localization with mitochondria, ubiquitin, or NfL, but still induced p62 accumulation (FIG. 3J and FIG. 9, panels H-J). This divergence in aggregate-organelle interactions (FIG. 3K) may explain the milder neurodegeneration observed in SA-TDP-expressing organoids and primary neurons compared to SAS and SA-Tau.

Self-assembling protein system recreated protein-specific aggregation and disease-relevant pathology in human brain organoids and uncovered distinct aggregate-organelle interaction profiles that might underlie differential aggregates toxicity. The ability to resolve these aggregate-specific signatures provides a unique opportunity to dissect the molecular basis of selective vulnerability in PD, AD, ALS, and other related disorders.

### SAS Forms LB-Like Inclusions and Nucleate Aggregation of Endogenous αSyn, Triggering Neuronal Inflammation and Death

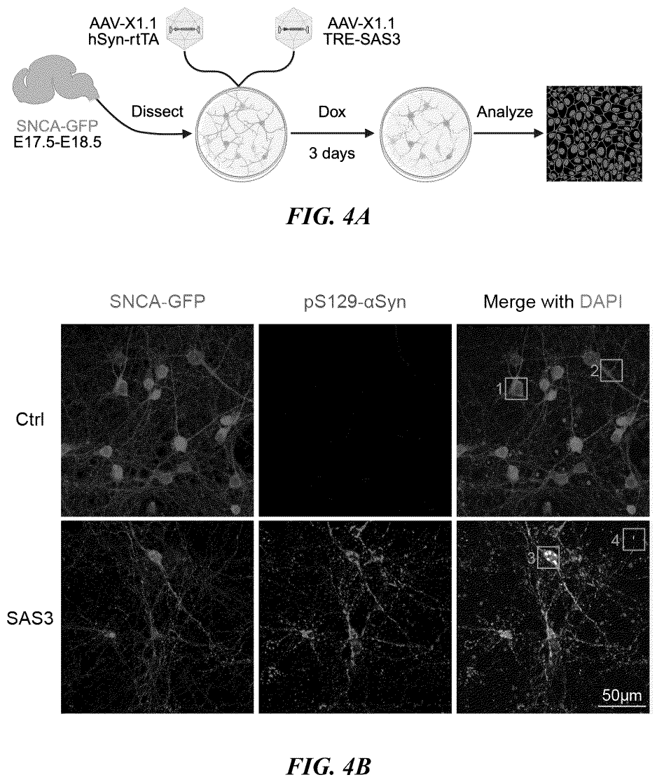

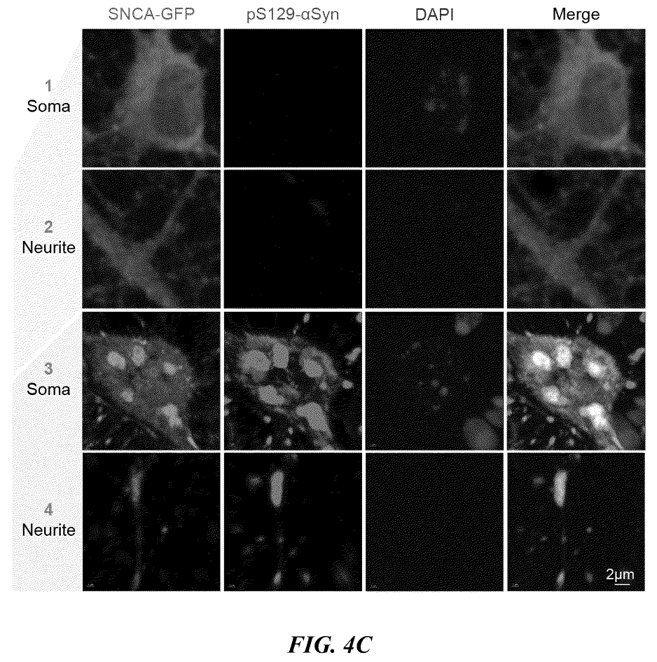

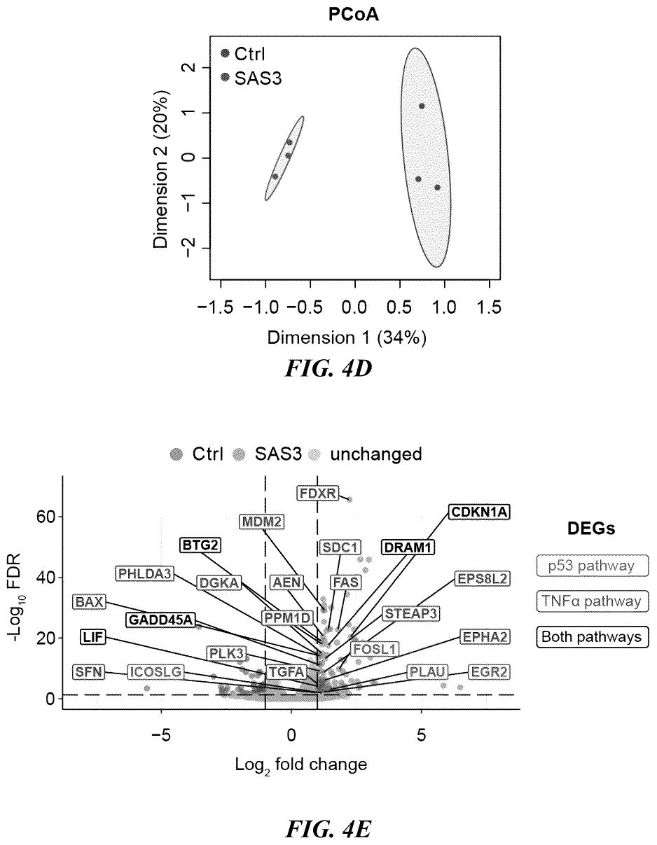

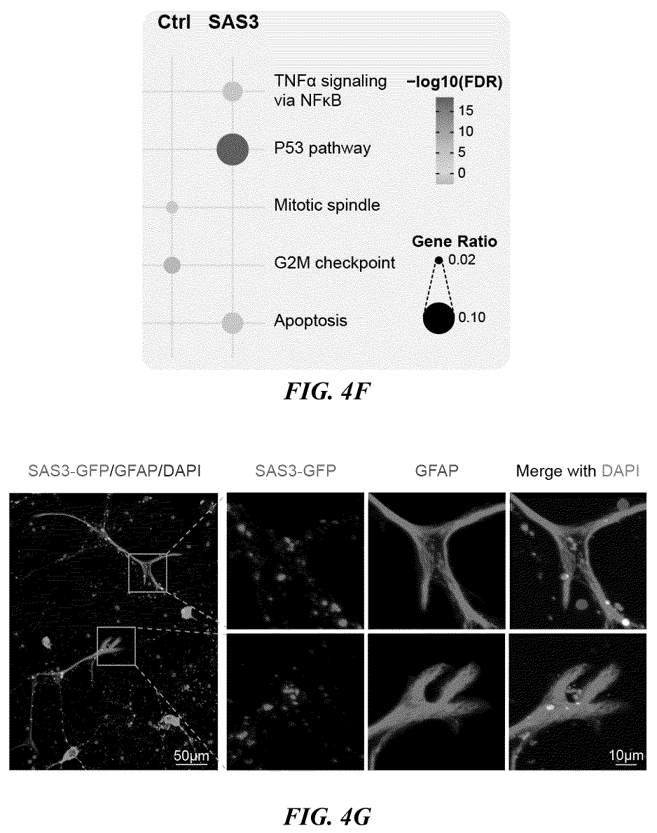

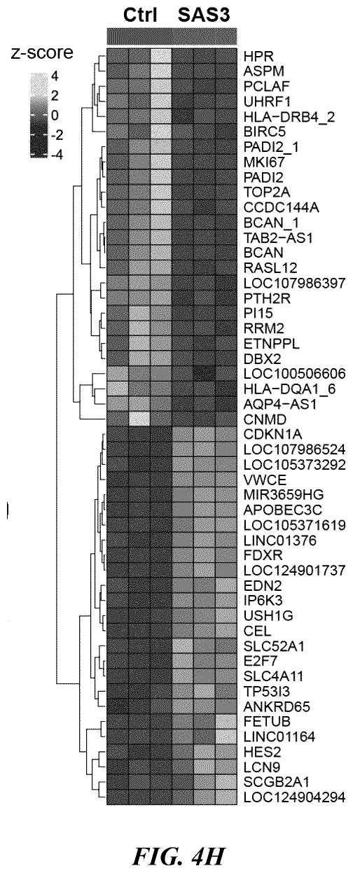

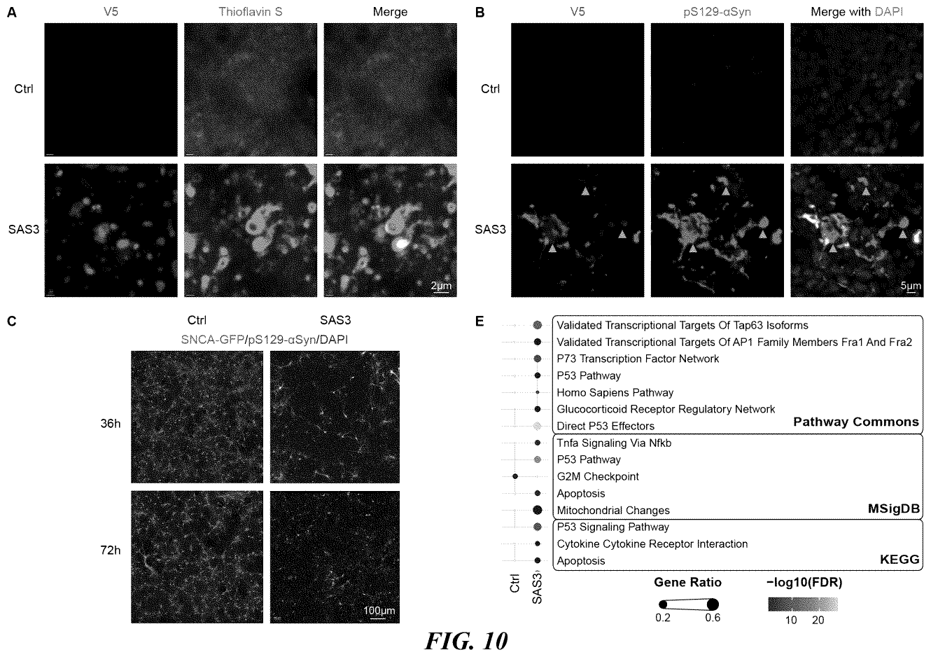

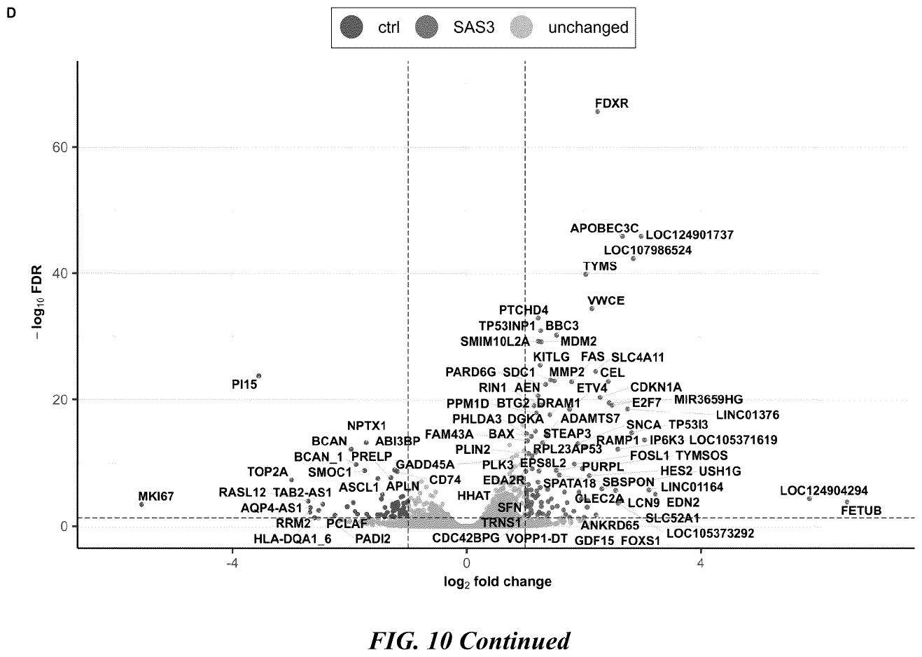

Having established the self-assembling system faithfully recapitulates protein-specific pathology across neurodegenerative diseases, Applicant next focused on PD to further explore the full potential of SAS in elucidating mechanisms of αSyn-mediated neurodegeneration. The present findings demonstrated that SAS induced complex, multi-component inclusions that closely resemble human LBs in terms of molecular composition (FIGS. 3C-F). This section further showed the inclusion also recapitulate key morphological features of LBs. Thioflavin S staining confirmed the presence of p-sheet-rich fibrillar content in SAS-expressing neurons (FIG. 10, panel A). Notably, the Thioflavin S signal often extended beyond the V5-positive core, suggesting the existence of secondary aggregated species. Co-staining of the V5 tag and pS129-αSyn revealed numerous V5-negative and pS129-αSyn-positive aggregates, pointing to the formation of endogenous αSyn inclusions (FIG. 10, panel B). PD is widely considered a prion-like disease, in which abnormal αSyn aggregates can induce secondary nucleation of normal αSyn, promoting disease spreading. To determine whether SAS-induced aggregates possess this capability, primary neurons carrying an SNCA-GFP reporter were used, enabling tracking of the endogenous αSyn (FIG. 4A). Following 36 hours of dox treatment, neurons expressing SAS3 exhibited degeneration phenotypes compared with control groups (FIG. 4B), with further worsening by 72 hours (FIG. 10, panel C). Close examination of SAS3-transduced neurons revealed endogenous αSyn aggregation in both soma and neurites (FIG. 4C), confirming a prion-like seeding effect.

To better understand the mechanisms of SAS-induced neurodegeneration, bulk RNAseq of SAS-transduced and control organoids was conducted. SAS-expressing organoids displayed a distinct transcriptomic profile (FIG. 4D), with 178 differentially expressed genes (DEGs) with adjusted p-value <0.05 and |log₂ fold change (FC)|>1 (FIG. 10, panel D). Pathway analysis revealed significant upregulation of p53 and TNFα signaling, key regulators of apoptosis and inflammation (FIG. 4E). Notably, the TNF-NF-κB-p53 axis has been demonstrated to mediate dopamine neuron survival in neuron transplant experiments. Targeting this pathway might enhance neuron survival in PD. Consistent with human transcriptomic data, SAS-treated organoids showed activation of apoptosis, inflammation, and mitochondrial dysfunction pathways (FIG. 4F and FIG. 10, panel E). In line with transcriptomic findings, immunostaining for the astrocyte marker GFAP revealed astrocyte activation surrounding SAS-expression neurons and active engagement with protein inclusions (FIG. 4G). Among the top 50 most dysregulated genes, many were previously implicated in PD (FIG. 4H). For example, FDXR, which encodes the mitochondrial ferredoxin reductase that helps produce iron-sulfur clusters, has been linked to inflammation-associated neurodegeneration, consistent with the earlier finding that SAS inclusions sequester mitochondria (FIG. 3C).

These results illustrated that SAS induced both primary and secondary αSyn aggregation, forming inclusions that resemble human LBs in molecular composition, morphology and pathogenic function. Further, these inclusions triggered TNFα-mediated neuroinflammation, glial activation and ultimately neuronal death.

### Brain-Wide Delivery of SAS Induces PD-Relevant Histopathology and Behavioral Deficits in Mice

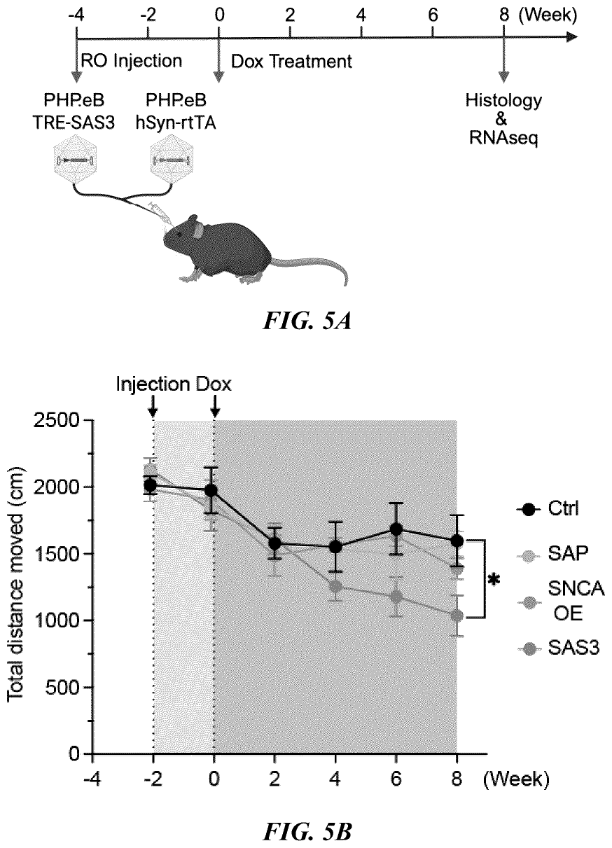

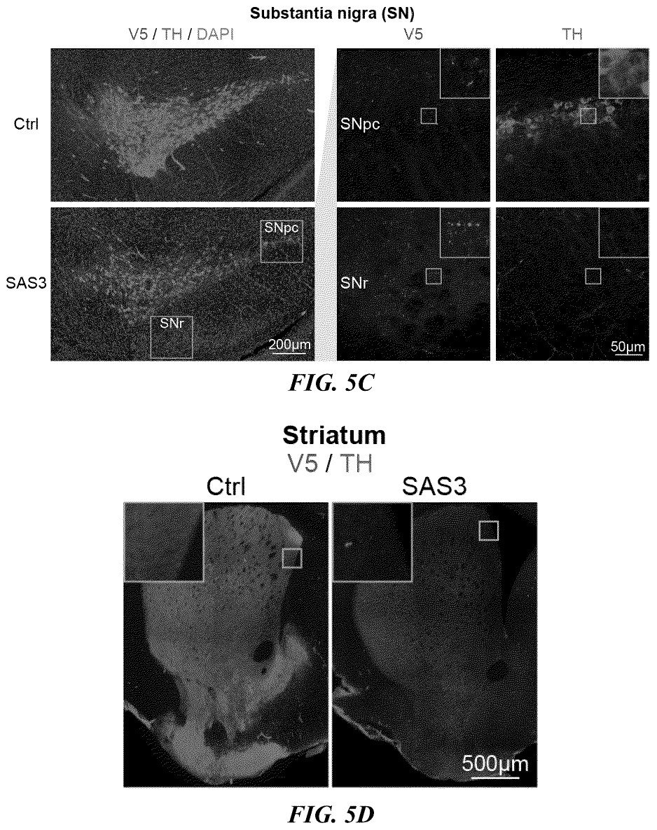

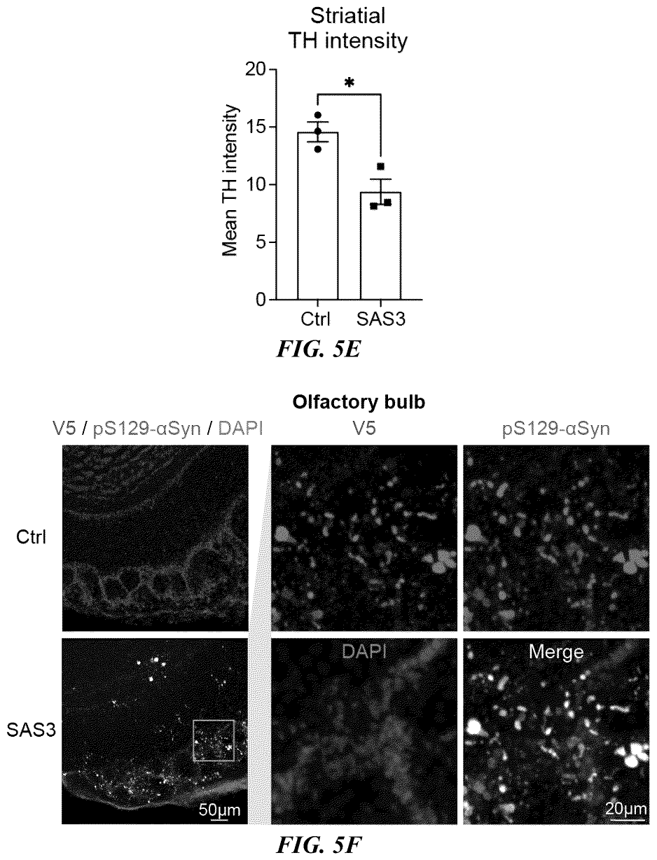

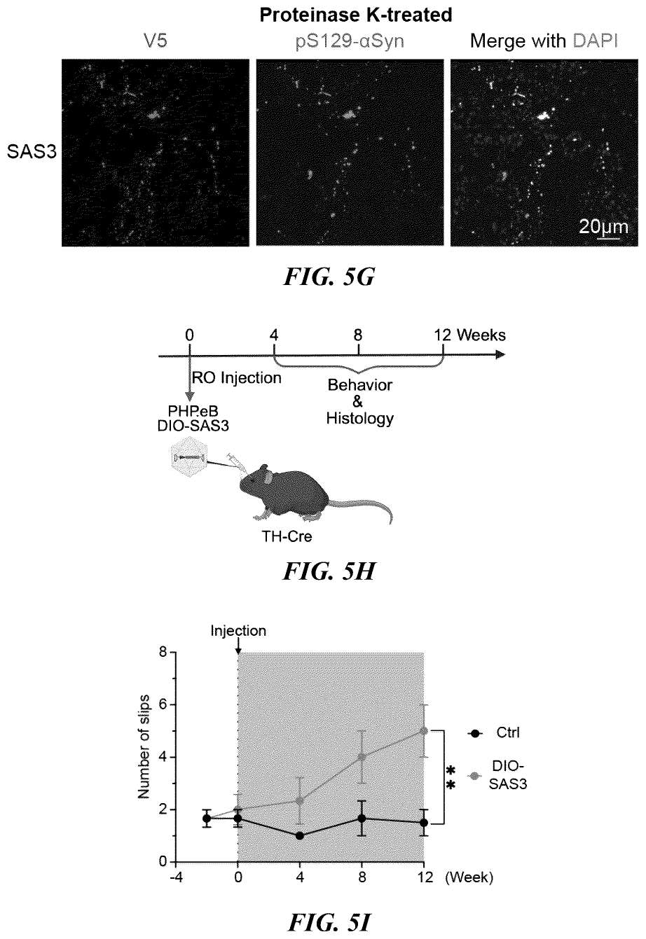

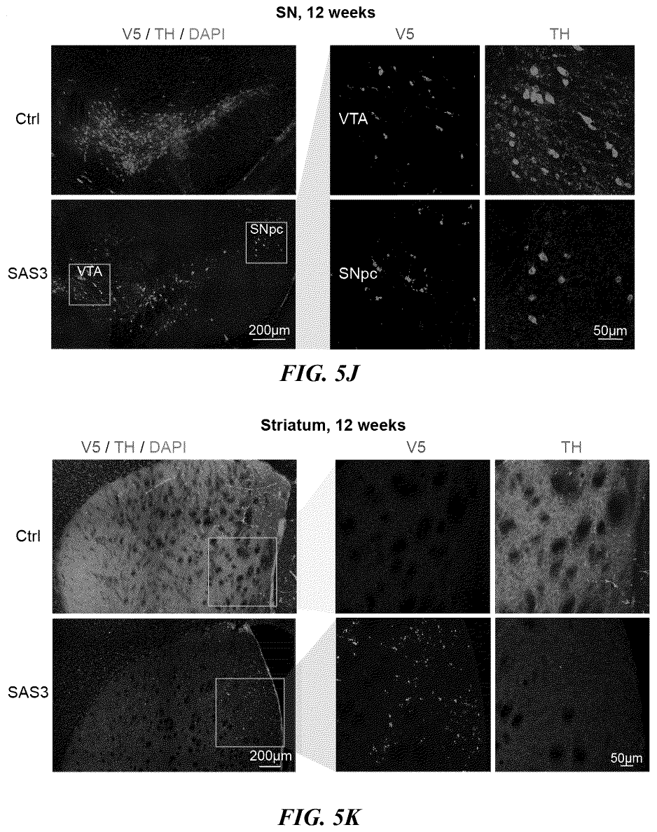

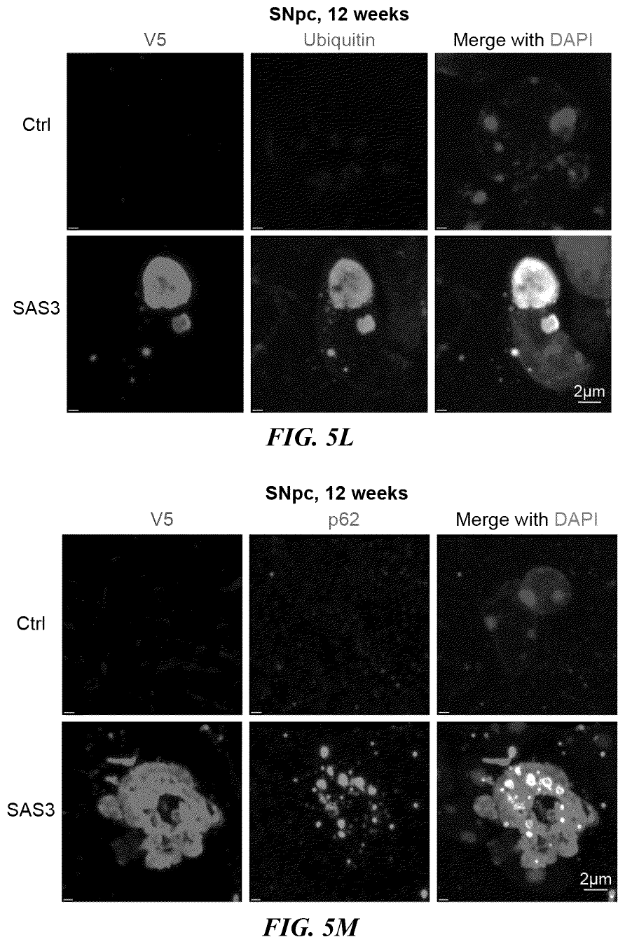

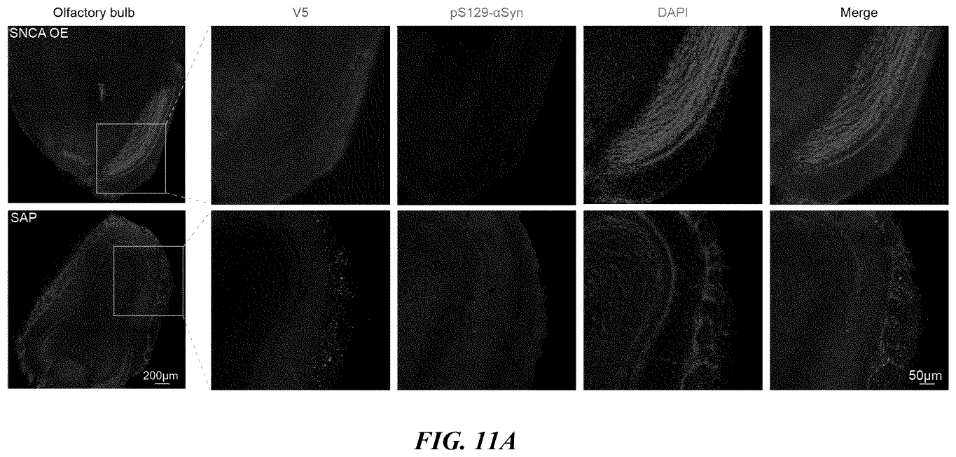

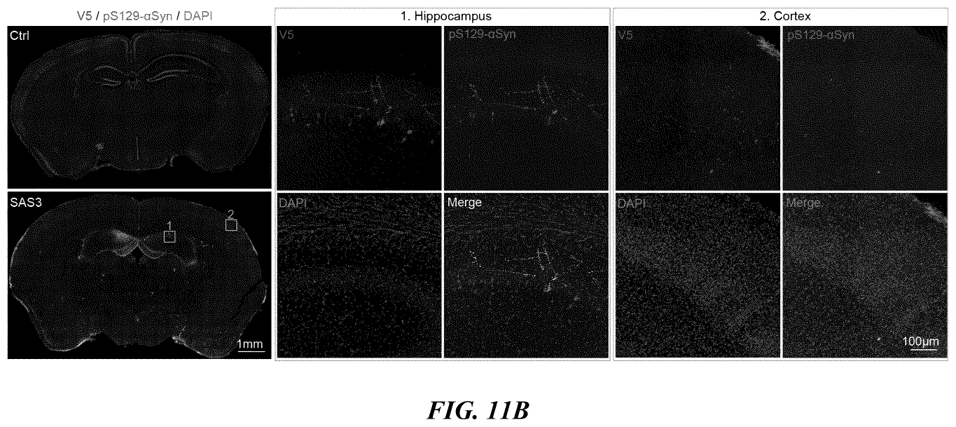

The primary clinical manifestation of PD is movement disorder. To investigate whether SAS can induce behavior abnormalities in vivo, AAV-PHP.eB, an AAV capable of crossing the blood-brain barrier (BBB) after systemic injection, was utilized to achieve non-invasive delivery of SAS into the mouse central nervous system (CNS). To specifically target neurons, the human synapsin (hSyn) promoter was used to drive the expression of rtTA. The hSyn-rtTA and TRE-SAS3 constructs were packaged into AAV-PHP.eB and co-delivered into wild-type (WT) C57BL/6J mice via retro-orbital (RO) injection. Dox treatment started 2 weeks after virus injection and open field test (OFT) assay were performed every other week (FIG. 5A). After 4 weeks of dox treatment, SAS3-treated mice exhibited a progressive decline in movement in the OFT compared to control groups (FIG. 5B). After 8 weeks, mice were sacrificed for histological analysis to assess neuropathological changes associated with SAS3 treatment. The substantia nigra (SN) and observed aggregates in both SN pars compacta (SNpc) and reticulata (SNr) were first examined. Strikingly, the SNpc exhibited a clear reduction in TH+ neurons (FIG. 5C), and the intensity of DA neuron projections was significantly diminished in the striatum (FIGS. 5D-E). Next, the olfactory bulb (OB), which PHP.eB strongly transduces, was examined. Consistent with the in vitro findings, SAS-induced aggregates were associated with robust pS129-αSyn staining, whereas the control (hSyn-rtTA only), SNCA OE, and SAP treatments did not induce significant pS129-αSyn signal (FIG. 5F and FIG. 11A). Aggregates were also found in other brain regions such as cortex and hippocampus (FIG. 11B). Finally, proteinase K treatment of a SAS3-transduced brain confirmed the resistance of the aggregates to proteolytic degradation (FIG. 5G), further supporting their pathological relevance.

### Dopaminergic Neuron-Targeted SAS Recapitulates Progressive Motor Dysfunction and αSyn Aggregate Propagation in Mice

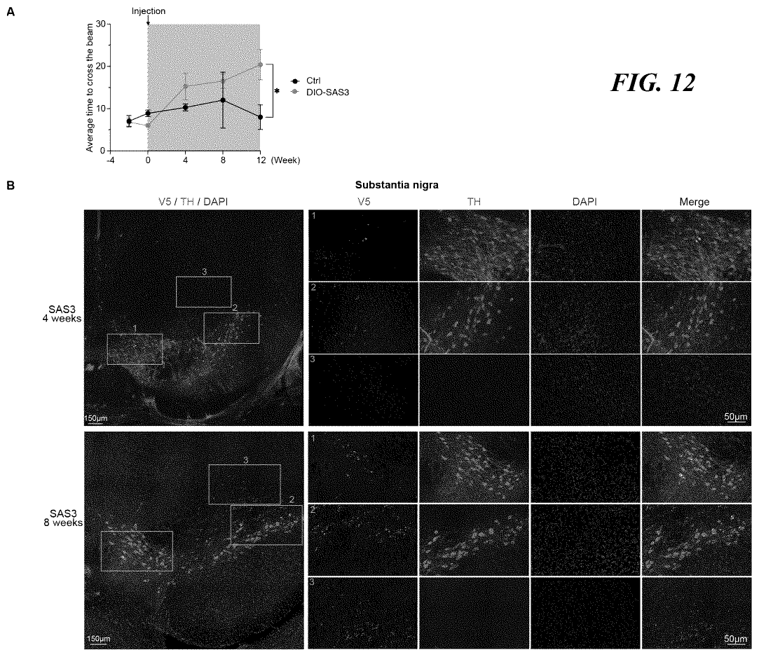

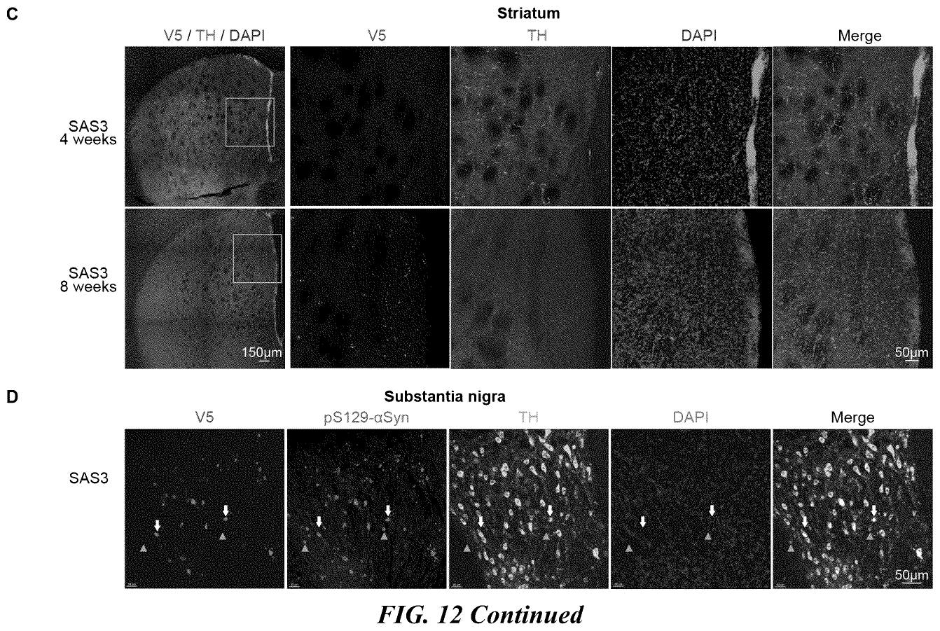

As a genetically encoded system, SAS provides a well-defined platform to dissect the roles of specific cell types in PD pathology. Degeneration of DA neurons in the SNpc causes motor symptoms in PD. To selectively deliver SAS3 into DA neurons, Cre-dependent SAS3 (DIO-SAS3) was injected into TH-Cre mice using AAV-PHP.eB (FIG. 511). 4 weeks post virus injection, SAS3-treated mice started to exhibit significant motor impairments in the beam-crossing assay, requiring more time to traverse the beam (FIG. 12, panel A) and displaying an increased number of slips (FIG. 5I) compared to control mice. The motor deficits progressively worsened with prolonged dox treatment, indicating ongoing neurodegeneration. Mice were sacrificed at 4, 8 and 12 weeks for histological analysis to assess the progression of pathology. DA neurons in the SN were first examined (FIG. 5J and FIG. 12, panel B). At week 4, SAS-induced aggregates were primarily detected in DA neurons within the ventral tegmental area (VTA) and SNpc. By week 8, the number of aggregates had increased, and many TH+ neurons in the SNpc exhibited a clear reduction in TH expression (FIG. 12, panel B). At week 12, while most neurons with aggregates in the VTA retained TH expression, a significant proportion of neurons in the SNpc with aggregates exhibited markedly reduced or absent TH expression (FIG. 5J). SAS-induced aggregates were also detected in regions that are not typically targeted by TH-Cre, suggesting potential propagation of aggregates beyond DA neurons. A similar trend was observed in the striatum, the primary target of DA neurons from the SNpc. At week 4, no aggregates were detectable in the striatum (FIG. 12, panel C). However, aggregates emerged by week 8 (FIG. 12, panel C) and became more prominent by week 12 (FIG. 5K), suggesting progressive aggregate accumulation and potential spreading along the nigrostriatal pathway. The intensity of TH staining in the striatum also decreased with time (FIG. 5K and FIG. 12, panel C), reflecting a reduction in dopaminergic innervation, consistent with the observed neurodegeneration in the SNpc. Immunostaining for V5 and pS129-αSyn revealed that not only were SAS-induced aggregates strongly phosphorylated (FIG. 12, panel D, arrows), but a significant amount of endogenous α-synuclein also aggregated and became phosphorylated (FIG. 12, panel D, arrowheads). This again confirmed that SAS3 can induce secondary nucleation of αSyn, contributing to the spread of disease. Ubiquitin and p62/SQSTM1 immunostaining confirmed the molecular complexity of the SAS-induced aggregates and their resemblance to LBs (FIGS. 5L and 5M).

Targeting SAS expression to DA neurons recapitulated multiple hallmarks of Parkinson's disease, from motor impairment and dopaminergic degeneration to αSyn propagation and LB-like inclusion formation. These results establish SAS as a mechanistically informative platform for probing selective vulnerability and intercellular aggregate dynamics in vivo.

### SAS Activates Microglia and Triggers Immune Response in Mice

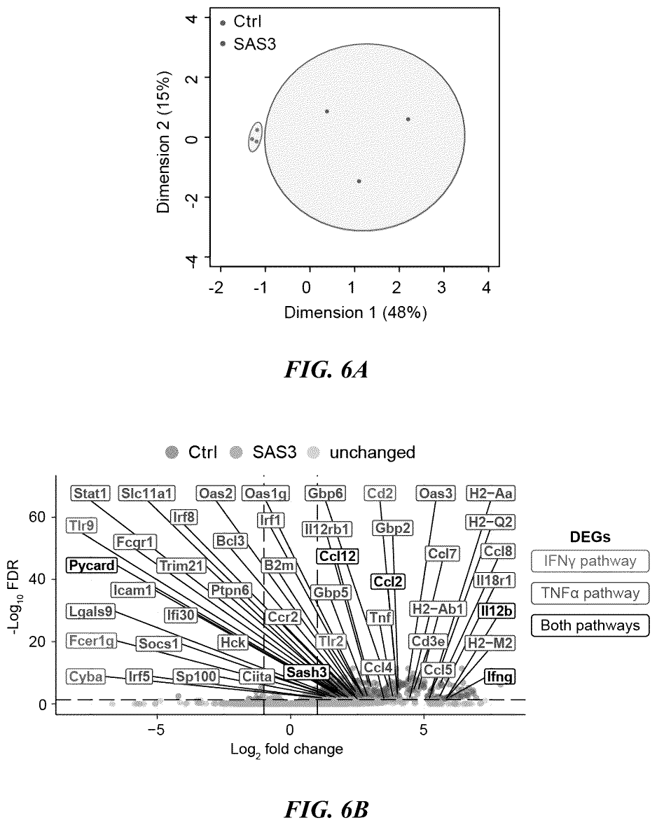

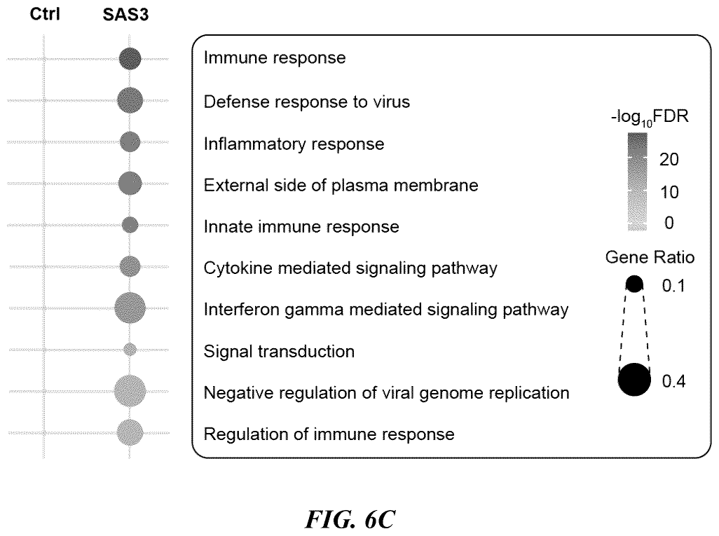

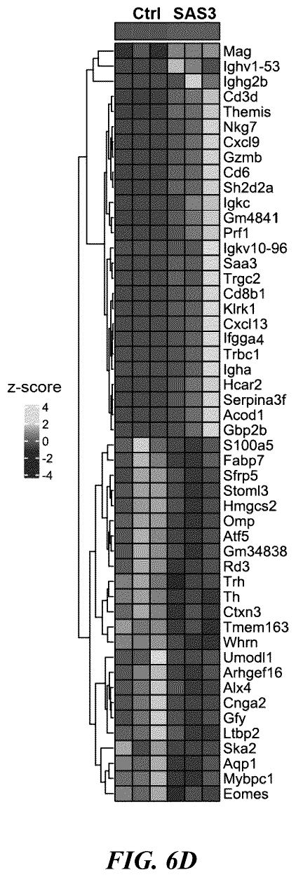

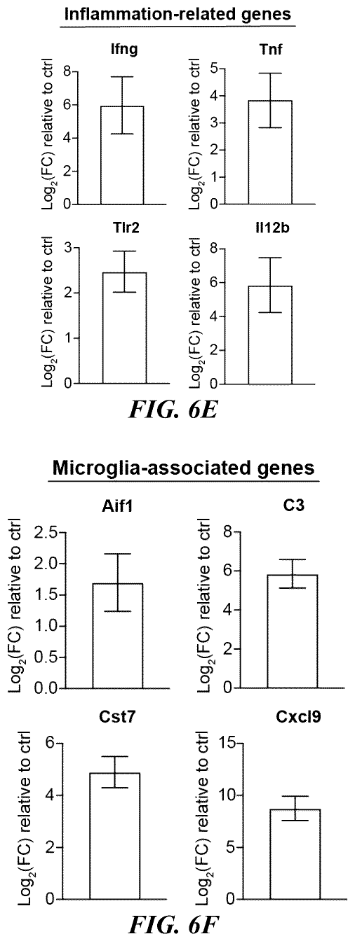

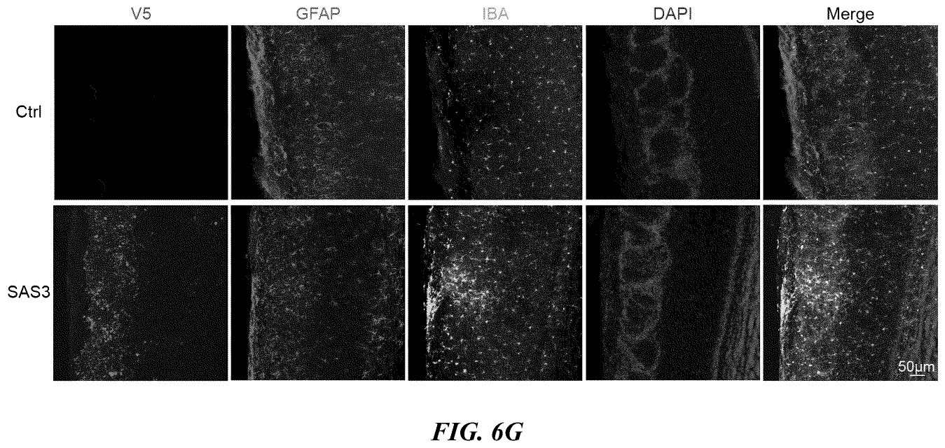

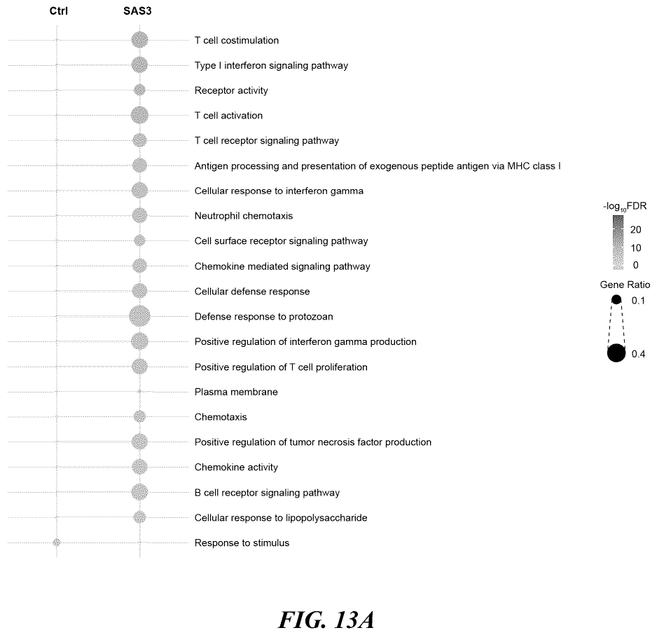

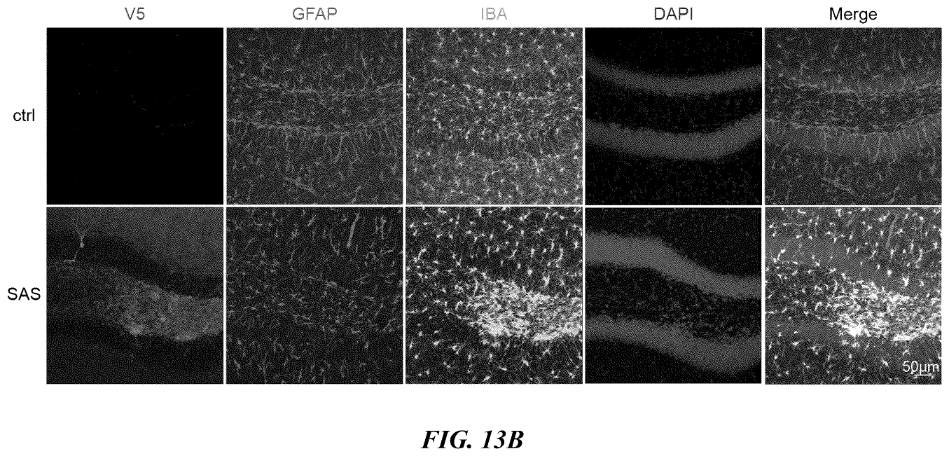

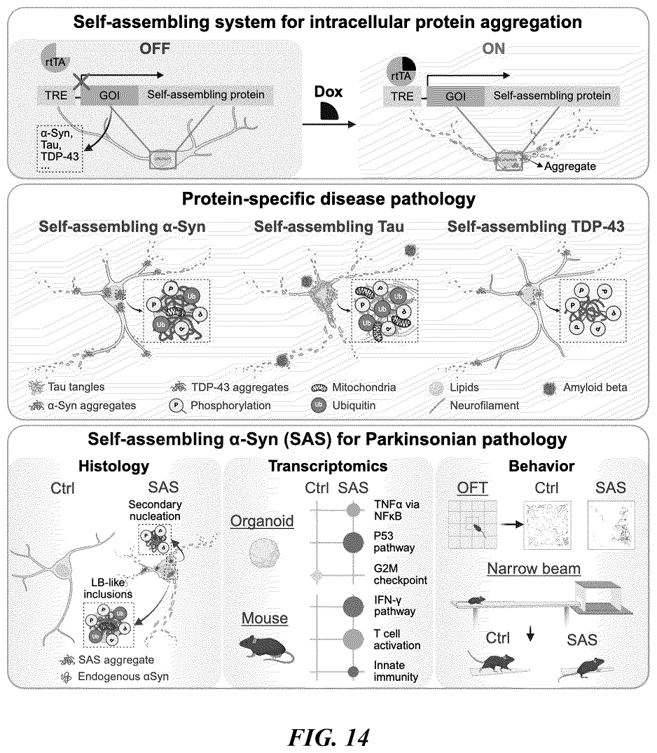

To dissect transcriptional changes in response to SAS-induced aggregates in vivo, bulk RNAseq of the OB, a region strongly transduced by PHP.eB and one of the earliest and consistently affected brain regions in PD, was performed. SAS-transduced mice displayed a distinct transcriptome compared with control mice expressing only rtTA (FIG. 6A). Differential gene expression analysis identified 139 DEGs with adjusted p-values <0.05 and |log 2FCl >1, with a strong enrichment of genes promoting inflammation (FIG. 6B). Pathway analysis confirmed robust activation of inflammatory and immune responses (FIG. 6C and FIG. 13A). Among the top 50 most dysregulated genes (FIG. 6D), several key inflammatory pathways were highly upregulated, including interferon-gamma (IFN-7), tumor necrosis factor-alpha (TNF-α), toll-like receptor (TLR), and interleukin (IL) signaling (FIG. 6E).

A significant upregulation of genes associated with microglial activation in SAS-transduced mice was also observed (FIG. 6F). For example, IBA1, encoded by the Aif1 gene, was markedly increased in SAS3-treated mice. CXCL9, a chemokine produced by microglia that plays a crucial role in neuroimmune responses, exhibited an approximately 500-fold upregulation. CST7, a cystatin that has been implicated in microglial activation under disease conditions and is linked to pathways involved in phagocytosis, cytokine/chemokine signaling, and antigen presentation, was also highly expressed. Additionally, complement protein C3, a key activator of microglia, showed a 60-fold increase. These changes strongly indicate a pronounced microglial activation state in SAS3-treated mice. Immunostaining of astrocytes (GFAP) and microglia (IBA1) confirmed that activated microglia clustered around SAS-induced aggregates, suggesting a targeted immune response to pathological protein accumulation (FIG. 6G and FIG. 13B). Collectively, these findings highlight the profound neuroinflammatory impact of SAS-induced aggregates, mirroring key aspects of PD pathology.

### DISCUSSION

The present disclosure provides a synthetic biology platform that enables inducible, tunable, and cell-type-specific formation of intracellular aggregates of disease-relevant proteins in diverse model systems. This platform faithfully recreates hallmark features of human proteinopathies in vitro and in vivo, including inclusion localization, morphology and composition, transcriptional responses, and behavioral phenotypes. Using self-assembling a-synuclein (SAS), a PD model was established which recapitulates Lewy body-like pathology, dopaminergic neuron loss, aggregate propagation along the nigrostriatal axis, and progressive motor deficits. Importantly, SAS-induced aggregates incorporate mitochondria, lipids, ubiquitin, and p62, molecular signatures of bona fide Lewy bodies that have been difficult to reproduce in other disease models. The system also revealed prion-like seeding of endogenous αSyn and elicited robust glial responses, offering a powerful framework to study cell-nonautonomous aspects of disease progression. Moreover, by extending this approach to Tau and TDP-43, the platform's generalizability across major neurodegenerative diseases was demonstrated. The observed differences in organelle interactions and toxicity of different protein aggregates illuminate potential mechanisms of selective vulnerability and disease divergence.

The initial SAS construct screening discovered that different types of αSyn aggregates exhibit widely varying capacities to induce pS129-αSyn, a key disease marker. This raises critical questions about what structural and biochemical features define a toxic aggregate. The present findings suggest that local concentration, structural conformation, and context-dependent modifications play a decisive role. For example, SAS3-induced aggregates exhibit significantly higher toxicity than SNCA OE despite a lower total amount of αSyn (FIGS. 1C-D and FIG. 7B-C). Similar observations have been made comparing αSyn PFFs and αSyn monomers. Structural differences appear to influence pathological outcomes, as different SAS constructs induce different levels of toxicity and phosphorylation (FIG. 7A). This aligns with studies showing that patient-derived αSyn PFFs exhibit different structural properties and varying neurotoxicity. These observations imply that the structural conformation of αSyn aggregates may be a key determinant of disease progression and severity. Deciphering the structural determinants of aggregate toxicity may be essential for understanding disease progression and designing targeted interventions.

A major strength of this platform is its broad applicability across in vitro and in vivo experimental systems, including cell lines (FIGS. 1A-J), primary neurons (FIG. 2), hESC or iPSC-derived neurons, organoids (FIGS. 3A-J and FIGS. 4A-H), and mice (FIGS. 5A-M and FIGS. 6A-G). Notably, in hESC-derived neurons and brain organoids, SAS, SA-Tau and SA-TDP enable robust recapitulation of PD, AD, and ALS hallmarks, such as LB formation, extracellular Aβ production, and TDP-43 cytoplasmic inclusions, which previous models have failed to reproduce. This provides a compelling reason to integrate the present self-assembling system with patient-derived iPSCs to dissect the impact of genetic variants and environmental factors on disease onset and progression. The system's tunability also makes it well suited for high-throughput screens to identify disease modifiers and therapeutic compounds.

The present disclosure provides direct evidence in primary neurons that SAS induces secondary nucleation of endogenous αSyn (FIGS. 4B and 4C). This finding is further supported by the presence of V5-negative, pS129-positive aggregates in neurons in multiple, in vitro and in vivo, systems (FIG. 2, panel B; FIG. 8; FIG. 9, panel A; FIG. 10, panel B; FIG. 12, panel D). Secondary nucleation is a critical driver of αSyn aggregation spread and amplification, yet its underlying mechanism remains unclear. Future studies are needed to dissect how primary nucleation triggers secondary nucleation and, more importantly, to identify strategies to halt secondary nucleation, which could be key to developing effective disease-modifying therapies for PD.

Neurodegenerative diseases often exhibit selective neuronal vulnerability, and single-cell RNA sequencing (scRNA-seq) studies have identified distinct neuronal subtypes that are more susceptible to degeneration in PD. This study successfully demonstrated the ability to deliver SAS into specific neuron types non-invasively using AAV-PHP.eB. By targeting different neuronal subpopulations using cell-type-specific Cre lines or promoters, one can systematically investigate the roles of various neuronal populations in driving diverse neurodegenerative pathologies. This approach provides a powerful strategy to dissect the contributions of specific neuronal populations to disease progression, offering insights into why certain neurons are particularly susceptible and identifying potential therapeutic targets to protect these vulnerable populations.

While the primary focus was PD, αSyn aggregation is also central to other synucleinopathies, such as dementia with Lewy bodies (DLB) and multiple system atrophy (MSA)⁹. The SAS system can be readily extended to study these disorders, investigating potentially distinct or overlapping pathological mechanisms. More broadly, the synthetic approach provides a modular, programmable framework for modeling protein aggregation across a wide range of diseases. By applying this strategy to αSyn, Tau and TDP-43, key features of PD, AD and ALS were successfully recapitulated, respectively. By enabling side-by-side comparison of different disease-associated aggregates in a unified system, this platform opens the door to comparative pathology studies and mechanistic discovery across the neurodegenerative disease spectrum. In addition, protein aggregation is increasingly recognized in non-neurological diseases. For example, islet amyloid polypeptide (IAPP) aggregation cause 3-cell dysfunction in type 2 diabetes, and aggregation of mutant p53 has been implicated in solid tumors. As such, this platform may hold value for a broad range of research communities beyond neuroscience.

Together, the present work introduces a scalable, versatile and highly disease-relevant strategy to model and dissect protein aggregation. This “build-to-understand” framework sets the stage for fundamental discoveries into the mechanisms of aggregation-driven pathology and for the development of effective, disease-modifying therapies.

TABLE 1
Exemplary proteopathic polypeptides

| | | SEQ |
|---|---|---|
| | | ID |
| | Amino Acid Sequence | NO |
| Human aSyn | MDVFMKGLSKAKEGVVAAAEKTKQGVAEAAGKTKEGVLYVGSKTKEGVVHGVATVAEKTKEQVTNVGGAVVTGV | 1 |
| | TAVAQKTVEGAGSIAAATGFVKKDQLGKNEEGAPQEGILEDMPVDPDNEAYEMPSEEGYQDYEPEA | |
| human aSyn | MDVFMKGLSKAKEGVVAAAEKTKQGVAEAAGKTKEGVLYVGSKTKEGVVHGVTTVAEKTKEQVTNVGGAVVTGV | 2 |
| (A53T) | TAVAQKTVEGAGSIAAATGFVKKDQLGKNEEGAPQEGILEDMPVDPDNEAYEMPSEEGYQDYEPEA | |
| marmoset | MDVFMKGLSKAKEGVVAAAEKTKQGVAEAAGKTKEGVLYVGSKTKEGVVHGVTTVAEKTKEQVTNVGGAVVTGV | 3 |
| aSyn | TAVAQKTVEGAGNIAAATGFVKKDHLGKSEEGAPQEGILEDMPVDPDNEAYEMPSEEGYQDYEPEA | |
| mouse aSyn | MDVFMKGLSKAKEGVVAAAEKTKQGVAEAAGKTKEGVLYVGSKTKEGVVHGVTTVAEKTKEQVTNVGGAVVTGV | 4 |
| | TAVAQKTVEGAGNIAAATGFVKKDQMGKGEEGYPQEGILEDMPVDPGSEAYEMPSEEGYQDYEPEA | |
| Human Tau | MAEPRQEFEVMEDHAGTYGLGDRKDQGGYTMHQDQEGDTDAGLKESPLQTPTEDGSEEPGSETSDAKSTPTAEDVTA | 5 |
| 2N4R | PLVDEGAPGKQAAAQPHTEIPEGTTAEEAGIGDTPSLEDEAAGHVTQARMVSKSKDGTGSDDKKAKGADGKTKIATPR | |
| | GAAPPGQKGQANATRIPAKTPPAPKTPPSSGEPPKSGDRSGYSSPGSPGTPGSRSRTPSLPTPPTREPKKVAVVRTPP | |
| | KSPSSAKSRLQTAPVPMPDLKNVKSKIGSTENLKHQPGGGKVQIINKKLDLSNVQSKCGSKDNIKHVPGGGSVQIVYK | |
| | PVDLSKVTSKCGSLGNIHHKPGGGQVEVKSEKLDFKDRVQSKIGSLDNITHVPGGGNKKIETHKLTFRENAKAKTDHG | |
| | AEIVYKSPVVSGDTSPRHLSNVSSTGSIDMVDSPQLATLADEVSASLAKQGL | |
| human Tau | MAEPRQEFEVMEDHAGTYGLGDRKDQGGYTMHQDQEGDTDAGLKESPLQTPTEDGSEEPGSETSDAKSTPTAEAEEA | 6 |
| IN4R | GIGDTPSLEDEAAGHVTQARMVSKSKDGTGSDDKKAKGADGKTKIATPRGAAPPGQKGQANATRIPAKTPPAPKTPPSS | |
| | GEPPKSGDRSGYSSPGSPGTPGSRSRTPSLPTPPTREPKKVAVVRTPPKSPSSAKSRLQTAPVPMPDLKNVKSKIGSTE | |
| | NLKHQPGGGKVQIINKKLDLSNVQSKCGSKDNIKHVPGGGSVQIVYKPVDLSKVTSKCGSLGNIHHKPGGGQVEVKSEK | |
| | LDFKDRVQSKIGSLDNITHVPGGGNKKIETHKLTFRENAKAKTDHGAEIVYKSPVVSGDTSPRHLSNVSSTGSIDMVDSP | |
| | QLATLADEVSASLAKQGL | |
| human Tau | MAEPRQEFEVMEDHAGTYGLGDRKDQGGYTMHQDQEGDTDAGLKESPLQTPTEDGSEEPGSETSDAKSTPTAEDVTA | 7 |
| 2N3R | PLVDEGAPGKQAAAQPHTEIPEGTTAEEAGIGDTPSLEDEAAGHVTQARMVSKSKDGTGSDDKKAKGADGKTKIATPR | |
| | GAAPPGQKGQANATRIPAKTPPAPKTPPSSGEPPKSGDRSGYSSPGSPGTPGSRSRTPSLPTPPTREPKKVAVVRTPP | |
| | KSPSSAKSRLQTAPVPMPDLKNVKSKIGSTENLKHQPGGGKVQIVYKPVDLSKVTSKCGSLGNIHHKPGGGQVEVKSEK | |
| | LDFKDRVQSKIGSLDNITHVPGGGNKKIETHKLTFRENAKAKTDHGAEIVYKSPVVSGDTSPRHLSNVSSTGSIDMVDS | |
| | PQLATLADEVSASLAKQGL | |
| human Tau | MAEPRQEFEVMEDHAGTYGLGDRKDQGGYTMHQDQEGDTDAGLKESPLQTPTEDGSEEPGSETSDAKSTPTAEAEEA | 8 |
| IN3R | GIGDTPSLEDEAAGHVTQARMVSKSKDGTGSDDKKAKGADGKTKIATPRGAAPPGQKGQANATRIPAKTPPAPKTPPS | |
| | SGEPPKSGDRSGYSSPGSPGTPGSRSRTPSLPTPPTREPKKVAVVRTPPKSPSSAKSRLQTAPVPMPDLKNVKSKIGST | |
| | ENLKHQPGGGKVQIVYKPVDLSKVTSKCGSLGNIHHKPGGGQVEVKSEKLDFKDRVQSKIGSLDNITHVPGGGNKKIET | |
| | HKLTFRENAKAKTDHGAEIVYKSPVVSGDTSPRHLSNVSSTGSIDMVDSPQLATLADEVSASLAKQGL | |
| human Tau | MAEPRQEFEVMEDHAGTYGLGDRKDQGGYTMHQDQEGDTDAGLKAEEAGIGDTPSLEDEAAGHVTQARMVSKSKDGT | 9 |
| ON4R | GSDDKKAKGADGKTKIATPRGAAPPGQKGQANATRIPAKTPPAPKTPPSSGEPPKSGDRSGYSSPGSPGTPGSRSRTP | |
| | SLPTPPTREPKKVAVVRTPPKSPSSAKSRLQTAPVPMPDLKNVKSKIGSTENLKHQPGGGKVQIINKKLDLSNVQSKC | |
| | GSKDNIKHVPGGGSVQIVYKPVDLSKVTSKCGSLGNIHHKPGGGQVEVKSEKLDFKDRVQSKIGSLDNITHVPGGGNK | |
| | KIETHKLTFRENAKAKTDHGAEIVYKSPVVSGDTSPRHLSNVSSTGSIDMVDSPQLATLADEVSASLAKQGL | |
| human Tau | MAEPRQEFEVMEDHAGTYGLGDRKDQGGYTMHQDQEGDTDAGLKAEEAGIGDTPSLEDEAAGHVTQARMVSKSKD | 10 |
| 0N3R | GTGSDDKKAKGADGKTKIATPRGAAPPGQKGQANATRIPAKTPPAPKTPPSSGEPPKSGDRSGYSSPGSPGTPGSRSRTP | |
| | SLPTPPTREPKKVAVVRTPPKSPSSAKSRLQTAPVPMPDLKNVKSKIGSTENLKHQPGGGKVQIVYKPVDLSKVTSKCG | |
| | SLGNIHHKPGGGQVEVKSEKLDFKDRVQSKIGSLDNITHVPGGGNKKIETHKLTFRENAKAKTDHGAEIVYKSPVVSGD | |
| | TSPRHLSNVSSTGSIDMVDSPQLATLADEVSASLAKQGL | |
| mouse Tau | MADPRQEFDTMEDHAGDYTLLQDQEGDMDHGLKESPPQPPADDGAEEPGSETSDAKSTPTAEDVTAPLVDERAPDKQA | 11 |
| 2N4R | AAQPHTEIPEGITAEEAGIGDTPNQEDQAAGHVTQARVASKDRTGNDEKKAKGADGKTGAKIATPRGAASPAQKGTSNA | |
| | TRIPAKTTPSPKTPPGSGEPPKSGERSGYSSPGSPGTPGSRSRTPSLPTPPTREPKKVAVVRTPPKSPSASKSRLQTAPV | |
| | PMPDLKNVRSKIGSTENLKHQPGGGKVQIINKKLDLSNVQSKCGSKDNIKHVPGGGSVQIVYKPVDLSKVTSKCGSLGN | |
| | IHHKPGGGQVEVKSEKLDFKDRVQSKIGSLDNITHVPGGGNKKIETHKLTFRENAKAKTDHGAEIVYKSPVVSGDTSPR | |
| | HLSNVSSTGSIDMVDSPQLATLADEVSASLAKQGL | |
| human TDP43 | MSEYIRVTEDENDEPIEIPSEDDGTVLLSTVTAQFPGACGLRYRNPVSQCMRGVRLVEGILHAPDAGWGNLVYVVNYP | 12 |
| | KDNKRKMDETDASSAVKVKRAVQKTSDLIVLGLPWKTTEQDLKEYFSTFGEVLMVQVKKDLKTGHSKGFGFVRFTEY | |
| | ETQVKVMSQRHMIDGRWCDCKLPNSKQSQDEPLRSRKVFVGRCTEDMTEDELREFFSQYGDVMDVFIPKPFRAFAFVT | |
| | FADDQIAQSLCGEDLIIKGISVHISNAEPKHNSNRQLERSGRFGGNPGGFGNQGGFGNSRGGGAGLGNNQGSNMGGGM | |
| | NFGAFSINPAMMAAAQAALQSSWGMMGMLASQQNQSGPSGNNQNQGNMQREPNQAFGSGNNSYSGSNSGAAIGWG | |
| | SASNAGSGSGFNGGFGSSMDSKSSGWGM | |
| mouse TDP43 | MSEYIRVTEDENDEPIEIPSEDDGTVLLSTVTAQFPGACGLRYRNPVSQCMRGVRLVEGILHAPDAGWGNLVYVVNYP | 13 |
| | KDNKRKMDETDASSAVKVKRAVQKTSDLIVLGLPWKTTEQDLKDYFSTFGEVLMVQVKKDLKTGHSKGFGFVRFTE | |
| | YETQVKVMSQRHMIDGRWCDCKLPNSKQSPDEPLRSRKVFVGRCTEDMTAEELQQFFCQYGEVVDVFIPKPFRAFAFV | |
| | TFADDKVAQSLCGEDLIIKGISVHISNAEPKHNSNRQLERSGREGGNPGGFGNQGGFGNSRGGGAGLGNNQGGNMGGG | |
| | MNFGAFSINPAMMAAAQAALQSSWGMMGMLASQQNQSGPSGNNQSQGSMQREPNQAFGSGNNSYSGSNSGAPLGW | |
| | GSASNAGSGSGFNGGFGSSMDSKSSGWGM | |

TABLE 2
Exemplary self-assembling domains

| | Amino Acid Sequence | SEQ ID NO |
|---|---|---|
| 1POK | MIDYTAAGFTLLQGAHLYAPEDRGICDVLVANGKIIAVASNIPSDIVPNCTVVDLSGQILCPGFIDQHVHLIGGGGEAGPTTRT | 14 |
| (E239Y) | PEVALSRLTEAGVTSVVGLLGTDSISRHPESLLAKTRALNEEGISAWMLTGAYHVPSRTITGSVEKDVAIIDRVIGVKCAISDH | |
| | RSAAPDVYHLANMAAESRVGGLLGGKPGVTVFHMGDSKKALQPIYDLLENCDVPISKLLPTHVNRNVPLFYQALEFARKG | |
| | GTIDITSSIDEPVAPAEGIARAVQAGIPLARVTLSSDGNGSQPFFDDEGNLTHIGVAGFETLLETVQVLVKDYDFSISDALRPLT | |
| | SSVAGFLNLTGKGEILPGNDADLLVMTPELRIEQVYARGKLMVKDGKACVKGTFETAGGSGGTGGSGGTGGSGGTGGSGG | |
| | TGGKPIPNPLLGLDSTGSGKIEEGKLVIWINGDKGYNGLAEVGKKFEKDTGIKVTVEHPDKLEEKFPQVAATGDGPDIIFWAH | |
| | DRFGGYAQSGLLAEITPDKAFQDKLYPFTWDAVRYNGKLIAYPIAVEALSLIYNKDLLPNPPKTWEEIPALDKELKAKGKSA | |
| | LMFNLQEPYFTWPLIAADGGYAFKYENGKYDIKDVGVDNAGAKAGLTFLVDLIKNKHMNADTDYSIAEAAFNKGETAMTI | |
| | NGPWAWSNIDTSKVNYGVTVLPTFKGQPSKPFVGVLSAGINAASPNKELAKEFLENYLLTDEGLEAVNKDKPLGAVALKSY | |
| | EEELAKDPRIAATMENAQKGEIMPNIPQMSAFWYAVRTAVINAASGRQTVDEALKDAQT | |
| DHF40 | MSSEKEELRERLVKICVELAKLKGDDTLKAAEAAEEAFRLVVLAAMLAGIDSSEVLELAIRLIKTCVVLAAMEGYDISEACR | 15 |
| | AAAEAFTRVAMAALRAGITSSLVLKAAIELIKECVLNAAVEGYDISEACRAAAEAFKRVAEAAKRAGITSLETLLRAIEEIRK | |
| | RVEEAQREGNDISEACRQAAEEFRKKAEELKRRGDV | |
| DHF46 | TKEERVLLMKVAILAIVAAKKGNTDEVRKALELALLIAKVSGTTEAVKLALEVVARVAIEAARRGNTDAVREALEVALEIA | 16 |
| | RESGTTEAVKLALEVVARVAIEAARRGNTEAVVEALLVALEIAKESGTEEAVRLALEVVKRVSNEALKQGNVDAVKVALE | |
| | VRKMIEELS | |
| 2VYC | MKVLIVESEFLHQDTWVGNAVERLADALSQQNVTVIKSTSFDDGFAILSSNEAIDCLMFSYQMEHPDEHQNVRQLIGKLHER | 17 |
| | QQNVPVFLLGDREKALAAMDRDLLELVDEFAWILEDTADFIAGRAVAAMTRYRQQLLPPLFSALMKYSDIHEYSWAAPGH | |
| | QGGVGFTKTPAGRFYHDYYGENLFRTDMGIERTSLGSLLDHTGAFGESEKYAARVFGADRSWSVVVGTSGSNRTIMQACM | |
| | TDNDVVVVDRNCHKSIEQGLMLTGAKPVYMVPSRNRYGIIGPIYPQEMQPETLQKKISESPLTKDKAGQKPSYCVVTNCTY | |
| | DGVCYNAKEAQDLLEKTSDRLHFDEAWYGYARFNPIYADHYAMRGEPGDHNGPTVFATHSTHKLLNALSQASYIHVREGR | |
| | GAINFSRFNQAYMMHATTSPLYAICASNDVAVSMMDGNSGLSLTQEVIDEAVDFRQAMARLYKEFTADGSWFFKPWNKE | |
| | VVTDPQTGLTYLFALAPKLLTTVQDCWVMHPGESWHGFKDIPDNWSMLDPIKVSILAPGMGEDGELEETGVPAALVTAWL | |
| | GRHGIVPTRTTDFQIMFLFSMGVTRGKWGTLVNTLCSFKRHYDANTPLAQVMPELVEQYPDTYANMGIHDLGDTMFAWLK | |
| | ENNPGARLNEAYSGLPVAEVTPREAYNAIVDNNVELVSIENLPGRIAANSVIPYPPGIPMLLSGENFGDKNSPQVSYLRSLQS | |
| | WDHHFPGFEHETEGTEIIDGIYHVMCVKAGGSGGT | |
| γPFD | MVNEVIDINEAVRAYIAQIEGLRAEIGRLDATIATLRQSLATLKSLKTLGEGKTVLVPVGSIAQVEMKVEKMDKVVVSVGQN | 18 |
| | ISAELEYEEALKYIEDEIKKLLTFRLVLEQAIAELYAKIEDLIAEAQQTSEEEKAEEEENEEKAE | |
| β-tubulin | MGDVGPRWANWDPSRSGVRDHPGQQSETLPHATFLQKIKFWEVISDEHGIDPTGTYHGDSDLQLDRISVYYNEATGGKYVP | 19 |
| | RAILVDLEPGTMDSVRSGPFGQIFRPDNFVFGQSGAGNNWAKGHYTEGAELVDSVLDVVWKEAESCDCLQGFQLTHSLGGG | |
| | TGSGMGTLLISKIREEYPDRIMNTFSVVPSPKVSDTVVEPYNATLSVHQLVENTDETYCIDNEALYDICFRTLKLTTPTYGDL | |
| | NHLVSATMSGVTTCLRFPGQLNADLRKLAVNMVPFPRLHFFMPGFAPLTSRGSQQYRALTVPELTQQVFDAKNMMAACDP | |
| | RHGRYLTVAAVFRGRMSMKEVDEQMLNVQNKNSSYFVEWIPNNVKTAVCDIPPRGLKMAVTFIGNSTAIQELFKRISEQFT | |
| | AMFRRKAFLHWYTGEGMDEMEFTEAESNMNDLVSEYQQYQDATAEEEEDFGEEAEEEA | |
| α-actin | MDDDIAALVVDNGSGMCKAGFAGDDAPRAVFPSIVGRPRHQGVMVGMGQKDSYVGDEAQSKRGILTLKYPIEHGIVTNW | 20 |
| | DDMEKIWHHTFYNELRVAPEEHPVLLTEAPLNPKANREKMTQIMFETFNTPAMYVAIQAVLSLYASGRTTGIVMDSGDGVT | |
| | HTVPIYEGYALPHAILRLDLAGRDLTDYLMKILTERGYSFTTTAEREIVRDIKEKLCYVALDFEQEMATAASSSSLEKSYELP | |
| | DGQVITIGNERFRCPEALFQPSFLGMESCGIHETTFNSIMKCDVDIRKDLYANTVLSGGTTMYPGIADRMQKEITALAPSTMKI | |
| | KIIAPPERKYSVWIGGSILASLSTFQQMWISKQEYDESGPSIVHRKCF | |
| Tom5 | MFRIEGLAPKLDPEEMKRKMREDVISSIRNFLIYVALLRVTPFILKKLDSI | 21 |
| Tom20 | MVGRNSAIAAGVCGALFIGYCIYF | 22 |

TABLE 3
Exemplary sequences of self-assembling aSyn fusion protein

| | | Source of | | SEQ |
|---|---|---|---|---|
| | Construct | SAP | | ID |
| Code | design | sequence | Amino Acid Sequence | NO |
| SNCA OE | aSyn- | N/A | MDVFMKGLSKAKEGVVAAAEKTKQGVAEAAGKTKEGVLYVGSKTKEGVVHGVATVAEKTKEQVTNVGGAVV | 23 |
| | Linker-V5 | | TGVTAVAQKTVEGAGSIAAATGFVKKDQLGKNEEGAPQEGILEDMPVDPDNEAYEMPSEEGYQDYEPEAGGGSG | |
| | | | GTGGGKPIPNPLLGLDST | |
| SAS3 | aSyn- | Garcia- | MDVFMKGLSKAKEGVVAAAEKTKQGVAEAAGKTKEGVLYVGSKTKEGVVHGVATVAEKTKEQVTNVGGAVV | 24 |
| | Linker-V5- | Seisdedos | TGVTAVAQKTVEGAGSIAAATGFVKKDQLGKNEEGAPQEGILEDMPVDPDNEAYEMPSEEGYQDYEPEAGGSGG | |
| | 1POK(E239 | et al., | TGGMIDYTAAGFTLLQGAHLYAPEDRGICDVLVANGKIIAVASNIPSDIVPNCTVVDLSGQILCPGFIDQHVHLIGG | |
| | Y)-Linker- | 2017 | GGEAGPTTRTPEVALSRLTEAGVTSVVGLLGTDSISRHPESLLAKTRALNEEGISAWMLTGAYHVPSRTITGSVEK | |
| | MBP | | DVAIIDRVIGVKCAISDHRSAAPDVYHLANMAAESRVGGLLGGKPGVTVFHMGDSKKALQPIYDLLENCDVPISK | |
| | | | LLPTHVNRNVPLFYQALEFARKGGTIDITSSIDEPVAPAEGIARAVQAGIPLARVTLSSDGNGSQPFFDDEGNLTHIG | |
| | | | VAGFETLLETVQVLVKDYDFSISDALRPLTSSVAGFLNLTGKGEILPGNDADLLVMTPELRIEQVYARGKLMVKD | |
| | | | GKACVKGTFETAGGSGGTGGSGGTGGSGGTGGSGGTGGKPIPNPLLGLDSTGSGKIEEGKLVIWINGDKGYNGLA | |
| | | | EVGKKFEKDTGIKVTVEHPDKLEEKFPQVAATGDGPDIIFWAHDRFGGYAQSGLLAEITPDKAFQDKLYPFTWDA | |
| | | | VRYNGKLIAYPIAVEALSLIYNKDLLPNPPKTWEEIPALDKELKAKGKSALMFNLQEPYFTWPLIAADGGYAFKYE | |
| | | | NGKYDIKDVGVDNAGAKAGLTFLVDLIKNKHMNADTDYSIAEAAFNKGETAMTINGPWAWSNIDTSKVNYGVT | |
| | | | VLPTFKGQPSKPFVGVLSAGINAASPNKELAKEFLENYLLTDEGLEAVNKDKPLGAVALKSYEEELAKDPRIAAT | |
| | | | MENAQKGEIMPNIPQMSAFWYAVRTAVINAASGRQTVDEALKDAQT | |
| SAS4 | aSyn- | Shen et | MDVFMKGLSKAKEGVVAAAEKTKQGVAEAAGKTKEGVLYVGSKTKEGVVHGVATVAEKTKEQVTNVGGAVV | 25 |
| | Linker- | al., 2018 | TGVTAVAQKTVEGAGSIAAATGFVKKDQLGKNEEGAPQEGILEDMPVDPDNEAYEMPSEEGYQDYEPEAGGSGG | |
| | DHF40- | | TGGMSSEKEELRERLVKICVELAKLKGDDTLKAAEAAEEAFRLVVLAAMLAGIDSSEVLELAIRLIKTCVVLAAM | |
| | Linker-V5 | | EGYDISEACRAAAEAFTRVAMAALRAGITSSLVLKAAIELIKECVLNAAVEGYDISEACRAAAEAFKRVAEAAKR | |
| | | | AGITSLETLLRAIEEIRKRVEEAQREGNDISEACRQAAEEFRKKAEELKRRGDVGGGSGGTGGGKPIPNPLLGLDST | |
| SAS5 | aSyn- | Shen et | MDVFMKGLSKAKEGVVAAAEKTKQGVAEAAGKTKEGVLYVGSKTKEGVVHGVATVAEKTKEQVTNVGGAVV | 26 |
| | Linker- | al., 2018 | TGVTAVAQKTVEGAGSIAAATGFVKKDQLGKNEEGAPQEGILEDMPVDPDNEAYEMPSEEGYQDYEPEAGGSGG | |
| | DHF46- | | TGGTKEERVLLMKVAILAIVAAKKGNTDEVRKALELALLIAKVSGTTEAVKLALEVVARVAIEAARRGNTDAVR | |
| | Linker-V5 | | EALEVALEIARESGTTEAVKLALEVVARVAIEAARRGNTEAVVEALLVALEIAKESGTEEAVRLALEVVKRVSNE | |
| | | | ALKQGNVDAVKVALEVRKMIEELSGGGSGGTGGGKPIPNPLLGLDST | |
| SAS6 | 2VYC- | Garcia- | MKVLIVESEFLHQDTWVGNAVERLADALSQQNVTVIKSTSFDDGFAILSSNEAIDCLMFSYQMEHPDEHQNVRQL | 27 |
| | Linker-V5- | Seisdedos | IGKLHERQQNVPVFLLGDREKALAAMDRDLLELVDEFAWILEDTADFIAGRAVAAMTRYRQQLLPPLFSALMKY | |
| | linker-aSyn | et al., | SDIHEYSWAAPGHQGGVGFTKTPAGRFYHDYYGENLFRTDMGIERTSLGSLLDHTGAFGESEKYAARVFGADRS | |
| | | 2017 | WSVVVGTSGSNRTIMQACMTDNDVVVVDRNCHKSIEQGLMLTGAKPVYMVPSRNRYGIIGPIYPQEMQPETLQK | |
| | | | KISESPLTKDKAGQKPSYCVVTNCTYDGVCYNAKEAQDLLEKTSDRLHFDEAWYGYARFNPIYADHYAMRGEPG | |
| | | | DHNGPTVFATHSTHKLLNALSQASYIHVREGRGAINFSRFNQAYMMHATTSPLYAICASNDVAVSMMDGNSGLS | |
| | | | LTQEVIDEAVDFRQAMARLYKEFTADGSWFFKPWNKEVVTDPQTGLTYLFALAPKLLTTVQDCWVMHPGESWH | |
| | | | GFKDIPDNWSMLDPIKVSILAPGMGEDGELEETGVPAALVTAWLGRHGIVPTRTTDFQIMFLFSMGVTRGKWGTL | |
| | | | VNTLCSFKRHYDANTPLAQVMPELVEQYPDTYANMGIHDLGDTMFAWLKENNPGARLNEAYSGLPVAEVTPRE | |
| | | | AYNAIVDNNVELVSIENLPGRIAANSVIPYPPGIPMLLSGENFGDKNSPQVSYLRSLQSWDHHFPGFEHETEGTEIID | |
| | | | GIYHVMCVKAGGSGGTGGSGGTGGGKPIPNPLLGLDSTGGSGGTGGMDVFMKGLSKAKEGVVAAAEKTKQGVA | |
| | | | EAAGKTKEGVLYVGSKTKEGVVHGVATVAEKTKEQVTNVGGAVVTGVTAVAQKTVEGAGSIAAATGFVKKDQ | |
| | | | LGKNEEGAPQEGILEDMPVDPDNEAYEMPSEEGYQDYEPEA | |
| SAS10 | yPFD- | Chen etal., | MVNEVIDINEAVRAYIAQIEGLRAEIGRLDATIATLRQSLATLKSLKTLGEGKTVLVPVGSIAQVEMKVEKMDKVV | 28 |
| | Linker-V5- | 2020 | VSVGQNISAELEYEEALKYIEDEIKKLLTFRLVLEQAIAELYAKIEDLIAEAQQTSEEEKAEEEENEEKAEGGSGGTG | |
| | Linker-aSyn | | GGKPIPNPLLGLDSTGGSGGTGGMDVFMKGLSKAKEGVVAAAEKTKQGVAEAAGKTKEGVLYVGSKTKEGVVH | |
| | | | GVATVAEKTKEQVTNVGGAVVTGVTAVAQKTVEGAGSIAAATGFVKKDQLGKNEEGAPQEGILEDMPVDPDNE | |
| | | | AYEMPSEEGYQDYEPEA | |
| SAS21 | aSyn- | Uniprot | MDVFMKGLSKAKEGVVAAAEKTKQGVAEAAGKTKEGVLYVGSKTKEGVVHGVATVAEKTKEQVTNVGGAVV | 29 |
| | Linker- | B4DY90 | TGVTAVAQKTVEGAGSIAAATGFVKKDQLGKNEEGAPQEGILEDMPVDPDNEAYEMPSEEGYQDYEPEAGGSGG | |
| | bTubulin- | | TGGMGDVGPRWANWDPSRSGVRDHPGQQSETLPHATFLQKIKFWEVISDEHGIDPTGTYHGDSDLQLDRISVYY | |
| | Linker-V5 | | NEATGGKYVPRAILVDLEPGTMDSVRSGPFGQIFRPDNFVFGQSGAGNNWAKGHYTEGAELVDSVLDVVWKEAE | |
| | | | SCDCLQGFQLTHSLGGGTGSGMGTLLISKIREEYPDRIMNTFSVVPSPKVSDTVVEPYNATLSVHQLVENTDETYCI | |
| | | | DNEALYDICFRTLKLTTPTYGDLNHLVSATMSGVTTCLRFPGQLNADLRKLAVNMVPFPRLHFFMPGFAPLTSRG | |
| | | | SQQYRALTVPELTQQVFDAKNMMAACDPRHGRYLTVAAVFRGRMSMKEVDEQMLNVQNKNSSYFVEWIPNNV | |
| | | | KTAVCDIPPRGLKMAVTFIGNSTAIQELFKRISEQFTAMFRRKAFLHWYTGEGMDEMEFTEAESNMNDLVSEYQQ | |
| | | | YQDATAEEEEDFGEEAEEEAGGSGGTGGGKPIPNPLLGLDST | |
| SAS22 | aSyn- | Uniprot | MDVFMKGLSKAKEGVVAAAEKTKQGVAEAAGKTKEGVLYVGSKTKEGVVHGVATVAEKTKEQVTNVGGAVV | 30 |
| | Linker- | P60709 | TGVTAVAQKTVEGAGSIAAATGFVKKDQLGKNEEGAPQEGILEDMPVDPDNEAYEMPSEEGYQDYEPEAGGSGG | |
| | bActin- | | TGGMDDDIAALVVDNGSGMCKAGFAGDDAPRAVFPSIVGRPRHQGVMVGMGQKDSYVGDEAQSKRGILTLKYP | |
| | Linker-V5 | | IEHGIVTNWDDMEKIWHHTFYNELRVAPEEHPVLLTEAPLNPKANREKMTQIMFETFNTPAMYVAIQAVLSLYAS | |
| | | | GRTTGIVMDSGDGVTHTVPIYEGYALPHAILRLDLAGRDLTDYLMKILTERGYSFTTTAEREIVRDIKEKLCYVAL | |
| | | | DFEQEMATAASSSSLEKSYELPDGQVITIGNERFRCPEALFQPSFLGMESCGIHETTFNSIMKCDVDIRKDLYANTV | |
| | | | LSGGTTMYPGIADRMQKEITALAPSTMKIKIIAPPERKYSVWIGGSILASLSTFQQMWISKQEYDESGPSIVHRKCFG | |
| | | | GSGGTGGGKPIPNPLLGLDST | |
| SAS25 | aSyn- | Uniprot | MDVFMKGLSKAKEGVVAAAEKTKQGVAEAAGKTKEGVLYVGSKTKEGVVHGVATVAEKTKEQVTNVGGAVV | 31 |
| | Linker- | Q8N4H5 | TGVTAVAQKTVEGAGSIAAATGFVKKDQLGKNEEGAPQEGILEDMPVDPDNEAYEMPSEEGYQDYEPEAGGSGG | |
| | Tom5- | | TGGMFRIEGLAPKLDPEEMKRKMREDVISSIRNFLIYVALLRVTPFILKKLDSIGGSGGTGGGKPIPNPLLGLDST | |
| | Linker-V5 | | | |
| SAS26 | Tom20- | Uniprot | MVGRNSAIAAGVCGALFIGYCIYFGGSGGTGGMDVFMKGLSKAKEGVVAAAEKTKQGVAEAAGKTKEGVLYV | 32 |
| | Linker- | Q15388 | GSKTKEGVVHGVATVAEKTKEQVTNVGGAVVTGVTAVAQKTVEGAGSIAAATGFVKKDQLGKNEEGAPQEGIL | |
| | aSyn- | | EDMPVDPDNEAYEMPSEEGYQDYEPEAGGSGGTGGGKPIPNPLLGLDST | |
| | Linker-V5 | | | |

TABLE 4
Exemplary sequences of self-assembling Tau fusion protein

| | | Source | | SEQ |
|---|---|---|---|---|
| | Construct | of SAP | | ID |
| Code | design | sequence | Amino Acid Sequence | NO |
| Tau OE | Tau- | N/A | MAEPRQEFEVMEDHAGTYGLGDRKDQGGYTMHQDQEGDTDAGLKESPLQTPTEDGSEEPGSETSDAKSTPTAEDVT | 33 |
| | Linker-V5 | | APLVDEGAPGKQAAAQPHTEIPEGTTAEEAGIGDTPSLEDEAAGHVTQARMVSKSKDGTGSDDKKAKGADGKTKIA | |
| | | | TPRGAAPPGQKGQANATRIPAKTPPAPKTPPSSGEPPKSGDRSGYSSPGSPGTPGSRSRTPSLPTPPTREPKKVAVVRTP | |
| | | | PKSPSSAKSRLQTAPVPMPDLKNVKSKIGSTENLKHQPGGGKVQIINKKLDLSNVQSKCGSKDNIKHVPGGGSVQIVY | |
| | | | KPVDLSKVTSKCGSLGNIHHKPGGGQVEVKSEKLDFKDRVQSKIGSLDNITHVPGGGNKKIETHKLTFRENAKAKTD | |
| | | | HGAEIVYKSPVVSGDTSPRHLSNVSSTGSIDMVDSPQLATLADEVSA-TauLAKQGL | |
| SA-Tau3 | Tau- | Garcia- | MAEPRQEFEVMEDHAGTYGLGDRKDQGGYTMHQDQEGDTDAGLKESPLQTPTEDGSEEPGSETSDAKSTPTAEDVT | 34 |
| | Linker-V5- | Seisdedos | APLVDEGAPGKQAAAQPHTEIPEGTTAEEAGIGDTPSLEDEAAGHVTQARMVSKSKDGTGSDDKKAKGADGKTKIA | |
| | 1POK(E23 | et al., | TPRGAAPPGQKGQANATRIPAKTPPAPKTPPSSGEPPKSGDRSGYSSPGSPGTPGSRSRTPSLPTPPTREPKKVAVVRTP | |
| | 9Y)- | 2017 | PKSPSSAKSRLQTAPVPMPDLKNGGSGGTGGMIDYTAAGFTLLQGAHLYAPEDRGICDVLVANGKIIAVASNIPSDIVP | |
| | Linker- | | NCTVVDLSGQILCPGFIDQHVHLIGGGGEAGPTTRTPEVALSRLTEAGVTSVVGLLGTDSISRHPESLLAKTRALNEEGI | |
| | MBP | | SAWMLTGAYHVPSRTITGSVEKDVAIIDRVIGVKCAISDHRSAAPDVYHLANMAAESRVGGLLGGKPGVTVFHMGD | |
| | | | SKKALQPIYDLLENCDVPISKLLPTHVNRNVPLFYQALEFARKGGTIDITSSIDEPVAPAEGIARAVQAGIPLARVTLSSD | |
| | | | GNGSQPFFDDEGNLTHIGVAGFETLLETVQVLVKDYDFSISDALRPLTSSVAGFLNLTGKGEILPGNDADLLVMTPELR | |
| | | | IEQVYARGKLMVKDGKACVKGTFETAGGSGGTGGSGGTGGSGGTGGSGGTGGKPIPNPLLGLDSTGSGKIEEGKLVI | |
| | | | WINGDKGYNGLAEVGKKFEKDTGIKVTVEHPDKLEEKFPQVAATGDGPDIIFWAHDRFGGYAQSGLLAEITPDKAFQ | |
| | | | DKLYPFTWDAVRYNGKLIAYPIAVEALSLIYNKDLLPNPPKTWEEIPALDKELKAKGKSALMFNLQEPYFTWPLIAAD | |
| | | | GGYAFKYENGKYDIKDVGVDNAGAKAGLTFLVDLIKNKHMNADTDYSIAEAAFNKGETAMTINGPWAWSNIDTSK | |
| | | | VNYGVTVLPTFKGQPSKPFVGVLSAGINAASPNKELAKEFLENYLLTDEGLEAVNKDKPLGAVALKSYEEELAKDPRI | |
| | | | AATMENAQKGEIMPNIPQMSAFWYAVRTAVINAASGRQTVDEALKDAQT | |
| SA-Tau4 | Tau- | Shen et al., | MAEPRQEFEVMEDHAGTYGLGDRKDQGGYTMHQDQEGDTDAGLKESPLQTPTEDGSEEPGSETSDAKSTPTAEDVT | 35 |
| | Linker- | 2018 | APLVDEGAPGKQAAAQPHTEIPEGTTAEEAGIGDTPSLEDEAAGHVTQARMVSKSKDGTGSDDKKAKGADGKTKIA | |
| | DHF40- | | TPRGAAPPGQKGQANATRIPAKTPPAPKTPPSSGEPPKSGDRSGYSSPGSPGTPGSRSRTPSLPTPPTREPKKVAVVRTP | |
| | Linker-V5 | | PKSPSSAKSRLQTAPVPMPDLKNGGSGGTGGMSSEKEELRERLVKICVELAKLKGDDTLKAAEAAEEAFRLVVLAAM | |
| | | | LAGIDSSEVLELAIRLIKTCVVLAAMEGYDISEACRAAAEAFTRVAMAALRAGITSSLVLKAAIELIKECVLNAAVEGY | |
| | | | DISEACRAAAEAFKRVAEAAKRAGITSLETLLRAIEEIRKRVEEAQREGNDISEACRQAAEEFRKKAEELKRRGDVGG | |
| | | | GSGGTGGGKPIPNPLLGLDST | |
| SA-Tau5 | Tau- | Shen et al., | MAEPRQEFEVMEDHAGTYGLGDRKDQGGYTMHQDQEGDTDAGLKESPLQTPTEDGSEEPGSETSDAKSTPTAEDVT | 36 |
| | Linker- | 2018 | APLVDEGAPGKQAAAQPHTEIPEGTTAEEAGIGDTPSLEDEAAGHVTQARMVSKSKDGTGSDDKKAKGADGKTKIA | |
| | DHF46- | | TPRGAAPPGQKGQANATRIPAKTPPAPKTPPSSGEPPKSGDRSGYSSPGSPGTPGSRSRTPSLPTPPTREPKKVAVVRTP | |
| | Linker-V5 | | PKSPSSAKSRLQTAPVPMPDLKNGGSGGTGGTKEERVLLMKVAILAIVAAKKGNTDEVRKALELALLIAKVSGTTEA | |
| | | | VKLALEVVARVAIEAARRGNTDAVREALEVALEIARESGTTEAVKLALEVVARVAIEAARRGNTEAVVEALLVALEI | |
| | | | AKESGTEEAVRLALEVVKRVSNEALKQGNVDAVKVALEVRKMIEELSGGGSGGTGGGKPIPNPLLGLDST | |
| SA-Tau6 | 2VYC- | Garcia- | MKVLIVESEFLHQDTWVGNAVERLADALSQQNVTVIKSTSFDDGFAILSSNEAIDCLMFSYQMEHPDEHQNVRQLIG | 37 |
| | Linker-V5- | Seisdedos | KLHERQQNVPVFLLGDREKALAAMDRDLLELVDEFAWILEDTADFIAGRAVAAMTRYRQQLLPPLFSALMKYSDIHE | |
| | linker-Tau | et al., | YSWAAPGHQGGVGFTKTPAGRFYHDYYGENLFRTDMGIERTSLGSLLDHTGAFGESEKYAARVFGADRSWSVVVGT | |
| | | 2017 | SGSNRTIMQACMTDNDVVVVDRNCHKSIEQGLMLTGAKPVYMVPSRNRYGIIGPIYPQEMQPETLQKKISESPLTKDK | |
| | | | AGQKPSYCVVTNCTYDGVCYNAKEAQDLLEKTSDRLHFDEAWYGYARFNPIYADHYAMRGEPGDHNGPTVFATHS | |
| | | | THKLLNALSQASYIHVREGRGAINFSRFNQAYMMHATTSPLYAICASNDVAVSMMDGNSGLSLTQEVIDEAVDFRQA | |
| | | | MARLYKEFTADGSWFFKPWNKEVVTDPQTGLTYLFALAPKLLTTVQDCWVMHPGESWHGFKDIPDNWSMLDPIKV | |
| | | | SILAPGMGEDGELEETGVPAALVTAWLGRHGIVPTRTTDFQIMFLFSMGVTRGKWGTLVNTLCSFKRHYDANTPLAQ | |
| | | | VMPELVEQYPDTYANMGIHDLGDTMFAWLKENNPGARLNEAYSGLPVAEVTPREAYNAIVDNNVELVSIENLPGRIA | |
| | | | ANSVIPYPPGIPMLLSGENFGDKNSPQVSYLRSLQSWDHHFPGFEHETEGTEIIDGIYHVMCVKAGGSGGTGGSGGTG | |
| | | | GGKPIPNPLLGLDSTGGSGGTGGMAEPRQEFEVMEDHAGTYGLGDRKDQGGYTMHQDQEGDTDAGLKESPLQTPTE | |
| | | | DGSEEPGSETSDAKSTPTAEDVTAPLVDEGAPGKQAAAQPHTEIPEGTTAEEAGIGDTPSLEDEAAGHVTQARMVSKS | |
| | | | KDGTGSDDKKAKGADGKTKIATPRGAAPPGQKGQANATRIPAKTPPAPKTPPSSGEPPKSGDRSGYSSPGSPGTPGSR | |
| | | | SRTPSLPTPPTREPKKVAVVRTPPKSPSSAKSRLQTAPVPMPDLKN | |
| SA-Tau10 | YPFD- | Chen et al., | MVNEVIDINEAVRAYIAQIEGLRAEIGRLDATIATLRQSLATLKSLKTLGEGKTVLVPVGSIAQVEMKVEKMDKVVVS | 38 |
| | Linker-V5- | 2020 | VGQNISAELEYEEALKYIEDEIKKLLTFRLVLEQAIAELYAKIEDLIAEAQQTSEEEKAEEEENEEKAEGGSGGTGGGKP | |
| | Linker-Tau | | IPNPLLGLDSTGGSGGTGGMAEPRQEFEVMEDHAGTYGLGDRKDQGGYTMHQDQEGDTDAGLKESPLQTPTEDGSE | |
| | | | EPGSETSDAKSTPTAEDVTAPLVDEGAPGKQAAAQPHTEIPEGTTAEEAGIGDTPSLEDEAAGHVTQARMVSKSKDGT | |
| | | | GSDDKKAKGADGKTKIATPRGAAPPGQKGQANATRIPAKTPPAPKTPPSSGEPPKSGDRSGYSSPGSPGTPGSRSRTPS | |
| | | | LPTPPTREPKKVAVVRTPPKSPSSAKSRLQTAPVPMPDLKN | |
| SA-Tau21 | Tau- | Uniprot | MAEPRQEFEVMEDHAGTYGLGDRKDQGGYTMHQDQEGDTDAGLKESPLQTPTEDGSEEPGSETSDAKSTPTAEDVT | 39 |
| | Linker- | B4DY90 | APLVDEGAPGKQAAAQPHTEIPEGTTAEEAGIGDTPSLEDEAAGHVTQARMVSKSKDGTGSDDKKAKGADGKTKIA | |
| | bTubulin- | | TPRGAAPPGQKGQANATRIPAKTPPAPKTPPSSGEPPKSGDRSGYSSPGSPGTPGSRSRTPSLPTPPTREPKKVAVVRTP | |
| | Linker-V5 | | PKSPSSAKSRLQTAPVPMPDLKNGGSGGTGGMGDVGPRWANWDPSRSGVRDHPGQQSETLPHATFLQKIKFWEVIS | |
| | | | DEHGIDPTGTYHGDSDLQLDRISVYYNEATGGKYVPRAILVDLEPGTMDSVRSGPFGQIFRPDNFVFGQSGAGNNWA | |
| | | | KGHYTEGAELVDSVLDVVWKEAESCDCLQGFQLTHSLGGGTGSGMGTLLISKIREEYPDRIMNTFSVVPSPKVSDTV | |
| | | | VEPYNATLSVHQLVENTDETYCIDNEALYDICFRTLKLTTPTYGDLNHLVSATMSGVTTCLRFPGQLNADLRKLAVN | |
| | | | MVPFPRLHFFMPGFAPLTSRGSQQYRALTVPELTQQVFDAKNMMAACDPRHGRYLTVAAVFRGRMSMKEVDEQML | |
| | | | NVQNKNSSYFVEWIPNNVKTAVCDIPPRGLKMAVTFIGNSTAIQELFKRISEQFTAMFRRKAFLHWYTGEGMDEMEF | |
| | | | TEAESNMNDLVSEYQQYQDATAEEEEDFGEEAEEEAGGSGGTGGGKPIPNPLLGLDST | |
| SA-Tau22 | Tau- | Uniprot | MAEPRQEFEVMEDHAGTYGLGDRKDQGGYTMHQDQEGDTDAGLKESPLQTPTEDGSEEPGSETSDAKSTPTAEDVT | 40 |
| | Linker- | P60709 | APLVDEGAPGKQAAAQPHTEIPEGTTAEEAGIGDTPSLEDEAAGHVTQARMVSKSKDGTGSDDKKAKGADGKTKIA | |
| | bActin- | | TPRGAAPPGQKGQANATRIPAKTPPAPKTPPSSGEPPKSGDRSGYSSPGSPGTPGSRSRTPSLPTPPTREPKKVAVVRTP | |
| | Linker-V5 | | PKSPSSAKSRLQTAPVPMPDLKNGGSGGTGGMDDDIAALVVDNGSGMCKAGFAGDDAPRAVFPSIVGRPRHQGVMV | |
| | | | GMGQKDSYVGDEAQSKRGILTLKYPIEHGIVTNWDDMEKIWHHTFYNELRVAPEEHPVLLTEAPLNPKANREKMTQI | |
| | | | MFETFNTPAMYVAIQAVLSLYASGRTTGIVMDSGDGVTHTVPIYEGYALPHAILRLDLAGRDLTDYLMKILTERGYSF | |
| | | | TTTAEREIVRDIKEKLCYVALDFEQEMATAASSSSLEKSYELPDGQVITIGNERFRCPEALFQPSFLGMESCGIHETTFN | |
| | | | SIMKCDVDIRKDLYANTVLSGGTTMYPGIADRMQKEITALAPSTMKIKIIAPPERKYSVWIGGSILASLSTFQQMWISK | |
| | | | QEYDESGPSIVHRKCFGGSGGTGGGKPIPNPLLGLDST | |
| SA-Tau25 | Tau- | Uniprot | MAEPRQEFEVMEDHAGTYGLGDRKDQGGYTMHQDQEGDTDAGLKESPLQTPTEDGSEEPGSETSDAKSTPTAEDVT | 41 |
| | Linker- | Q8N4H5 | APLVDEGAPGKQAAAQPHTEIPEGTTAEEAGIGDTPSLEDEAAGHVTQARMVSKSKDGTGSDDKKAKGADGKTKIA | |
| | Tom5- | | TPRGAAPPGQKGQANATRIPAKTPPAPKTPPSSGEPPKSGDRSGYSSPGSPGTPGSRSRTPSLPTPPTREPKKVAVVRTP | |
| | Linker-V5 | | PKSPSSAKSRLQTAPVPMPDLKNGGSGGTGGMFRIEGLAPKLDPEEMKRKMREDVISSIRNFLIYVALLRVTPFILKKL | |
| | | | DSIGGSGGTGGGKPIPNPLLGLDST | |
| SA-Tau26 | Tom20- | Uniprot | MVGRNSAIAAGVCGALFIGYCIYFGGSGGTGGMAEPRQEFEVMEDHAGTYGLGDRKDQGGYTMHQDQEGDTDAGL | 42 |
| | Linker- | Q15388 | KESPLQTPTEDGSEEPGSETSDAKSTPTAEDVTAPLVDEGAPGKQAAAQPHTEIPEGTTAEEAGIGDTPSLEDEAAGHV | |
| | Tau- | | TQARMVSKSKDGTGSDDKKAKGADGKTKIATPRGAAPPGQKGQANATRIPAKTPPAPKTPPSSGEPPKSGDRSGYSS | |
| | Linker-V5 | | PGSPGTPGSRSRTPSLPTPPTREPKKVAVVRTPPKSPSSAKSRLQTAPVPMPDLKNGGSGGTGGGKPIPNPLLGLDST | |

TABLE 5
Exemplary sequences of self-assembling TDP43 fusion protein

| | Construct | Source of SAP | | SEQ |
|---|---|---|---|---|
| Code | design | sequence | Amino Acid Sequence | ID NO |
| SNCA | TDP43- | N/A | MSEYIRVTEDENDEPIEIPSEDDGTVLLSTVTAQFPGACGLRYRNPVSQCMRGVRLVEGILHAPDAGWGNLVY | 43 |
| OE | Linker-V5 | | VVNYPKDNKRKMDETDASSAVKVKRAVQKTSDLIVLGLPWKTTEQDLKEYFSTFGEVLMVQVKKDLKTGHS | |
| | | | KGFGFVRFTEYETQVKVMSQRHMIDGRWCDCKLPNSKQSQDEPLRSRKVFVGRCTEDMTEDELREFFSQYGD | |
| | | | VMDVFIPKPFRAFAFVTFADDQIAQSLCGEDLIIKGISVHGGGSGGTGGGKPIPNPLLGLDST | |
| SA- | TDP-Linker- | Garcia- | MSEYIRVTEDENDEPIEIPSEDDGTVLLSTVTAQFPGACGLRYRNPVSQCMRGVRLVEGILHAPDAGWGNLVY | 44 |
| TDP3 | V5- | Seisdedos et al., | VVNYPKDNKRKMDETDASSAVKVKRAVQKTSDLIVLGLPWKTTEQDLKEYFSTFGEVLMVQVKKDLKTGHS | |
| | 1POK(E239 | 2017 | KGFGFVRFTEYETQVKVMSQRHMIDGRWCDCKLPNSKQSQDEPLRSRKVFVGRCTEDMTEDELREFFSQYGD | |
| | Y)-Linker- | | VMDVFIPKPFRAFAFVTFADDQIAQSLCGEDLIIKGISVHGGSGGTGGMIDYTAAGFTLLQGAHLYAPEDRGICD | |
| | MBP | | VLVANGKIIAVASNIPSDIVPNCTVVDLSGQILCPGFIDQHVHLIGGGGEAGPTTRTPEVALSRLTEAGVTSVVGL | |
| | | | LGTDSISRHPESLLAKTRALNEEGISAWMLTGAYHVPSRTITGSVEKDVAIIDRVIGVKCAISDHRSAAPDVYHL | |
| | | | ANMAAESRVGGLLGGKPGVTVFHMGDSKKALQPIYDLLENCDVPISKLLPTHVNRNVPLFYQALEFARKGGTI | |
| | | | DITSSIDEPVAPAEGIARAVQAGIPLARVTLSSDGNGSQPFFDDEGNLTHIGVAGFETLLETVQVLVKDYDFSISD | |
| | | | ALRPLTSSVAGFLNLTGKGEILPGNDADLLVMTPELRIEQVYARGKLMVKDGKACVKGTFETAGGSGGTGGS | |
| | | | GGTGGSGGTGGSGGTGGKPIPNPLLGLDSTGSGKIEEGKLVIWINGDKGYNGLAEVGKKFEKDTGIKVTVEHP | |
| | | | DKLEEKFPQVAATGDGPDIIFWAHDRFGGYAQSGLLAEITPDKAFQDKLYPFTWDAVRYNGKLIAYPIAVEALS | |
| | | | LIYNKDLLPNPPKTWEEIPALDKELKAKGKSALMFNLQEPYFTWPLIAADGGYAFKYENGKYDIKDVGVDNA | |
| | | | GAKAGLTFLVDLIKNKHMNADTDYSIAEAAFNKGETAMTINGPWAWSNIDTSKVNYGVTVLPTFKGQPSKPF | |
| | | | VGVLSAGINAASPNKELAKEFLENYLLTDEGLEAVNKDKPLGAVALKSYEEELAKDPRIAATMENAQKGEIMP | |
| | | | NIPQMSAFWYAVRTAVINAASGRQTVDEALKDAQT | |
| SA- | TDP-Linker- | Shen et al., | MSEYIRVTEDENDEPIEIPSEDDGTVLLSTVTAQFPGACGLRYRNPVSQCMRGVRLVEGILHAPDAGWGNLVY | 45 |
| TDP4 | DHF40- | 2018 | VVNYPKDNKRKMDETDASSAVKVKRAVQKTSDLIVLGLPWKTTEQDLKEYFSTFGEVLMVQVKKDLKTGHS | |
| | Linker-V5 | | KGFGFVRFTEYETQVKVMSQRHMIDGRWCDCKLPNSKQSQDEPLRSRKVFVGRCTEDMTEDELREFFSQYGD | |
| | | | VMDVFIPKPFRAFAFVTFADDQIAQSLCGEDLIIKGISVHGGSGGTGGMSSEKEELRERLVKICVELAKLKGDDT | |
| | | | LKAAEAAEEAFRLVVLAAMLAGIDSSEVLELAIRLIKTCVVLAAMEGYDISEACRAAAEAFTRVAMAALRAGI | |
| | | | TSSLVLKAAIELIKECVLNAAVEGYDISEACRAAAEAFKRVAEAAKRAGITSLETLLRAIEEIRKRVEEAQREGN | |
| | | | DISEACRQAAEEFRKKAEELKRRGDVGGGSGGTGGGKPIPNPLLGLDST | |
| SA- | TDP-Linker- | Shen et al., | MSEYIRVTEDENDEPIEIPSEDDGTVLLSTVTAQFPGACGLRYRNPVSQCMRGVRLVEGILHAPDAGWGNLVY | 46 |
| TDP5 | DHF46- | 2018 | VVNYPKDNKRKMDETDASSAVKVKRAVQKTSDLIVLGLPWKTTEQDLKEYFSTFGEVLMVQVKKDLKTGHS | |
| | Linker-V5 | | KGFGFVRFTEYETQVKVMSQRHMIDGRWCDCKLPNSKQSQDEPLRSRKVFVGRCTEDMTEDELREFFSQYGD | |
| | | | VMDVFIPKPFRAFAFVTFADDQIAQSLCGEDLIIKGISVHGGSGGTGGTKEERVLLMKVAILAIVAAKKGNTDE | |
| | | | VRKALELALLIAKVSGTTEAVKLALEVVARVAIEAARRGNTDAVREALEVALEIARESGTTEAVKLALEVVAR | |
| | | | VAIEAARRGNTEAVVEALLVALEIAKESGTEEAVRLALEVVKRVSNEALKQGNVDAVKVALEVRKMIEELSG | |
| | | | GGSGGTGGGKPIPNPLLGLDST | |
| SA- | 2VYC- | Garcia- | MKVLIVESEFLHQDTWVGNAVERLADALSQQNVTVIKSTSFDDGFAILSSNEAIDCLMFSYQMEHPDEHQNVR | 47 |
| TDP6 | Linker-V5- | Seisdedos et al., | QLIGKLHERQQNVPVFLLGDREKALAAMDRDLLELVDEFAWILEDTADFIAGRAVAAMTRYRQQLLPPLFSAL | |
| | linker-TDP | 2017 | MKYSDIHEYSWAAPGHQGGVGFTKTPAGRFYHDYYGENLFRTDMGIERTSLGSLLDHTGAFGESEKYAARVF | |
| | | | GADRSWSVVVGTSGSNRTIMQACMTDNDVVVVDRNCHKSIEQGLMLTGAKPVYMVPSRNRYGIIGPIYPQEM | |
| | | | QPETLQKKISESPLTKDKAGQKPSYCVVTNCTYDGVCYNAKEAQDLLEKTSDRLHFDEAWYGYARFNPIYAD | |
| | | | HYAMRGEPGDHNGPTVFATHSTHKLLNALSQASYIHVREGRGAINFSRFNQAYMMHATTSPLYAICASNDVA | |
| | | | VSMMDGNSGLSLTQEVIDEAVDFRQAMARLYKEFTADGSWFFKPWNKEVVTDPQTGLTYLFALAPKLLTTVQ | |
| | | | DCWVMHPGESWHGFKDIPDNWSMLDPIKVSILAPGMGEDGELEETGVPAALVTAWLGRHGIVPTRTTDFQIM | |
| | | | FLFSMGVTRGKWGTLVNTLCSFKRHYDANTPLAQVMPELVEQYPDTYANMGIHDLGDTMFAWLKENNPGAR | |
| | | | LNEAYSGLPVAEVTPREAYNAIVDNNVELVSIENLPGRIAANSVIPYPPGIPMLLSGENFGDKNSPQVSYLRSLQ | |
| | | | SWDHHFPGFEHETEGTEIIDGIYHVMCVKAGGSGGTGGSGGTGGGKPIPNPLLGLDSTGGSGGTGGMSEYIRVT | |
| | | | EDENDEPIEIPSEDDGTVLLSTVTAQFPGACGLRYRNPVSQCMRGVRLVEGILHAPDAGWGNLVYVVNYPKDN | |
| | | | KRKMDETDASSAVKVKRAVQKTSDLIVLGLPWKTTEQDLKEYFSTFGEVLMVQVKKDLKTGHSKGFGFVRFT | |
| | | | EYETQVKVMSQRHMIDGRWCDCKLPNSKQSQDEPLRSRKVFVGRCTEDMTEDELREFFSQYGDVMDVFIPKP | |
| | | | FRAFAFVTFADDQIAQSLCGEDLIIKGISVH | |
| SA- | YPFD- | Chen et al., | MVNEVIDINEAVRAYIAQIEGLRAEIGRLDATIATLRQSLATLKSLKTLGEGKTVLVPVGSIAQVEMKVEKMDK | 48 |
| TDP10 | Linker-V5- | 2020 | VVVSVGQNISAELEYEEALKYIEDEIKKLLTFRLVLEQAIAELYAKIEDLIAEAQQTSEEEKAEEEENEEKAEGGS | |
| | Linker-TDP | | GGTGGGKPIPNPLLGLDSTGGSGGTGGMSEYIRVTEDENDEPIEIPSEDDGTVLLSTVTAQFPGACGLRYRNPVS | |
| | | | QCMRGVRLVEGILHAPDAGWGNLVYVVNYPKDNKRKMDETDASSAVKVKRAVQKTSDLIVLGLPWKTTEQ | |
| | | | DLKEYFSTFGEVLMVQVKKDLKTGHSKGFGFVRFTEYETQVKVMSQRHMIDGRWCDCKLPNSKQSQDEPLRS | |
| | | | RKVFVGRCTEDMTEDELREFFSQYGDVMDVFIPKPFRAFAFVTFADDQIAQSLCGEDLIIKGISVH | |
| SA- | TDP-Linker- | Uniprot | MSEYIRVTEDENDEPIEIPSEDDGTVLLSTVTAQFPGACGLRYRNPVSQCMRGVRLVEGILHAPDAGWGNLVY | 49 |
| TDP21 | bTubulin- | B4DY90 | VVNYPKDNKRKMDETDASSAVKVKRAVQKTSDLIVLGLPWKTTEQDLKEYFSTFGEVLMVQVKKDLKTGHS | |
| | Linker-V5 | | KGFGFVRFTEYETQVKVMSQRHMIDGRWCDCKLPNSKQSQDEPLRSRKVFVGRCTEDMTEDELREFFSQYGD | |
| | | | VMDVFIPKPFRAFAFVTFADDQIAQSLCGEDLIIKGISVHGGSGGTGGMGDVGPRWANWDPSRSGVRDHPGQQ | |
| | | | SETLPHATFLQKIKFWEVISDEHGIDPTGTYHGDSDLQLDRISVYYNEATGGKYVPRAILVDLEPGTMDSVRSGP | |
| | | | FGQIFRPDNFVFGQSGAGNNWAKGHYTEGAELVDSVLDVVWKEAESCDCLQGFQLTHSLGGGTGSGMGTLLI | |
| | | | SKIREEYPDRIMNTFSVVPSPKVSDTVVEPYNATLSVHQLVENTDETYCIDNEALYDICFRTLKLTTPTYGDLNH | |
| | | | LVSATMSGVTTCLRFPGQLNADLRKLAVNMVPFPRLHFFMPGFAPLTSRGSQQYRALTVPELTQQVFDAKNM | |
| | | | MAACDPRHGRYLTVAAVFRGRMSMKEVDEQMLNVQNKNSSYFVEWIPNNVKTAVCDIPPRGLKMAVTFIGN | |
| | | | STAIQELFKRISEQFTAMFRRKAFLHWYTGEGMDEMEFTEAESNMNDLVSEYQQYQDATAEEEEDFGEEAEEE | |
| | | | AGGSGGTGGGKPIPNPLLGLDST | |
| SA- | TDP-Linker- | Uniprot P60709 | MSEYIRVTEDENDEPIEIPSEDDGTVLLSTVTAQFPGACGLRYRNPVSQCMRGVRLVEGILHAPDAGWGNLVY | 50 |
| TDP22 | bActin- | | VVNYPKDNKRKMDETDASSAVKVKRAVQKTSDLIVLGLPWKTTEQDLKEYFSTFGEVLMVQVKKDLKTGHS | |
| | Linker-V5 | | KGFGFVRFTEYETQVKVMSQRHMIDGRWCDCKLPNSKQSQDEPLRSRKVFVGRCTEDMTEDELREFFSQYGD | |
| | | | VMDVFIPKPFRAFAFVTFADDQIAQSLCGEDLIIKGISVHGGSGGTGGMDDDIAALVVDNGSGMCKAGFAGDD | |
| | | | APRAVFPSIVGRPRHQGVMVGMGQKDSYVGDEAQSKRGILTLKYPIEHGIVTNWDDMEKIWHHTFYNELRVA | |
| | | | PEEHPVLLTEAPLNPKANREKMTQIMFETFNTPAMYVAIQAVLSLYASGRTTGIVMDSGDGVTHTVPIYEGYAL | |
| | | | PHAILRLDLAGRDLTDYLMKILTERGYSFTTTAEREIVRDIKEKLCYVALDFEQEMATAASSSSLEKSYELPDGQ | |
| | | | VITIGNERFRCPEALFQPSFLGMESCGIHETTFNSIMKCDVDIRKDLYANTVLSGGTTMYPGIADRMQKEITALA | |
| | | | PSTMKIKIIAPPERKYSVWIGGSILASLSTFQQMWISKQEYDESGPSIVHRKCFGGSGGTGGGKPIPNPLLGLDST | |
| SA- | TDP-Linker- | Uniprot | MSEYIRVTEDENDEPIEIPSEDDGTVLLSTVTAQFPGACGLRYRNPVSQCMRGVRLVEGILHAPDAGWGNLVY | 51 |
| TDP25 | Tom5- | Q8N4H5 | VVNYPKDNKRKMDETDASSAVKVKRAVQKTSDLIVLGLPWKTTEQDLKEYFSTFGEVLMVQVKKDLKTGHS | |
| | Linker-V5 | | KGFGFVRFTEYETQVKVMSQRHMIDGRWCDCKLPNSKQSQDEPLRSRKVFVGRCTEDMTEDELREFFSQYGD | |
| | | | VMDVFIPKPFRAFAFVTFADDQIAQSLCGEDLIIKGISVHGGSGGTGGMFRIEGLAPKLDPEEMKRKMREDVISS | |
| | | | IRNFLIYVALLRVTPFILKKLDSIGGSGGTGGGKPIPNPLLGLDST | |
| SA- | Tom20- | Uniprot | MVGRNSAIAAGVCGALFIGYCIYFGGSGGTGGMSEYIRVTEDENDEPIEIPSEDDGTVLLSTVTAQFPGACGLRY | 52 |
| TDP26 | Linker-TDP- | Q15388 | RNPVSQCMRGVRLVEGILHAPDAGWGNLVYVVNYPKDNKRKMDETDASSAVKVKRAVQKTSDLIVLGLPW | |
| | Linker-V5 | | KTTEQDLKEYFSTFGEVLMVQVKKDLKTGHSKGFGFVRFTEYETQVKVMSQRHMIDGRWCDCKLPNSKQSQ | |
| | | | DEPLRSRKVFVGRCTEDMTEDELREFFSQYGDVMDVFIPKPFRAFAFVTFADDQIAQSLCGEDLIIKGISVHGGS | |
| | | | GGTGGGKPIPNPLLGLDST | |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. (canceled)

2. An expression vector, comprising:
one or more first promoters operably connected to a first polynucleotide comprising a first nucleic acid unit encoding a proteopathic polypeptide and a second nucleic acid unit encoding a self-assembling domain,
wherein the first polynucleotide, upon expression in a cell, encodes a self-assembling proteopathic fusion protein comprising the proteopathic polypeptide connected to the self-assembling domain and capable of forming intracellular protein aggregates in the cell.

3. The expression vector of claim 2, wherein the proteopathic polypeptide is associated with a neurodegenerative disease, optionally, the neurodegenerative disease is selected from the group consisting of: Alzheimer's disease, Parkinson's disease, amyotrophic lateral sclerosis (ALS), Huntington's disease, frontotemporal dementia (FTD), chronic traumatic encephalopathy (CTE), multiple system atrophy (MSA), and Lewy body dementia (LBD).

4. The expression vector of claim 2, wherein the proteopathic polypeptide is selected from the group consisting of: alpha synuclein polypeptide, Tau, TDP-43, an Aβ precursor protein, an Aβ peptide, a prion, serum amyloid A, amyloid β-protein, transthyretin, cystatin C, beta-2-microglobulin, apolipoprotein AI, apolipoprotein AII, gelsolin, amylin, calcitonin, atrial natriuretic factor, lysozyme, insulin, fibrinogen α-A chain, superoxide dismutase 1, huntingtin, an androgen receptor, atrophin, an ataxin, immunoglobulin light chain, neuroserpin, superoxide dismutase 1, FUS, EWS RNA binding protein 1, and a TATA box-binding protein, and/or
wherein the self-assembling domain is selected from the group consisting of: an actin protein, a tubulin protein, a cytoskeletal filament, gamma-prefoldin, viral capsid protein, cage protein, proteins driving phase separation, engineered protein with assembly-gain-of-function mutant, or a variant or a fragment thereof, optionally, the viral capsid protein is HIV-1 capsid protein or hepatitis B core antigen; the cage protein is ferritin; the protein driving phase separation is FUS or hnRNPA1; the engineered protein with assembly-gain-of-function mutation is isoaspartyl dipeptidase with E239Y mutation, ketopantoate hydroxymethyl transferase with E158L mutation, acid induced arginine decarboxylase with K491L, D494L and D497L mutation, E. coli AsnC with K126Y and D1313Y mutation.

5. The expression vector of claim 2, wherein the proteopathic polypeptide is Tau, TDP-43, alpha synuclein, huntingtin, amyloid β-protein, or a portion thereof.

6. The expression vector of claim 2, wherein the proteopathic polypeptide comprises an amino acid sequence set forth in SEQ ID Nos: 1-13 or an amino acid sequence having at least 80%, at least 85%, at least 90%, at least 95%, at least 96%, at least 97%, at least 98%, at least 99%, or 100% sequence identity to any one of SEQ ID Nos: 1-13, and/or
wherein the self-assembling domain has an amino acid sequence set forth in SEQ ID Nos: 14-22 or an amino acid sequence having at least 80%, at least 85%, at least 90%, at least 95%, at least 96%, at least 97%, at least 98%, at least 99%, or 100% sequence identity to any one of SEQ ID Nos: 14-22.

7. (canceled)

8. (canceled)

9. The expression vector of claim 2, wherein the proteopathic polypeptide is connected to the self-assembling domain via a linker, and/or wherein the self-assembling proteopathic fusion protein further comprises a molecular tag.

10. (canceled)

11. (canceled)

12. The expression vector of claim 2, wherein the first promoter is an inducible promoter, and the expression vector further comprises a second promoter operably linked to a second polynucleotide encoding a transactivator,
wherein, in the presence of the transactivator and a transactivator-binding compound, the first promoter is capable of inducing transcription of the first polynucleotide to generate the self-assembling proteopathic fusion protein.

13. The expression vector of claim 12, wherein the first promoter comprises one or more copies of a transactivator recognition sequence the transactivator is capable of binding to induce transcription, wherein the transactivator is incapable of binding the transactivator recognition sequence in the absence of the transactivator-binding compound.

14. The expression vector of claim 12, wherein the first promoter comprises a tetracycline response element (TRE), the transactivator comprises reverse tetracycline-controlled transactivator (rtTA), and the transactivator-binding compound comprises tetracycline, doxycycline or a derivative thereof.

15. The expression vector of claim 12, wherein the first promoter and/or the second promoter is cell- or tissue-specific promoter, optionally, the first promoter and/or the second promoter is a neuron-specific promoter.

16. The expression vector of claim 2, wherein the expression vector is a viral vector.

17. The expression vector of claim 16, wherein the viral vector is an adeno-associated virus (AAV), optionally the AAV vector comprises AAV1, AAV2, AAV3, AAV4, AAV5, AAV6, AAV7, AAV8, AAV9, derivatives thereof, or any combination thereof.

18. The expression vector of claim 17, wherein the AAV vector comprises an AAV9 variant engineered for systemic delivery, optionally AAV-PHP.B, AAV-PHP.eB, or AAV-PHP.S.

19. The expression vector of claim 17, wherein the AAV comprises a targeting peptide that targets the AAV to the nervous system of a subject.

20. The expression vector of claim 16, wherein the viral vector is encapsidated in a viral particle.

21. An expression system, comprising:
a first expression vector comprising a first promoter operably connected to a first polynucleotide comprising a first nucleic acid unit encoding a proteopathic polypeptide and a second nucleic acid unit encoding a self-assembling domain; and
a second expression vector comprising a second promoter operably connected to a second polynucleotide encoding a transactivator,
wherein the first promoter is an inducible promoter capable of inducing transcription of the first polynucleotide in the presence of the transactivator and a transactivator-binding compound, and
wherein the first polynucleotide, upon expression in a cell, encodes a self-assembling proteopathic fusion protein comprising the proteopathic polypeptide connected to the self-assembling domain and capable of forming intracellular protein aggregates in the cell.

22. The expression system of claim 21, the first promoter comprises one or more copies of a transactivator recognition sequence the transactivator is capable of binding to induce transcription, wherein the transactivator is incapable of binding the transactivator recognition sequence in the absence of the transactivator-binding compound.

23. The expression system of claim 21, wherein the first promoter comprises a tetracycline response element (TRE), the transactivator comprises reverse tetracycline-controlled transactivator (rtTA), and the transactivator-binding compound comprises tetracycline, doxycycline or a derivative thereof.

24. The expression system of claim 21, wherein the second promoter is a cell- or tissue-specific promoter, optionally, the second promoter is a neuron-specific promoter.

25. A composition comprising the expression vector of claim 2.

26.-37. (canceled)
